(19) United States
(12) Patent Application Publication
(10) Pub. No.: US 2021/0299169 A1
(43) Pub. Date: Sep. 30, 2021

(54) Galectin-3 Governs and Coordinates ESCRT and Autophagic Responses During Endomembrane Damage

(71) Applicant: UNM Rainforest Innovations, Albuquerque, NM (US)

(72) Inventor: VOJO P. DERETIC, PLACITAS, NM (US)

(21) Appl. No.: 17/176,726

(22) Filed: Feb. 16, 2021

Related U.S. Application Data
(60) Provisional application No. 62/978,023, filed on Feb. 18, 2020.

(51) Int. Cl. A61K 33/26; A61K 33/06; A61K 33/10; A61K 31/198; A61K 31/80; A61K 31/4412; A61K 31/16; A61K 31/4418; A61K 31/353; A61K 31/53; A61K 31/4196; A61K 31/4409; A61K 31/444; A61K 31/427; A61K 31/426; A61K 31/7004; A61P 31/04

(52) U.S. Cl. CPC A61K 33/26; A61P 31/04; A61K 33/10; A61K 31/198; A61K 31/80; A61K 31/4412; A61K 31/16; A61K 31/4418; A61K 31/353; A61K 31/53; A61K 31/4196; A61K 31/4409; A61K 31/444; A61K 31/427; A61K 31/426; A61K 31/7004; A61K 33/06

(57) ABSTRACT

The present invention is directed to the discovery that Galectin-3 (Gal3) governs and coordinates ESCRT and autophagic responses in subjects and that compositions and methods of treatment can make use of this discovery in the treatment of autophagy mediated disease states and/or conditions.

### RELATED APPLICATIONS AND GRANT SUPPORT

This application claims the benefit of priority of provisional application Ser. No. 62/978,023, of identical title, filed Feb. 18, 2020, the entire contents of said application being incorporated by reference in its entirety herein.

### FIELD OF THE INVENTION

The present invention is directed to the discovery that Galectin-3 (GAL-3) governs and coordinates ESCRT and autophagic responses in subjects and that compositions and methods of treatment can make use of this discovery in the treatment of autophagy mediated disease states and/or conditions.

### BACKGROUND AND OVERVIEW OF THE INVENTION

Endomembrane damage elicits a set of cellular responses to maintain endomembrane homeostasis including ESCRT-dependent membrane repair and autophagic removal of damaged organelles. Previous studies suggest that these systems may act separately.

The mammalian cell responds to organellar and plasma membrane damage by deploying a set of activities including those performed by the ESCRT (endosomal sorting complexes required for transport) machinery (Denais et al., 2016; Jimenez et al., 2014; Raab et al., 2016; Radulovic et al., 2018; Scheffer et al., 2014; Skowyra et al., 2018) and autophagy systems (Chauhan et al., 2016; Dupont et al., 2009; Fujita et al., 2013; Thurston et al., 2012; Wei et al., 2017; Yoshida et al., 2017). The contributions of ESCRT (Radulovic et al., 2018; Skowyra et al., 2018) and autophagic responses (Jia et al., 2018; Maejima et al., 2013; Yoshida et al., 2017) during lysosomal membrane damage have received recent attention in the context of maintaining endolysosomal system homeostasis. However, whether and how ESCRT and autophagy cooperate during endomembrane damage is not well understood. Previous studies suggest that these systems may act independently when lysosomes are damaged (Radulovic et al., 2018; Skowyra et al., 2018).

The mechanism for how ESCRT act in membrane repair, including during lysosomal damage, is believed to be membrane scission and closure (Denais et al., 2016; Jimenez et al., 2014; Raab et al., 2016; Radulovic et al., 2018; Scheffer et al., 2014; Skowyra et al., 2018), as in a range of other membrane remodeling, budding, and fission phenomena (Christ et al., 2017; Hurley, 2015). These include formation of intraluminal vesicles of late endosomal multivesicular bodies (MVB) (Katzmann et al., 2002), budding of enveloped viruses including HIV (Dussupt et al., 2009; Fisher et al., 2007; Fujii et al., 2009; Garrus et al., 2001; Martin-Serrano et al., 2001), exosome formation (Baietti et al., 2012; Nabhan et al., 2012; van Niel et al., 2011), shedding of microvesicles or ectosomes (Choudhuri et al., 2014; Matusek et al., 2014; Nabhan et al., 2012), nuclear envelope reformation after mitosis (Olmos et al., 2015; Vietri et al., 2015), and for midbody abscission during cytokinesis (Carlton and Martin-Serrano, 2007; Morita et al., 2007). ESCRT play a role in repair of plasma membrane damaged by chemical or physical means (Jimenez et al., 2014; Scheffer et al., 2014), repair or removal of cell-death inducing plasma membrane pores in pyroptosis (Ruhl et al., 2018) or membrane disruption during necroptosis (Gong et al., 2017) repair of damaged lysosomes (Radulovic et al., 2018; Skowyra et al., 2018), repair of damaged nuclear envelope (Denais et al., 2016; Raab et al., 2016), and quality control and clearance of defective nuclear pores (Webster et al., 2014). One specific ESCRT component, ALIX, is capable of bypassing ESCRT-0/-I/-II during the recruitment of ESCRT-III proteins to membrane scission sites (Christ et al., 2017; Hurley, 2015). TSG101, an ESCRT-I component, and ALIX are recruited directly to membrane damage sites at lysosomes (Radulovic et al., 2018; Skowyra et al., 2018), nuclear envelope (Denais et al., 2016; Olmos et al., 2015; Raab et al., 2016; Vietri et al., 2015), and plasma membrane (Jimenez et al., 2014), and functional studies indicate that they may act redundantly during lysosomal damage repair (Radulovic et al., 2018; Skowyra et al., 2018). Among the latest additions to the portfolio of ESCRT roles (Christ et al., 2017; Hurley, 2015) is the final step of autophagosomal membrane closure, based on theoretical considerations and some experimental support suggesting that ESCRT-assisted membrane scission occurs between the future outer and inner membrane of closing phagophores to become fully sealed autophagosomes (Knorr et al., 2015; Lee et al., 2007; Rusten and Stenmark, 2009; Takahashi et al., 2018).

Autophagy is a process contributing to both cytoplasmic quality control and metabolism (Levine and Kroemer, 2019; Mizushima et al., 2011). This pathway can be activated by metabolic inputs or stress (Garcia and Shaw, 2017; Marino et al., 2014; Noda and Ohsumi, 1998; Saxton and Sabatini, 2017; Scott et al., 2004) or can also be selectively triggered by cargo recognition (Kimura et al., 2016; Lazarou et al., 2015; Mandell et al., 2014) when guided by autophagic receptors (Birgisdottir et al., 2013; Bjorkoy et al., 2005; Kimura et al., 2016; Levine and Kroemer, 2019; Stolz et al., 2014). Autophagy machinery is often recruited to damaged organelles after they are marked for degradation by ubiquitin or galectin tags (Randow and Youle, 2014; Stolz et al., 2014). Two well studied galectin-interacting autophagy receptors are NDP52 which recognizes galectin-8 (Gal8) (Thurston et al., 2012), and TRIM16, which binds to both galectin-3 (Gal3) (Chauhan et al., 2016) and Gal8 (Kimura et al., 2017). Autophagy initiation is controlled by several modules (Mizushima et al., 2011): (i) The ULK1/2 kinase complex with FIP200, ATG13 and ATG101, acting as conduits for inhibition by active mTOR (Ganley et al., 2009; Hosokawa et al., 2009; Jung et al., 2009) and activation by AMPK (Kim et al., 2011) to induce autophagy; (ii) ATG14L-endowed Class III P13-Kinase Complex (Baskaran et al., 2014; Chang et al., 2019; Petiot et al., 2000) that includes VPS34 and Beclin 1 (He and Levine, 2010), which can also be modified by AMPK to specifically activate the ATG14L form of VPS34 (Kim et al., 2013); (iii) the ATG5-ATG12/ATG16L1 E3 ligase conjugation system (Mizushima et al., 1998a; Mizushima et al., 1998b) lipidating mammalian Atg8 (mAtg8s), including LC3B, best known as a marker for autophagosomal membranes (Kabeya et al., 2000); (iv) ATG9, and the ATG2-WIPI protein complexes, of still unknown function (Bakula et al., 2017; Velikkakath et al., 2012; Young et al., 2006). These modules become interconnected, via FIP200 that bridges the ULK1/2 complex with the mAtg8s conjugation system by binding ATG16L1 (Fujita et al., 2013; Gammoh et al., 2013; Nishimura et al., 2013), via ATG16L1 and WIPI interactions (Dooley et al., 2014), and ATG13 connecting the ULK1/2 complex with ATG14-VPS34 (Jao et al., 2013; Park et al., 2016). After initiation, autophagy terminates in a merger of autophagosomes with degradative endolysosomal compartments whereby the sequestered cargo is degraded.

Galectins, which can act as tags for autophagy receptors (Chauhan et al., 2016; Thurston et al., 2012), are a family of cytosolically synthesized lectins with affinity for β-galactoside glycoconjugates, ubiquitously present in organisms from fungi to humans (Vasta et al., 2017), and in most cases sharing distinguishing characteristics as a group (Johannes et al., 2018; Nabi et al., 2015): (i) a common structure based on carbohydrate recognition domain (CRD) as the fundamental building block conserved in metazoans (Houzelstein et al., 2004), with molecular architecture variations in the number of tandem CRDs and one notable exception of Gal3 that has a long unique N-terminal domain attached to a single CRD (Johannes et al. 2018; Nabi et al., 2015). (ii) Galectins recognize galactose-containing glycans such as N-acetyllactosamine (Galβ1-3GlcNAc or Galβ1-4GlcNAc) on glycoconjugates (glycoproteins and potentially glycolipids) integral to exofacial or lumenal membranes of several intracellular organelles including lysosomes and plasma membrane as well as present in the extracellular matrix (Johannes et al., 2018). Galectins' glycan affinities start at the disaccharide level further influenced by sugar linkages and modifications, overall carbohydrate length, and glycoconjugate moiety context within N-linked glycosylated proteins and possibly O-linked glycoproteins and sphingolipids (Di Lella et al., 2011; Johannes et al., 2018). (iii) Galectins are unconventionally secreted (Stewart et al., 2017) and physiologically and medically have been mostly recognized and studied for their extracellular functions, with effects in cell adhesion, migration, signaling, inflammation, fibrosis, infection, diabetes, cancer, liver and heart disease (Di Lella et al., 2011; Johannes et al., 2018). Nevertheless, galectins have been reported to have some intracellular activities, including effects on Ras and Wnt signaling, interactions with Bcl-2 and a role in viral budding (Wang et al., 2014) and possibly exosome formation, as well as interactions with importins and pre-spliceosome complexes (Johannes et al., 2018). Galectins have been connected to autophagy initially as factors binding to autophagy receptors (Chauhan et al., 2016; Thurston et al., 2012). More recent studies have shown that galectins act in mTOR and AMPK signaling (Jia et al., 2018). Most of the intracellular activities of galectins have hitherto been considered neither interconnected nor integrated and are usually given as a list of disparate phenomena (Johannes et al., 2018).

Intracellular galectins are some of the best markers of endolysosomal damage since they form discernible puncta on damaged lysosomes (Aits et al., 2015). However, their specific functions on lysosomes are not fully understood and galectins are considered as tags or eat me signals involved in recognition of membrane damage. Here we show that Gal3 goes beyond being a marker of lysosomal damage (Aits et al., 2015) and that it recruits, controls and integrates several subsystems including ESCRT and autophagy pathways resulting in a coordinated set of steps within the multi-tiered cellular response to endomembrane damage This response includes membrane repair, removal and replacement and, as identified here and elsewhere, is controlled by galectins.

### BRIEF DESCRIPTION OF THE INVENTION

The present invention is directed to the finding that galectin-3 (GAL-3), a member of the β-galactoside glycan-binding family of cytosolic lectins, functionally and mechanistically links and coordinates ESCRT and autophagy responses to lysosomal damage. GAL-3 and its capacity to recognize damage-exposed glycan are necessary for efficient recruitment of the key ESCRT component ALG-2-interacting protein X (ALIX, also referred to as apoptosis-linked-gene-2-interacting protein 1 AIP1 or Hp95) at early stage during lysosomal damage. Both GAL-3 and ALIX are required for restoration of lysosomal function. GAL-3 promotes interactions between ALIX and the downstream ESCRT-III effector CHMP4 early during lysosomal damage repair. At later times following lysosomal damage, GAL-3 displays its second function in autophagic responses. Thus, a novel function of GAL-3 is to participate in and coordinate the response of both the ESCRT and autophagy systems in a sequential homeostatic response to endomembrane damage.

In an embodiment, the present invention is directed to the use of Alix or an Alix agonist or inhibitor for the treatment of an autophagy mediate disease as described herein. Alix or an Alix agonist or antagonist may be used alone or combined with a Gal3 agonist or antagonist to enhance the treatment of autophagy mediated disease states and/or condition in a subject in need. Pharmaceutical compositions comprising Alix agonists or antagonists and Gal3 agonists or antagonists represent additional embodiments of the present invention.

The present invention relates to the discovery that Gal3 coordinates sequential stages of the endomembrane repair and removal response to endomembrane damage. In addition, it has also been determined that at early stages, Gal3's damage-exposed glycan recognition capacity is necessary for efficient recruitment of the key ESCRT component ALIX, required for efficient restoration of lysosomal function. Gal3 promotes interactions between ALIX and CHMP4, downstream ESCRT-III effectors engaged during lysosomal damage repair. Gal3 governs assembly of selective autophagy components engaged in removal of damaged lysosomes. These mechanisms of Gal3 and its interaction with ALIX/AIP1 may be used to provide compositions and effective therapies for the treatment of autophagy related disease states and/or conditions in subjects in need.

In an embodiment, the present invention is directed to the use of Galectin-3 (Gal3) modulators (agonists and/or inhibitors), Alix (AIP1) modulators (agonists and/or inhibitors) and/or Transferrin Receptor (TFRC, transferrin receptor protein 1) modulators (agonists and/or antagonists) to treat autophagy mediated disease states and/or conditions as otherwise described herein. It has been discovered pursuant to the present invention unexpectedly that using one or more modulators of Gal3, Alix (AIP1) modulators and/or TFRC modulators, especially at least two these different modulators in combination is particularly effective for treating autophagy disease states.

In an embodiment, the modulator is at least one agonist of Gal3, Alix (AIP1) or TFRC or mixtures these agonists. In embodiments, a mixture of a Gal3 agonist and an Alix (AIP1) agonist, a Gal3 agonist and a TFRC agonist, an Alix (AIP1) agonist and a TFRC agonist or a Gal3 agonist, an Alix (AIP1) agonist and a TFRC agonist in effective amounts is administered to a patient or subject with an autophagy mediated disease state or condition to treat, favorably influence and/or ameliorate an autophagy disease state and/or symptoms associated with an autophagy disease states. In embodiments, the Gal3 agonist is a galactose containing sugar or other Gal3 agonist as described herein. In embodiments, the Alix (AIP1) agonist is a calcium salt as otherwise described herein. In embodiments, the TFRC agonist is an iron salt as otherwise described herein.

In an embodiment, the modulator is at least one antagonist of Gal3, Alix (AIP1) or TFRC or mixtures these antagonists. In embodiments, a mixture of a Gal3 antagonist and an Alix (AIP1) antagonist, a Gal3 antagonist and a TFRC antagonist, an Alix (AIP1) antagonist and a TFRC antagonist or a Gal3 agonist, an Alix (AIP1) agonist and a TFRC agonist in effective amounts is administered to a patient or subject with an autophagy mediated disease state or condition to treat, favorably influence and/or ameliorate an autophagy disease state and/or symptoms associated with an autophagy disease states. In embodiments, the Gal3 antagonist is TD-1139 (GB0139), G3-C12, GR-MD-02, GM-CT-01, GCS-100, a modified a lactulose amine such as N-lactulose-octamethylenediamine (LDO); N,N-dilactulose-octamethylenediamine (D-LDO), and N,N-dilactulose-dodecamethylenediamine (D-LDD)), ipilimumab or a pectin, especially modified citrus pectin. In embodiments, the Alix (AIP1) antagonist is a calcium chelate as otherwise described herein. In embodiments, the TFRC antagonist is an iron chelate as otherwise described herein.

In embodiments, the modulator is a mixture of an agonist and/or an antagonist of any one or more of Gal3, Alix (AIP1) and/or TFRC.

In embodiments, the autophagy mediated disease state or condition responds favorably to upregulation of autophagy. In embodiments, the disease state or condition is a disease state or condition which is otherwise described herein. In embodiments, the disease state or condition responds favorably to inhibition of autophagy. In embodiments, the disease state or condition is severe acute respiratory syndrome coronavirus type I (SARS-CoV or SARS-CoV-1), severe acute respiratory syndrome coronavirus type II (SARS-CoV or SARS-CoV-2) or other autophagy mediated disease state or condition as described herein. In embodiments, the disease state or condition is often cancer or an autoimmune disease, among others. In embodiments, the disease state or condition is rheumatoid arthritis, antiphospholipid antibody syndrome, lupus, chronic urticarial, Sjogren's disease, as well as malaria, among others as described herein.

In embodiments, the invention is directed to pharmaceutical compositions comprising an effective amount of at least one modulator of Gal3 in combination with an effective amount of an Alix (AIP1) modulator and/or an effective amount of a TFRC modulator, in combination with a pharmaceutically acceptable carrier, additive and/or excipient. In embodiments, the pharmaceutical composition comprises an effective amount of an Alix (AIP1) modulator in combination with an effective amount of a TFRC modulator, in combination with a pharmaceutically acceptable carrier, additive and/or excipient. In embodiments, the composition comprises an effective amount of at least one Gal3 modulator, at least one Alix (AIP1) modulator and at least one TFRC modulator. In preferred embodiments, the modulators are either all agonists or all antagonists. In embodiments, the activity of the modulators in the treatment of autophagy mediated disease states is synergistic. These compositions are useful for the treatment of autophagy mediated disease states and/or conditions as otherwise described herein.

Other embodiments of the present invention may be readily gleaned from the detailed description of the invention which follows.

### BRIEF DESCRIPTION OF THE FIGURES

FIG. 1 shows that Galectin-3 and ESCRT component ALIX interact and protect lysosomes from damage. (A) Time-response of Gal3 during lysosomal damage. HeLa cells were treated with 1 mM LLOMe and endogenous Gal3 puncta quantified by high content microscopy (HCM). White masks, algorithm-defined cell boundaries (primary objects); Yellow masks, computer-identified Gal3 puncta (target objects). (B) EncyclopeDIA/scaffoldDIA analysis of Gal3 binding partners proximity-biotinylated by APEX2-Gal3 during lysosomal damage with 1 mM LLOMe for 1 h. Scatter (volcano) plot shows log 2 fold change and −log 10 P value for the proteins identified and quantified (LC/MS/MS) in 3 independent experiments (see Table S1, Tabs 1 and 2). (C) Co-IP analysis of interactions between Gal3 and ALIX/TSG101 during lysosomal damage. (D) Super-resolution microscopy analysis of ALIX and Gal3. HeLa cells transiently expressing GFP-Gal3 were treated with 1 mM LLOMe for 1 h. Scale bar, 5 μm. (E) Analysis of lysosomes purified by anti-HA immunoprecipitation (LysoIP, TMEM192-3xHA) from HEK293T cells treated with 1 mM LLOMe for 30 min. TMEM192-2xFLAG, control. (F) Status of acidified organelles in parental HeLa (WT) and Gal3-KO HeLa cells (Gal3^{KO}) assessed by LysoTracker HCM during lysosomal damage (1 mM LLOMe for 30 min followed by 30 min washout). Ctrl, control (untreated cells). Yellow masks, computer-identified LTR puncta. (G) As in F, ALIX knockdown (ALIX^{KD}). Scr, scrambled siRNA. (H) Schematic summary of the findings in FIG. 1. Data, means±SEM; HCM: n≥3 (each experiment: 500 valid primary objects/cells per well, ≥5 wells/sample). †p≥0.05 (not significant), *p<0.05, **p<0.01, ANOVA. See also FIGS. S1 and S2.

FIG. 2 shows that Galectin-3 is required for efficient recruitment of ALIX to damaged lysosomes. (A) LysoIP analysis for indicated proteins in cell lysates or lysosomes purified from parental HeLa (WT) and Gal3-knockout HeLa cells (Gal3^{KO}) subjected to the 1 mM LLOMe treatment for 30 min. (B) Quantification by HCM of overlaps between ALIX and LAMP1 in parental HeLa (WT) and Gal3-knockout HeLa cells (Gal3^{KO}) during lysosomal damage. (C) Quantification by HCM of overlaps between ALIX and LAMP1 in parental HeLa (WT) and Gal3-knockout HeLa cells (Gal3^{KO}) during Tau oligomer treatment. Cells were treated with 1 μg/mL Tau oligomer overnight and subjected to HCM analysis. (D) Schematic summary of the findings in FIG. 2. None-treated cells were used as control (Ctrl). White masks, algorithm defined cell boundaries; Yellow masks, computer-identified overlap of ALIX and LAMP1. See also FIG. S2.

FIG. 3 shows that glycosylation and specific glycosylated proteins play a role in Gal3 recognition of lysosomal damage. (A) HCM Analysis of the status of acidified organelles in wild-type CHO cells and mutant Lec3.2.8.1 by LysoTracker during lysosomal damage. As in FIG. 1F. (B) Quantification by HCM of overlaps between ALIX and LAMP1 in parental HeLa (WT) and Gal3-knockout HeLa cells (Gal3^{KO}). As in FIG. 2B. (C) LysoIP analysis for indicated proteins in cell lysates or lysosomes purified from parental HeLa cells subjected to 1 mM LLOMe or starvation (EBSS medium) treatment for 30 min. (D) Co-immunoprecipitation analysis of changes in interactions between Gal3 and TFRC during the process of lysosomal damage. (E) Co-immunoprecipitation analysis of changes in interactions between Gal3/Gal3^{R186S} and TFRC during the process of lysosomal damage. (F) Quantification by HCM of overlaps between Gal3 and LAMP1 in parental HeLa (WT) and TFRC-knockdown HeLa cells (TFRC^{KD}) during LLOMe treatment. (G) Quantification by HCM of overlaps between ALIX and LAMP1 in parental HeLa (WT) and TFRC-knockdown HeLa cells (TFRC^{KD}) during LLOMe treatment. (H) Quantification by HCM of overlaps between ALIX and TFRC in parental HeLa (WT) and Gal3-knockout HeLa cells (Gal3^{KO}) during LLOMe treatment. (I) LysoIP analysis for indicated proteins in cell lysates or lysosomes purified from parental HeLa (WT) and Gal3-knockout HeLa cells (Gal3^{KO}) with or without TFRC-knockdown (TFRC^{KD}). None-treated cells were used as control (Ctrl). White masks, algorithm defined cell boundaries; Yellow masks, computer-identified overlap of two proteins. See also FIG. S3.

FIG. 4 shows that optimal ALIX recruitment to damaged lysosomes requires two signals. (A) Cells were treated with 15 μM BAPTA-AM for 1 h, subjected to 1 mM LLOMe treatment for the indicated time, followed by HCM analysis of overlaps between ALIX and LAMP1. As in FIG. 2B. (B) The constructed HeLa Flp-In-Gal3^{TetON} cells stably expressing mCherry-Gal3 induced by tetracycline were subject to 15 μM BAPTA-AM treatment for 1 h and then treated with LLOMe. (C) Co-immunoprecipitation analysis of changes in interactions between Gal3 and ALIX under BAPTA-AM treatment during lysosomal damage. (D) Quantification by HCM of overlaps between ALIX and LAMP1 in parental HeLa (WT) and Gal3-knockout HeLa cells (Gal3^{KO}) treated with BAPTA-AM during lysosomal damage. (E) Quantification by HCM of overlaps between ALIX and LAMP1 in parental HeLa (WT) and Gal3-knockout HeLa cells (Gal3^{KO}) treated with BAPTA-AM during lysosomal damage. (F) LysoIP analysis for indicated proteins in cell lysates or lysosomes purified from parental HeLa (WT) and Gal3-knockout HeLa cells (Gal3^{KO}) treated with 15 μM BAPTA-AM for 1 h subjected to the 1 mM LLOMe treatment for 10 min. (G) LysoIP analysis for indicated proteins in cell lysates or lysosomes purified from parental HeLa (WT) and Gal3-knockout HeLa cells (Gal3^{KO}) treated with 15 μM BAPTA-AM for 1 h subjected to LLOMe treatment. (H) Schematic summary of the findings in FIG. 4.

FIG. 5 shows that galectin-3 promotes response of core ESCRT-III effectors during lysosomal damage. (A) (i) LysoIP analysis for indicated proteins in cell lysates or lysosomes purified from parental HeLa (WT) and Gal3-knockout HeLa cells (Gal3^{KO}) after 1 mM LLOMe treatment for 30 min. (ii-iii) Quantification of LysoIP analysis for CHMP4A and CHMP4B. (B) Co-immunoprecipitation analysis of changes in interactions between Gal3 and CHMP4A during the process of lysosomal damage. (C) Co-immunoprecipitation analysis of changes in interactions between Gal3 and CHMP4B during the process of lysosomal damage. (D) Co-immunoprecipitation analysis of the effect of Gal3 on the interaction between ALIX and CHMP4B during lysosomal damage. (E) Co-immunoprecipitation analysis of the interaction between ALIX and CHMP4B in parental HeLa (WT) and Gal3-knockout HeLa cells (Gal3^{KO}) during lysosomal damage. (F) Co-immunoprecipitation analysis of the effect of Gal3 and its mutant Gal3^{R186S} on the interaction between ALIX and CHMP4B. See also FIG. S5.

FIG. 6 shows that galectin-3 is important for autophagy response to lysosomal damage. (A) HeLa cells were treated with 1 mM LLOMe for 2 h and subjected to HCM analysis of ATG13 puncta. (B) HeLa cells were treated with 1 mM LLOMe for 2 h and subjected to HCM analysis of ATG16L1 puncta. (C) HeLa cells were treated with 1 mM LLOMe for 2 h and subjected to HCM analysis of LC3. (D) Gal3-knockout HeLa cells (Gal3^{KO}) transfected with GFP-tagged WT Gal3 or Gal3^{R186S} were treated with 1 mM LLOMe for 2 h and subjected to HCM analysis of ATG13 puncta. (E) Gal3-knockout HeLa cells (Gal3^{KO}) transfected with GFP-tagged WT Gal3 or Gal3^{R186S} were treated with 1 mM LLOMe for 2 h and subjected to HCM analysis of ATG16L1 puncta. (F) Gal3-knockout HeLa cells (Gal3^{KO}) transfected with GFP-tagged WT Gal3 or -Gal3^{R186S} were treated with 1 mM LLOMe for 2 h and the puncta of LC3 was quantified using HCM. None-treated cells were used as control (Ctrl). White masks, algorithm defined cell boundaries; Red masks, computer-identified target protein puncta. See also FIG. S5.

FIG. 7 shows that galectin-3 serves as a switch between ESCRT and autophagy responses to lysosomal damage. (A) Co-immunoprecipitation analysis of changes in interactions between Gal3 and ESCRT components during the process of lysosomal damage. (B) The constructed HeLa Flp-In-Gal3^{TetON} cells stably expressing Gal3 induced by tetracycline (Tet) were subject to ALIX knockdown and then treated with 1 mM LLOMe for 1 h. (C) Co-immunoprecipitation analysis of the effect of ALIX on interaction between Gal3 and TRIM16 during lysosomal damage. (D) Co-immunoprecipitation analysis of the effect of TRIM16 on the interaction between ALIX and Gal3 during lysosomal damage. (E) Co-immunoprecipitation analysis of the effect of TRIM16 on interaction between Gal3 and ALIX during lysosomal damage. (F) TFEB nuclear translocation in parental HeLa (WT) and Gal3-knockout HeLa cells (Gal3^{KO}) during lysosomal damage. Blue: nuclei, Hoechst 33342; Red: anti-TFEB antibody, Alexa-568. White masks, computer algorithm-defined cell boundaries; Pink masks, computer-identified nuclear TFEB based on the average intensity of Alexa-568 fluorescence. (G) Schematic summary of the findings in FIG. 7. See also FIG. S6.

FIG. 8 shows that lysosomal damage promotes the interaction between Gal3 and ESCRT component ALIX (A) LC-MS/MS proteomic analysis of APEX2-Gal3. (B) Co-immunoprecipitation analysis of changes in interactions between Gal3 and ALIX during lysosomal damage. (C) Quantification by high content (HC) of overlaps between Gal3 and ALIX. (D) dSTORM analysis of ALIX localization relative to Gal3.

FIG. 9 shows that Gal3 recruits ALIX to damaged lysosomes dependent of glycosylation. (A) LysoIP analysis in parental HeLa (WT) and Gal3-knockout HeLa cells (Gal3KO) cells. (B) Analysis of the status of acidified organelles in wild-type CHO cells and mutant Lec3.2.8.1. (C) Quantification of overlaps between ALIX and LAMP1 by complementation of Gal3 and its mutant.

FIG. 10 shows optimal ALIX recruitment to damaged lysosomes requires two signals (A) Quantification of overlaps between ALIX and LAMP1 in HeLa treated with BAPTA-AM (Ca2+ chelator) during lysosomal damage. (B) Gal3 response under BAPTA-AM treatment during lysosomal damage. (C) Analysis of changes in interactions between Gal3 and ALIX under BAPTA-AM treatment during lysosomal damage. (D) Quantification of overlaps between ALIX and LAMP1 in parental HeLa (WT) and Gal3KO cells treated with BAPTA-AM during lysosomal damage.

FIG. 11 shows that Gal3 promotes sequential aspects of ESCRT response to lysosomal damage (A) LysoIP analysis for indicated proteins in cell lysates or lysosomes purified from parental HeLa (WT) and Gal3KO cells. (B-C) Co-immunoprecipitation analysis of the effect of Gal3 on the interaction between ALIX and CHMP4B during lysosomal damage.

FIG. 12 shows that Gal3 is the switch between ESCRT repair and removal by autophagy (A) ATG13 response in parental HeLa (WT) and Gal3KO cells during lysosomal damage. (B) Analysis of changes in interactions between Gal3 and ESCRT components or TRIM16 during lysosomal damage. (C) ALIX response in parental HeLa (WT) and TRIM16KO cells during lysosomal damage. (D) ATG13 response in ALIX knockdown (ALIXKD) cells during lysosomal damage.

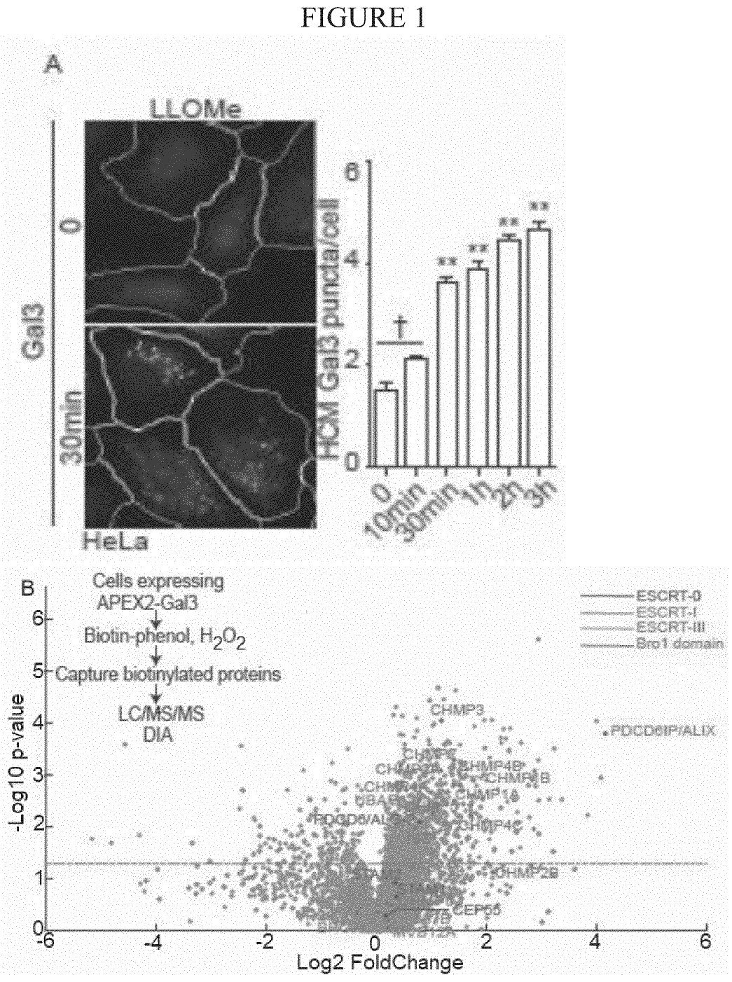

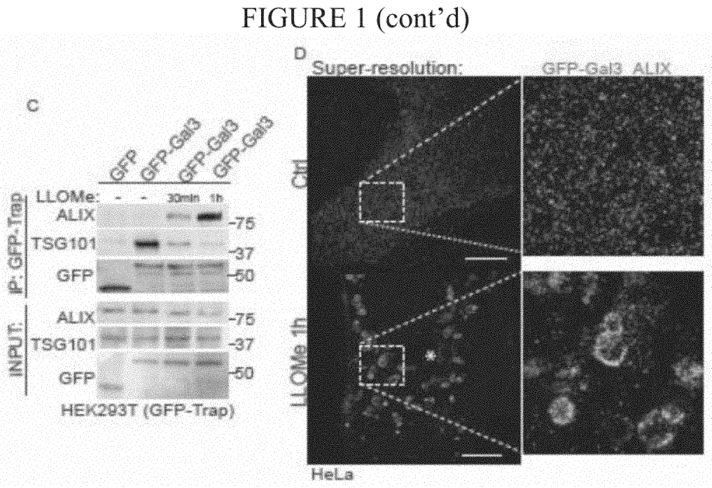

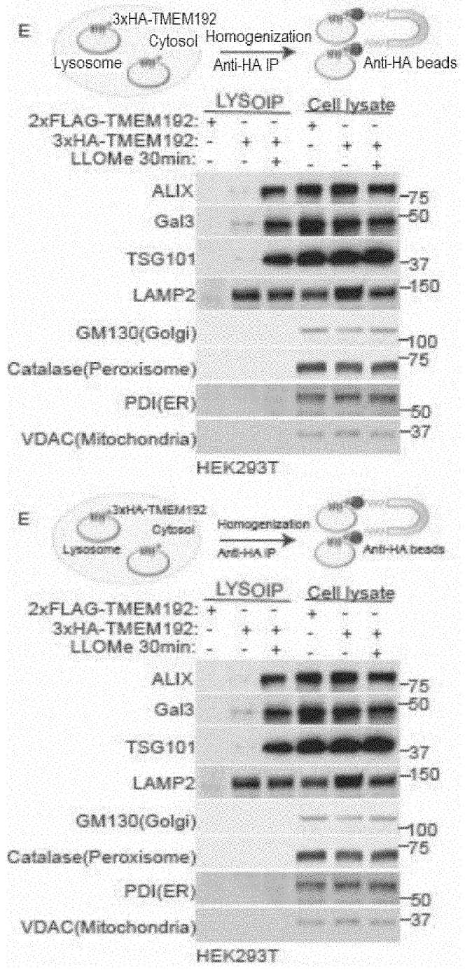

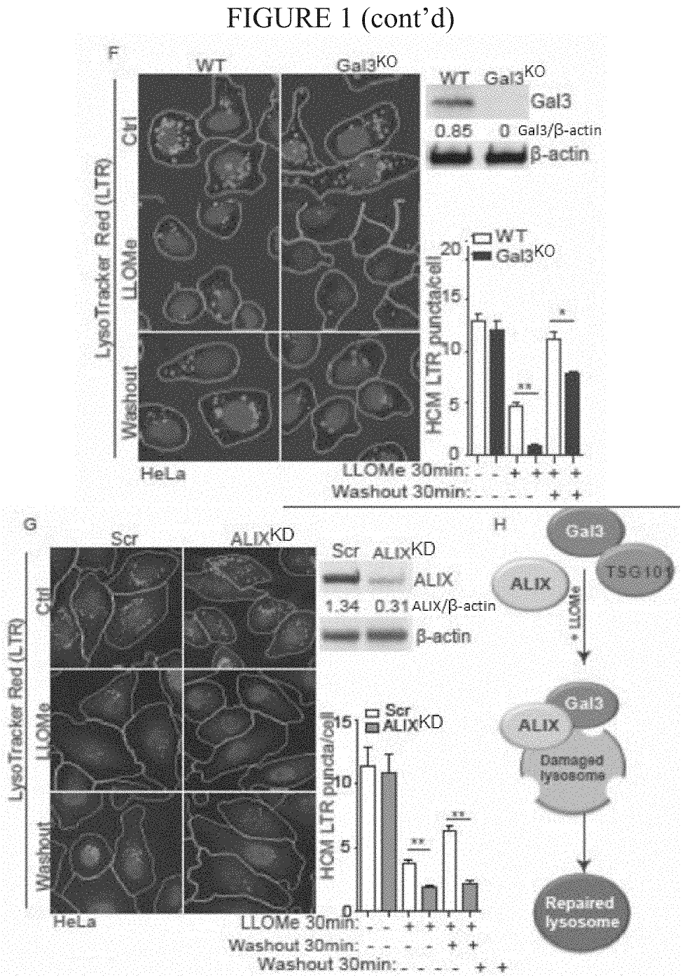

FIG. S1, related to FIG. 1 shows that Gal3 and ALIX interact and counteract damage to lysosomes. (A) Examples of HCM images used for time-response HCM quantification of Gal3 during lysosomal damage in FIG. 1A. (B) Co-immunoprecipitation analysis and quantification of changes in interactions between Gal3 and ALIX during lysosomal damage. HEK293T cells transiently expressing FLAG-Gal3 were treated with 1 mM LLOMe for the indicated time. Cell lysates were immunoprecipitated (IP) with anti-FLAG antibody, immunoblotted for endogenous ALIX, intensities quantified and normalized. (C) Co-immunoprecipitation analysis of changes in interactions between Gal3 and ALIX or TSG101 during lysosomal damage. HEK293T cells transiently expressing GFP-Gal3 were treated with 1 mM LLOMe for the indicated time. Cell lysates were immunoprecipitated (IP) with GFP-Trap beads and immunoblotted for endogenous ALIX or TSG101. (D) Co-immunoprecipitation analysis of changes in interactions between Gal3 and TSG101 during lysosomal damage. HEK293T cells transiently expressing GFP-Gal3 and FLAG-TSG101 were treated with 1 mM LLOMe for the indicated time. Cell lysates were immunoprecipitated (IP) with anti-FLAG antibody and immunoblotted for GFP-Gal3. (E) Co-immunoprecipitation analysis of interactions between Gal3 variants and TSG101 during lysosomal damage HEK293T cells transiently expressing FLAG tagged variants of Gal3 and GFP-TSG101 were treated with 1 mM LLOMe for 1 h. Cell lysates were immunoprecipitated (IP) with anti-GFP antibody and immunoblotted for FLAG tagged variants of Gal3. (F) Unmerged (separate channels) images of super-resolution analysis in FIG. 1D. (G) Quantification by HCM of overlaps between Gal3 and ALIX HeLa cells were treated with 1 mM LLOMe for the indicated time and subjected to HCM analysis of overlaps between Gal3 and ALIX. White masks, computer algorithm defined cell boundaries (primary objects); Yellow masks, computer-identified overlap of ALIX and Gal3. (H, I) HCM analysis for the total area of LTR puncta as shown in FIG. 1F and FIG. 1G. (J) Magic Red (MR) analysis of the status of acidified organelles in parental HeLa (WT) and Gal3-knockout HeLa cells (Gal3^{KO}) during lysosomal damage. Cells were treated with 1 mM LLOMe for 30 min followed by 30 min washout, and MR⁺ puncta quantified by HCM. Untreated cells were used as control (Ctrl). White masks, algorithm defined cell boundaries; Yellow masks, computer-identified MR⁺ puncta. (K) Analysis of the status of acidified organelles in ALIX knockdown (ALIX^{KD}) cells by MR during lysosomal damage. Cells transfected with scrambled siRNA as control (Scr) or ALIX siRNA were treated with 1 mM LLOMe for 30 min followed by 30 min washout, and then quantified for MR puncta using HCM. White masks, algorithm-defined cell boundaries; Yellow masks, computer-identified MR⁺ puncta.

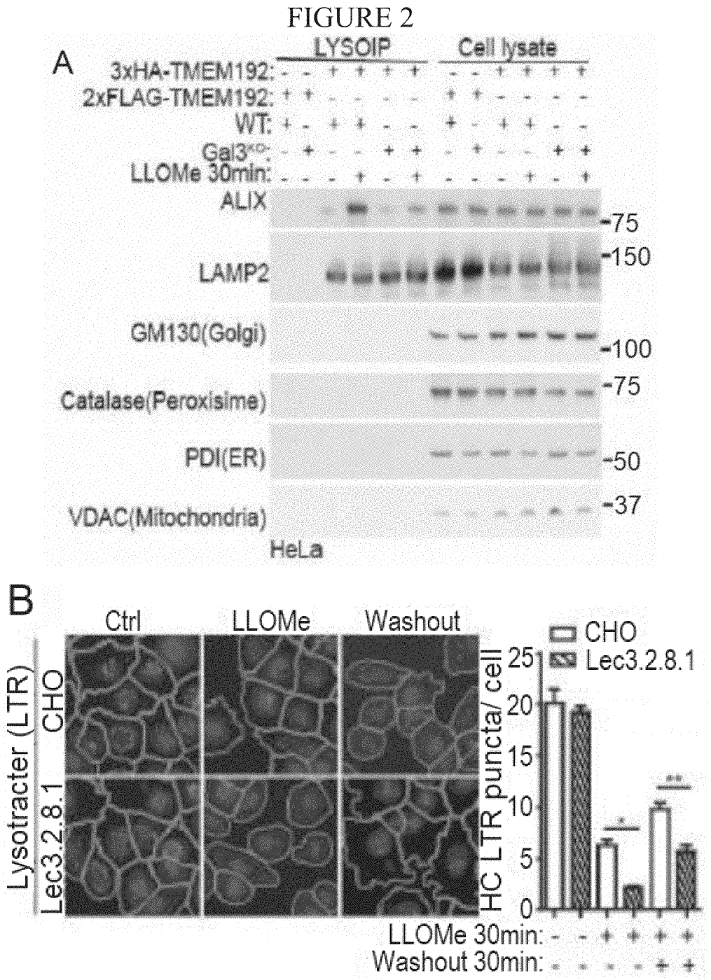

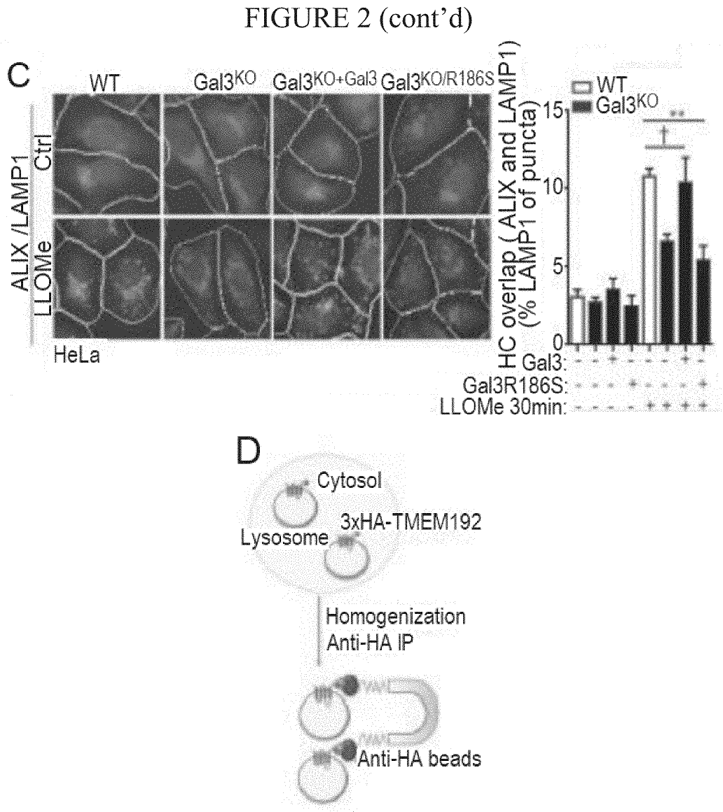

FIG. S2, related to FIGS. 1 and 2 shows that Gal3 protects against lysosomal damage and recruits ALIX to damaged lysosomes. (A, B) Analysis of the status of acidified organelles by LysoTracker Green (LTG) in Gal3 or Gal8 FLIP-IN stable cell lines during lysosomal damage. HeLa FLIP-IN cells stably expressing Gal3 or Gal8 induced by tetracycline (Tet), were treated with 1 mM LLOMe for 30 min followed by 30 min washout, and LTG⁺ puncta quantified by HCM. Ctrl, control untreated (no LLOMe) cells. White masks, algorithm-defined cell boundaries (primary objects); Green masks, computer-identified LTG⁺ puncta (target objects). (C) Analysis of the status of acidified organelles in parental HeLa (WT) and Gal8-knockout HeLa cells (Gal8^{KO}) by LysoTracker (LTR) staining during lysosomal damage. Untreated cells were used as control (Ctrl). White masks, algorithm defined cell boundaries; Yellow masks, computer-identified LTR puncta. (D) Analysis of the status of acidified organelles in parental Huh7 (WT) and Gal9-knockout Huh7 cells (Gal9^{KO}) by LTR staining and HCM during lysosomal damage. Ctrl, control untreated (no LLOMe) cells. White masks, algorithm-defined cell boundaries; Yellow masks, computer-identified LTR⁺ puncta. (E) Analysis of the status of acidified organelles in parental HeLa (WT) and Gal3-knockout HeLa cells (Gal3^{KO}) by LTR during glycyl-L-phenylalanine 2-naphthylamide (GPN) treatment. Cells were treated with 200 μM GPN for 30 min followed by 30 min washout, and LTR⁺ puncta quantified by HCM. White masks, algorithm defined cell boundaries; Yellow masks, computer-identified LysoTracker Red puncta (target objects). (F) Analysis of the status of acidified organelles in parental HeLa (WT) and Gal3-knockout HeLa cells (Gal3^{KO}) by LTR during silica treatment. Cells were treated with 400 μg/mL silica for 30 min, and LTR⁺ puncta quantified by HCM. White masks, algorithm-defined cell boundaries; Yellow masks, computer-identified LysoTracker Red puncta. (G) Schematic summary of the findings in FIG. S2A-F. (H) Quantification by HCM of overlaps between ALIX and LAMP1 in parental HeLa (WT), Gal3-knockout HeLa cells (Gal3^{KO}) and Gal8-knockout HeLa cells (Gal8^{KO}) during lysosomal damage. Cells were treated with 1 mM LLOMe for 30 min, and HCM analysis of overlaps between ALIX and LAMP1 carried out. White masks, algorithm-defined cell boundaries; Yellow masks, computer-identified overlap of ALIX and LAMP1. Data, means±SEM; HCM: n≥3 (each experiment: 500 valid primary objects/cells per well, ≥5 wells/sample). †p≥0.05 (not significant), *p<0.05, **p<0.01, ANOVA.

FIG. S3, related to FIGURE shows that glycosylation is important for Gal3 recognition of lysosomal damage. (A, B) Response of Gal3 or ALIX in wild-type CHO cells and mutant Lec3.2.8.1 during lysosomal damage. Cells transfected with GFP-Gal3 (A) and FLAG-ALIX (B) were treated with 1 mM LLOMe for 30 min, and responses of Gal3 (A) and ALIX (B) by their puncta formation were quantified by HCM. Ctrl, untreated control cells. White masks, algorithm-defined cell boundaries; Green or red masks, computer-identified Gal3 or ALIX puncta, respectively. (C) Response of Gal3 and its mutant Gal3^{R186S} during lysosomal damage. HeLa cells transfected with GFP-tagged WT Gal3 or Gal3^{R186S} were treated with 1 mM LLOMe for 30 min. White masks, algorithm-defined cell boundaries; Green masks, computer-identified GFP puncta. (D) Analysis of the status of acidified organelles by Gal3 and its mutant Gal3^{R186S}. Gal3-knockout HeLa cells (Gal3^{KO}) transfected with Gal3 or Gal3^{R186S} were treated with 1 mM LLOMe for 30 min. LTR (LysoTracker Red) puncta were quantified by HCM. White masks, algorithm-defined cell boundaries; Yellow masks, computer-identified LTR⁺ puncta. (E) Proteomic analysis of Gal3 binding partners by proximity-biotinylation using APEX2-Gal3 during lysosomal damage with 200 μM glycyl-L-phenylalanine 2-naphthylamide (GPN) for 1 h or with 1 mM LLOMe for 1 h. Scatter (volcano) plot shows fold change (log 2 FoldChange) and −log 10 p-value for the proteins quantified (spectral counts; LC/MS/MS) in three independently replicated experiments with lysosomal damage (see Table S1, Tab 4). (F) Summary of APEX2-Gal3 LC/MS/MS proteomic DIA or DDA analysis (see STAR methods, and Table S1, Tabs 1, 2 and 4). (G, H) Quantification by HCM of overlaps between TFRC and LAMP2 or Gal3. HeLa cells were treated with 1 mM LLOMe for the indicated time, and HCM analysis carried out for overlaps between TFRC and LAMP2 or Gal3. White masks, algorithm-defined cell boundaries; Yellow masks, computer-identified overlap of TFRC with LAMP2 or Gal3 puncta. (I) Analysis of the status of acidified organelles in parental HeLa (WT) and TFRC-knockdown HeLa cells (TFRC^{KD}) by LTR staining during lysosomal damage. Cells were treated with 1 mM LLOMe for 1 h and LTR⁺ puncta quantified by HCM. White masks, algorithm defined cell boundaries; Yellow masks, computer-identified LTR puncta. Data, means±SEM; HCM: n≥3 (each experiment: 500 valid primary objects/cells per well, ≥5 wells/sample) †p≥0.05 (not significant), *p<0.05, **p<0.01, ANOVA.

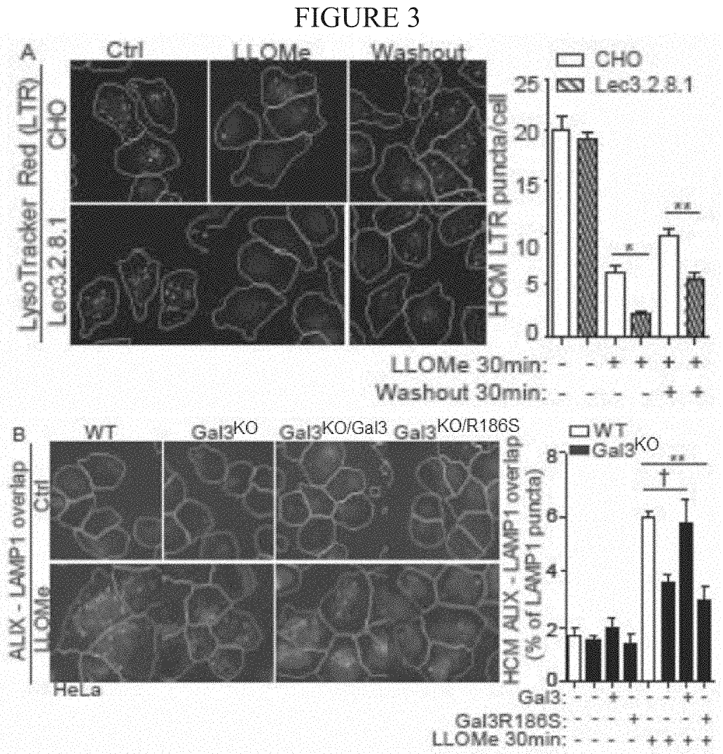

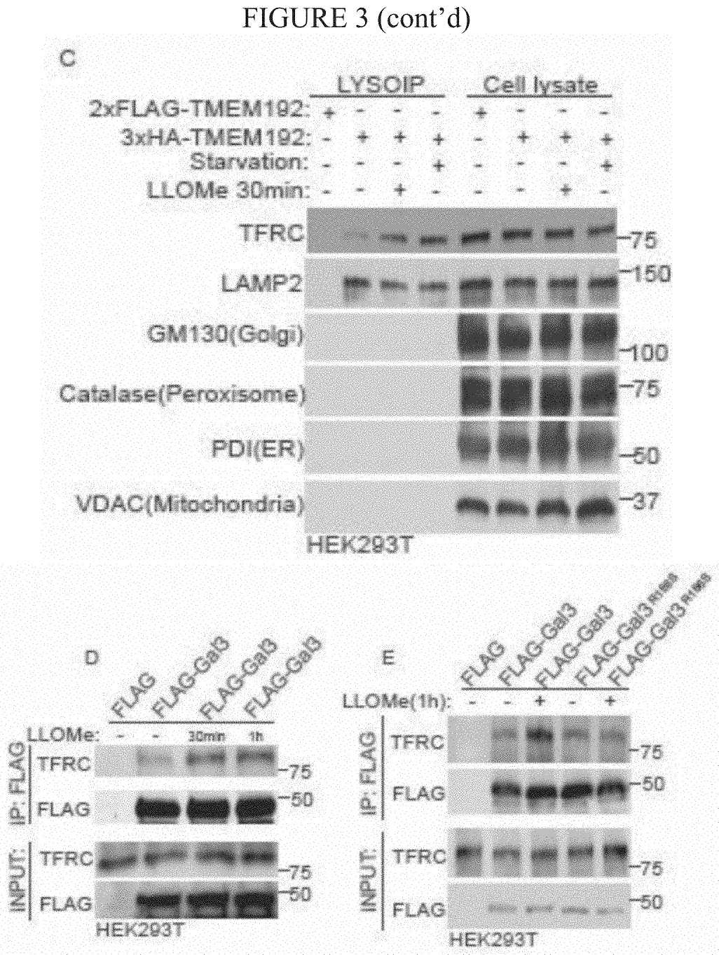

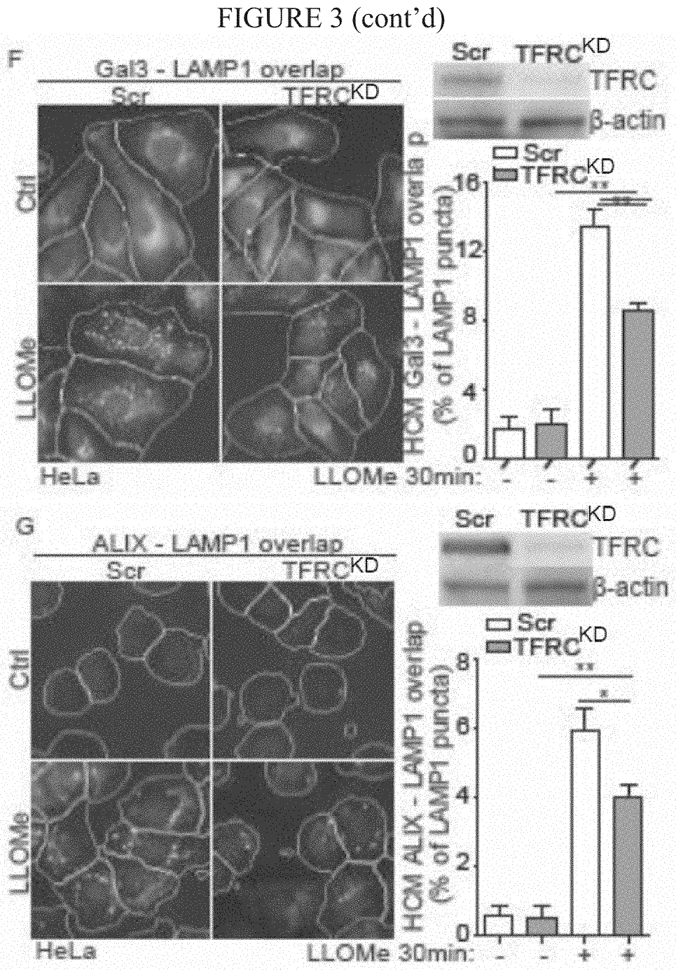

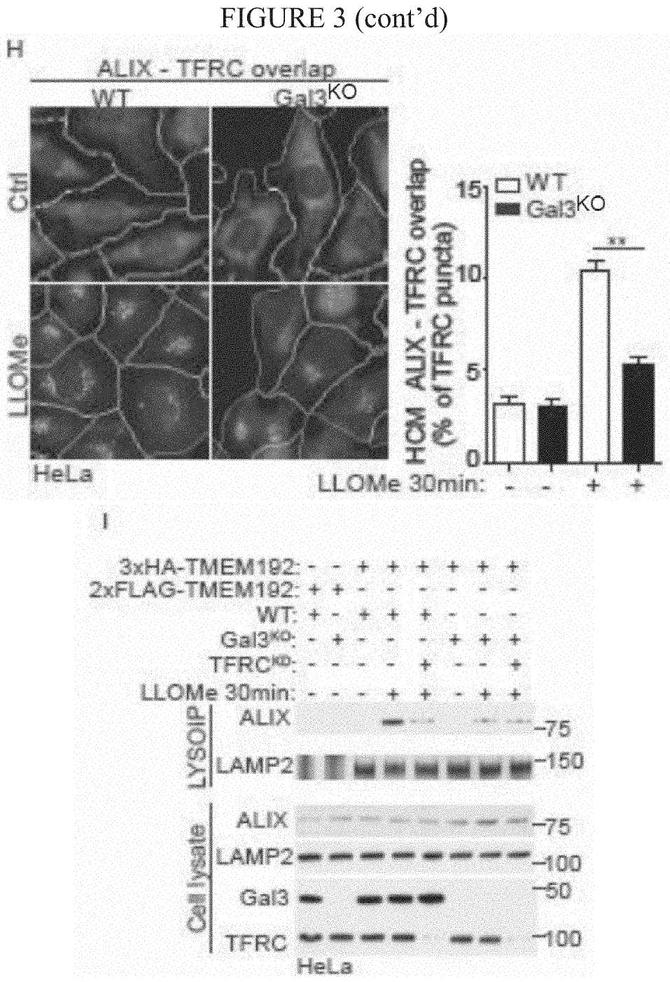

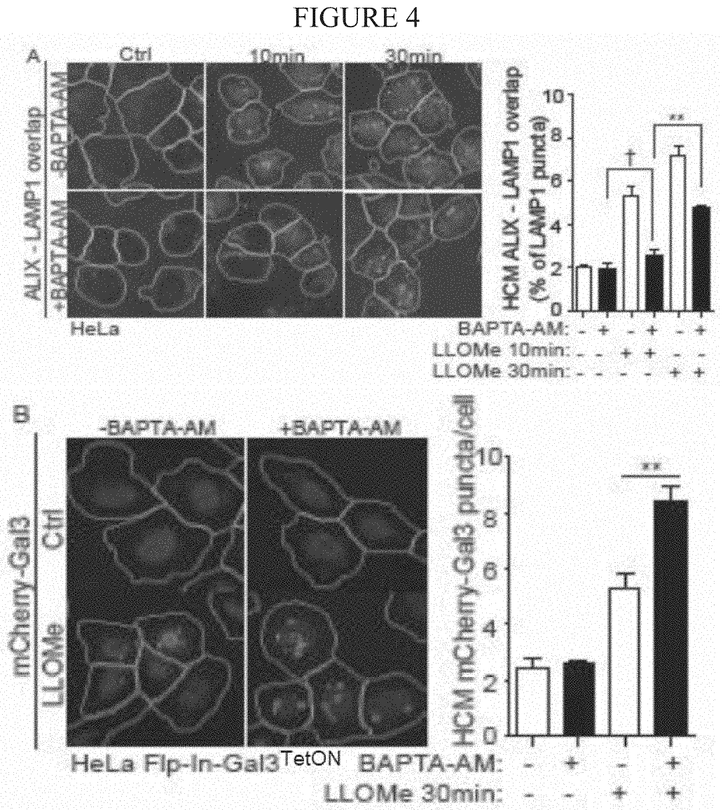

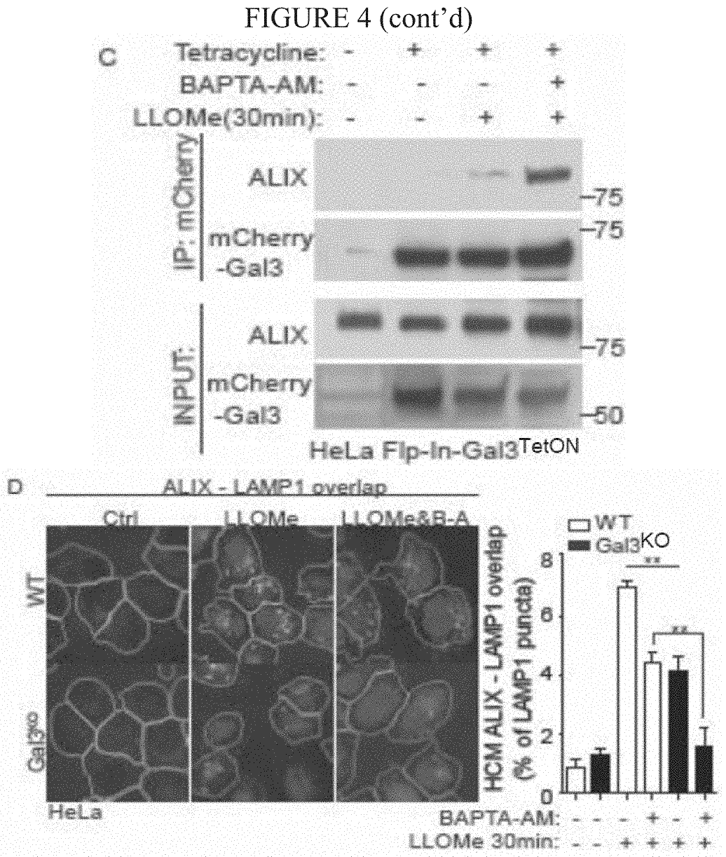

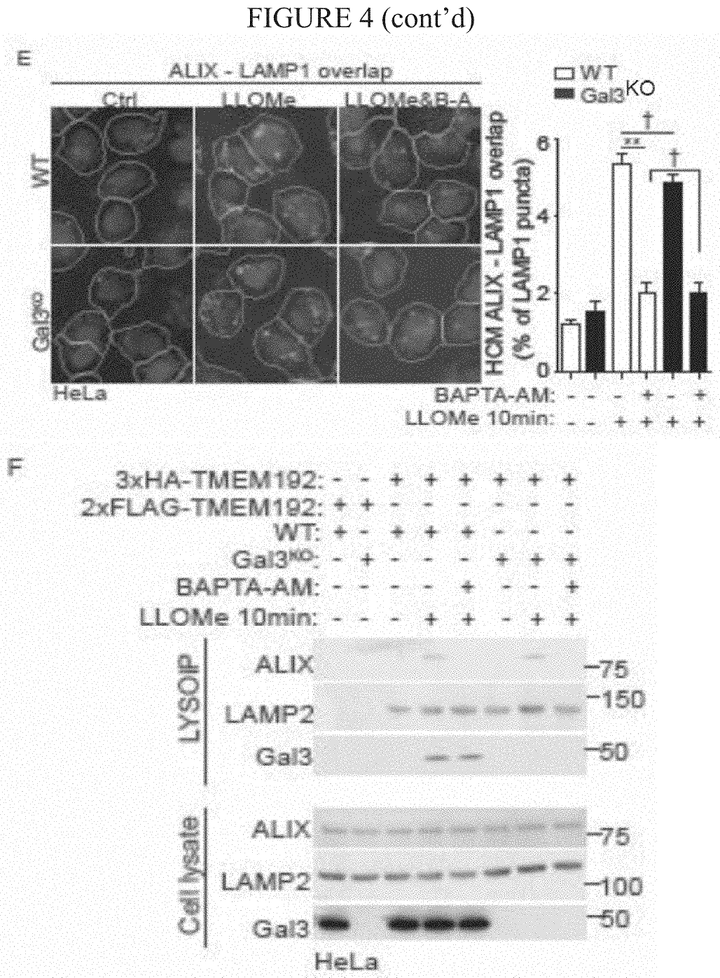

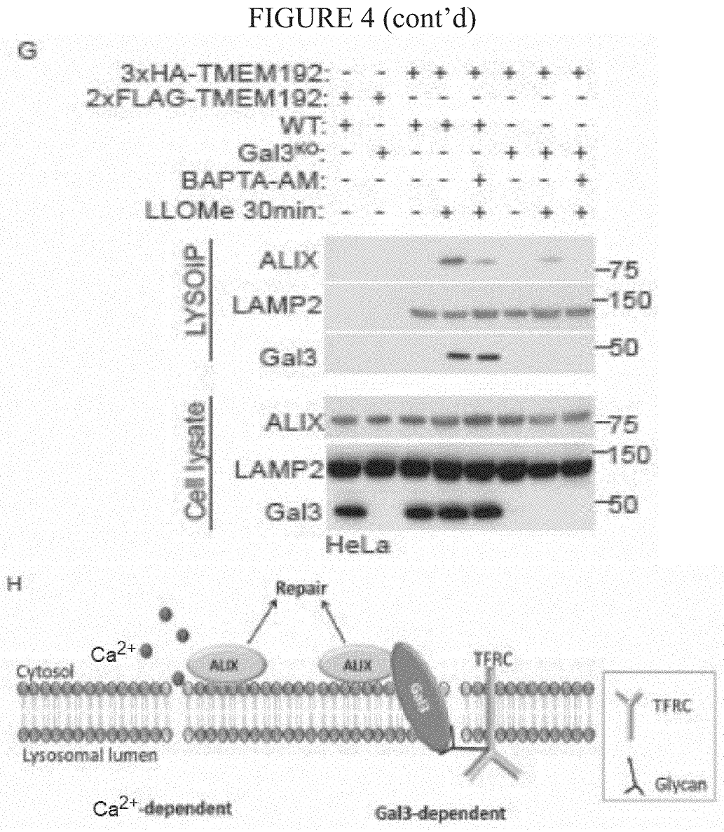

FIG. S4, related to FIG. 3 shows that TFRC plays a role in Gal3 recognition of lysosomal damage. (A) Analysis of overlaps between TFRC and LAMP2 in parental HeLa (WT) and Gal3-knockout HeLa cells (Gal3^{KO}). Cells were treated with 1 mM LLOMe for 1 h and overlaps between endogenous TFRC and LAMP2 quantified by HCM. Untreated cells (no LLOMe) were used as control (Ctrl). White masks, algorithm-defined cell boundaries (primary objects); Yellow masks, computer-identified overlap of TFRC and LAMP2 puncta. (B) Quantification by HCM of overlaps between ALIX and TFRC affected by Gal3 and its mutant Gal3^{R186S}. Gal3-knockout HeLa cells (Gal3^{KO}) transfected with GFP-tagged Gal3 or Gal3^{R186S} were treated with 1 mM LLOMe for 30 min. HCM analysis of overlaps between ALIX and TFRC. White masks, algorithm-defined cell boundaries; Yellow masks, computer-identified overlap of ALIX and TFRC. (C) Quantification by HCM of overlaps between ALIX and LAMP1 in parental HeLa (WT), Gal3-knockout HeLa cells (Gal3^{KO}) and TFRC-knockdown HeLa cells (TFRC^{KD}) during LLOMe treatment. Cells were treated with 1 mM LLOMe for 30 min, and HCM analysis of overlaps between ALIX and LAMP1 carried out. Scr, cells transfected with scrambled siRNA as control. White masks, algorithm-defined cell boundaries; Yellow masks, computer-identified overlap of ALIX and LAMP1. (D) Co-immunoprecipitation analysis of changes in interactions between Gal3 and ALIX in TFRC-knockdown HeLa cells (TFRC^{KD}) during LLOMe treatment. HeLa Flp-In-Gal3^{TetON} cells stably expressing Gal3 (induced when necessary by tetracycline; Tet) were subject to TFRC knockdown and then treated with 1 mM LLOMe for 1 h. Cell lysates were immunoprecipitated (IP) with anti-mCherry antibody and immunoblotted for endogenous ALIX. (E) Schematic summary of the findings in FIGS. 3, S3 and S4.

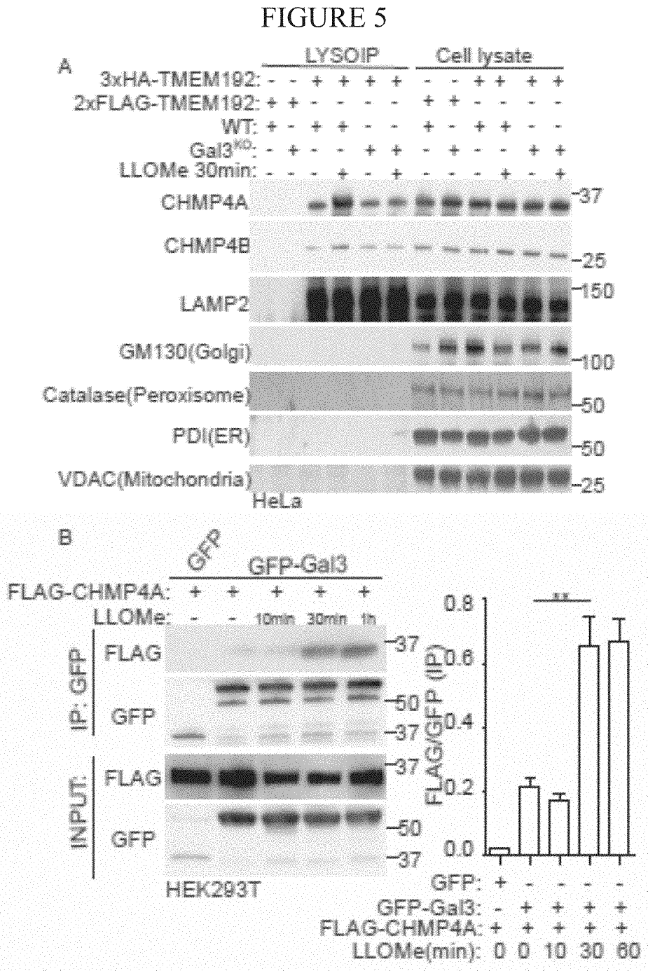

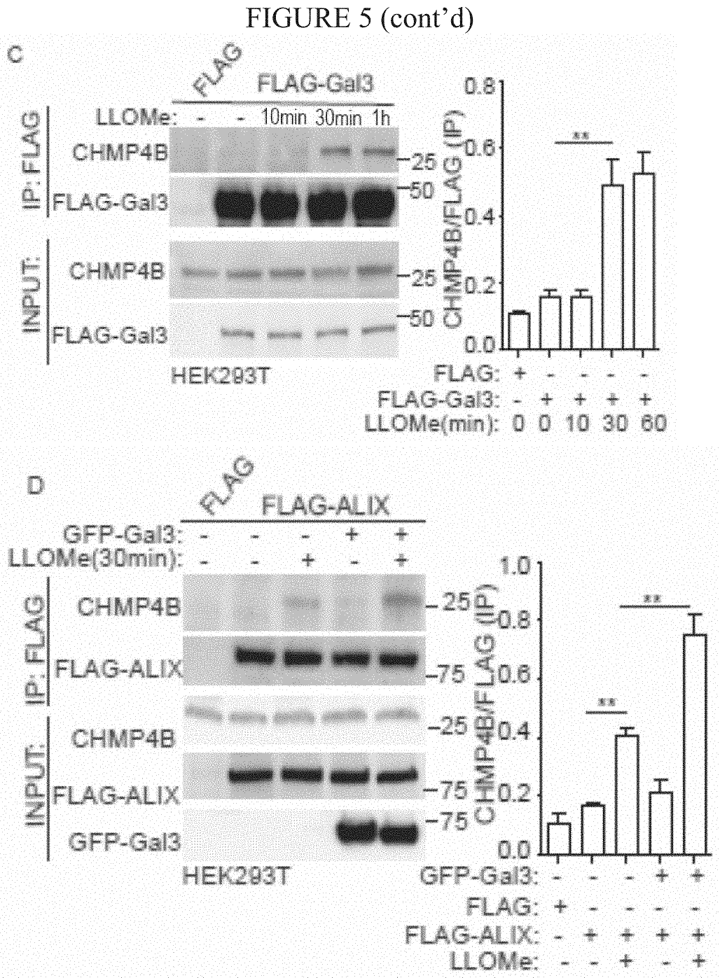

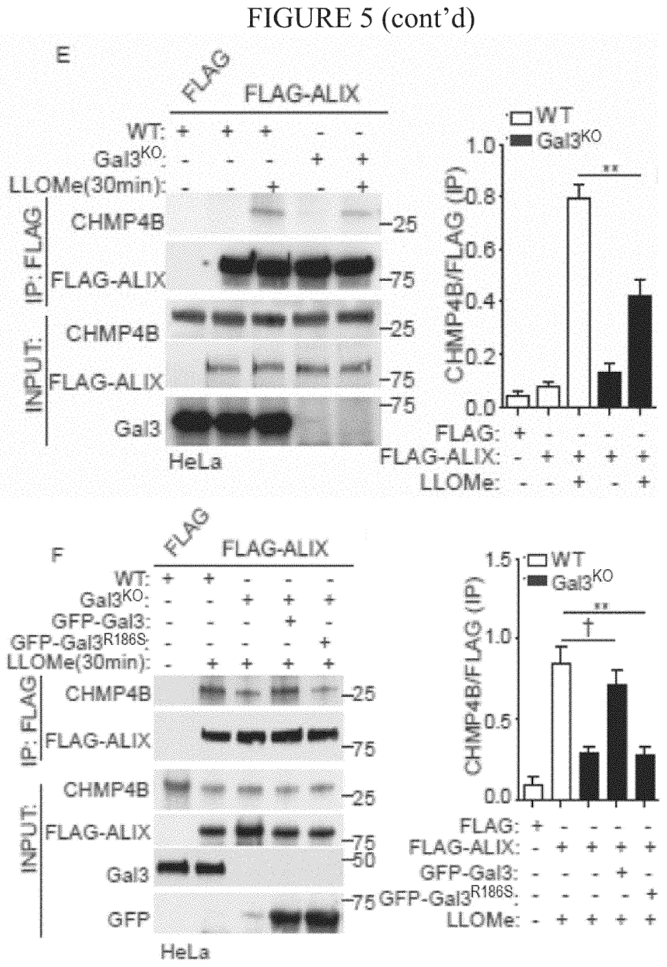

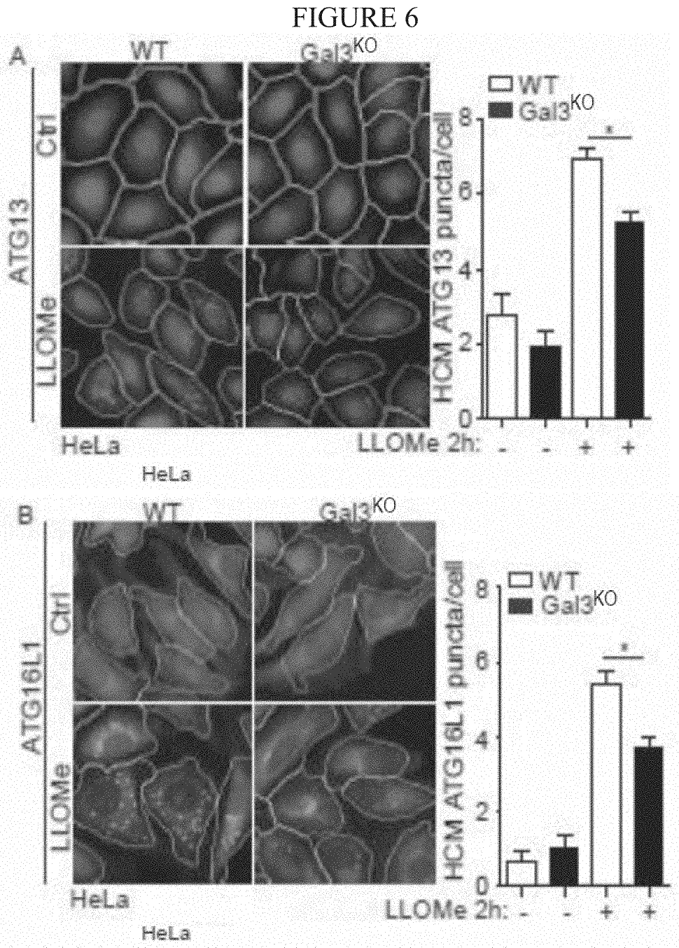

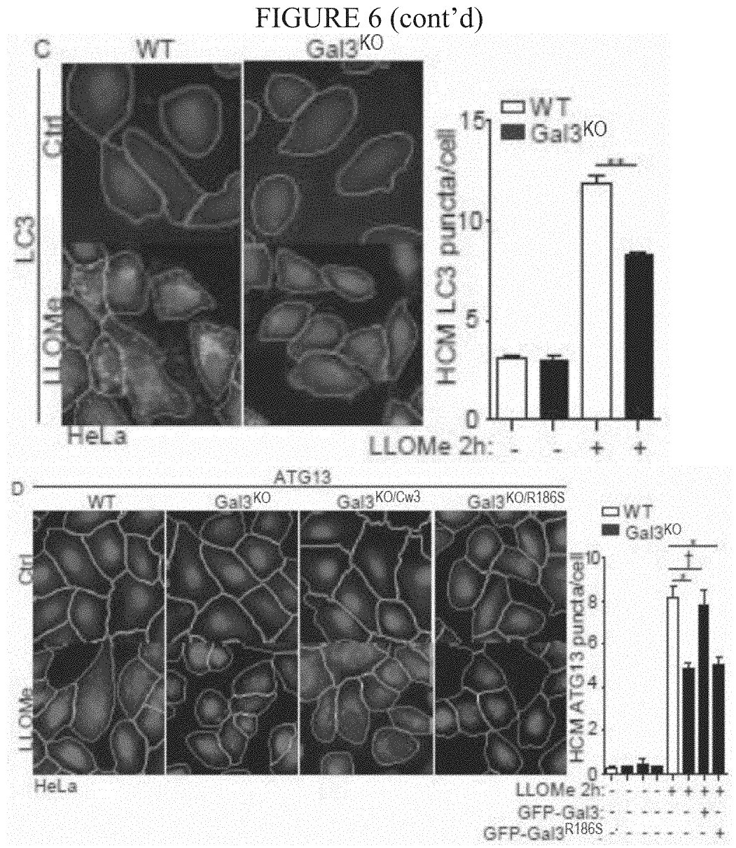

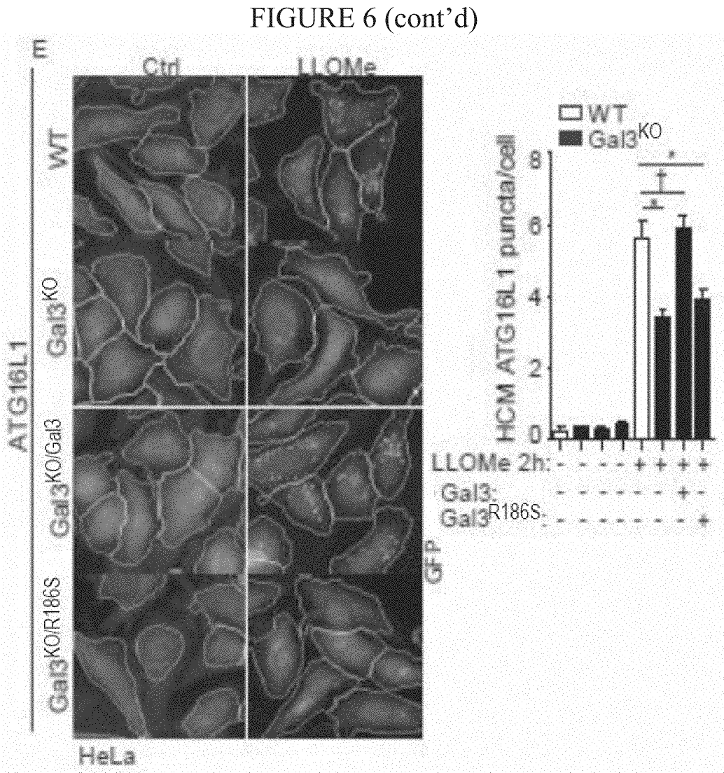

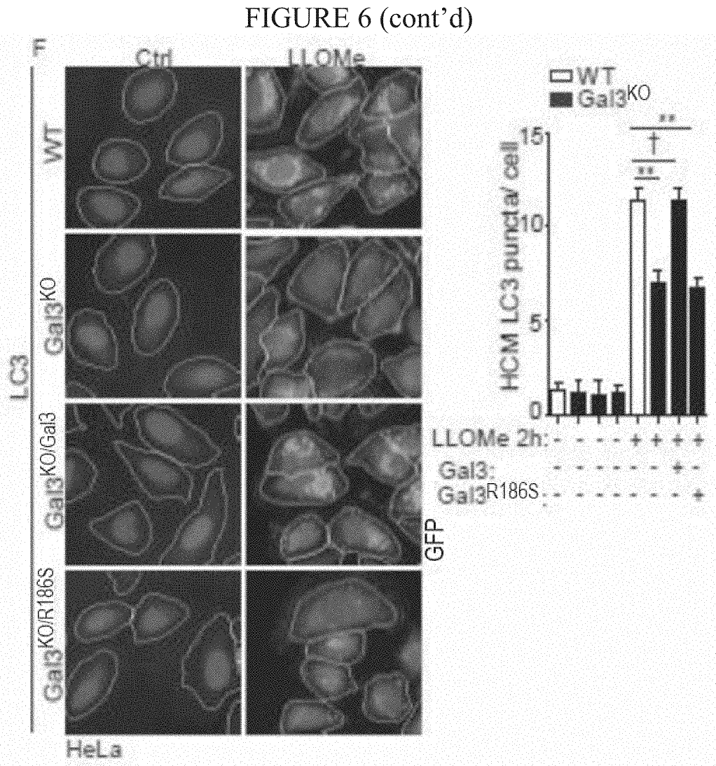

FIG. S5, related to FIGS. 5 and 6 shows that Gal3 is important for autophagy response during lysosomal damage. (A) Quantification by HCM of overlaps between mCherry-CHMP4A and endogenous LAMP1 in parental HeLa (WT) and Gal3-knockout HeLa cells (Gal3^{KO}) treated with BAPTA-AM during lysosomal damage. Cells treated with 15 μM BAPTA-AM for 1 h were subject to 1 mM LLOMe treatment for 10 min and overlaps between mCherry-CHMP4A and LAMP1 quantified by HCM analysis of Ctrl, untreated (no LLOMe) cells. White masks, algorithm-defined cell boundaries; Yellow masks, computer-identified overlap of mCherry-CHMP4A and LAMP1. (B) Mapping the interaction between Gal3 and CHMP4A. HEK293T cells overexpressing GFP-tagged full-length or truncated Gal3 and FLAG-CHMP4A were subjected to anti-GFP immunoprecipitation, followed by immunoblotting for FLAG-CHMP4A. (C) Mapping the interaction between Gal3 and CHMP4B. HEK293T cells overexpressing GFP-tagged full-length or truncated Gal3 and Myc-CHMP4B were subjected to anti-GFP immunoprecipitation, followed by immunoblotting for Myc-CHMP4B. (D) Schematic diagram of Gal3 domains, its deletion constructs and of mapping the interaction between Gal3 and CHMP4A/B or TRIM16. (E) Schematic summary of the findings in FIGS. 5 and S5. (F) Co-immunoprecipitation analysis of changes in interactions between Gal3 and TRIM16 during the process of lysosomal damage. HeLa Flp-In-Gal3^{TetON} cells stably expressing Gal3 induced by tetracycline (Tet) were treated with 1 mM LLOMe for the indicated time. Cell lysates were immunoprecipitated (IP) with anti-mCherry antibody and immunoblotted for endogenous TRIM16. (G) Mapping the interaction between Gal3 and TRIM16. HEK293T cells overexpressing GFP-tagged full-length or truncated Gal3 and FLAG-TRIM16 were subjected to anti-GFP immunoprecipitation, followed by immunoblotting for FLAG-TRIM16. (H) Schematic summary of the findings in FIGS. 6 and S6. (I) M. tuberculosis survival assay in Murine bone marrow-derived macrophages (BMMs) from TRIM16^{fl/fl}LysM-Cre⁻ and TRIM16^{fl/fl}LysM-Cre⁺ mice. BMMs were infected with M. tuberculosis strain Erdman at MOI 10 and incubated with full medium for total 18 h (−) or for 16 h in full medium plus last 2 h in EBSS (+; starvation). CFU were enumerated by plating and counting after 3 weeks of incubation. (J) Survival curves of mice infected by aerosol with virulent M. tuberculosis Erdman. Mice with a TRIM16 knockout specific for myeloid cells (TRIM16^{Fl/Fl}LysM-Cre⁺) and their TRIM16^{Fl/Fl}LysM-Cre⁻ littermates (negative for LysM-Cre) were subjected to a model of respiratory infection with M. tuberculosis. Initial lung deposition, 1,372 CFU/mouse of M. tuberculosis Erdman.

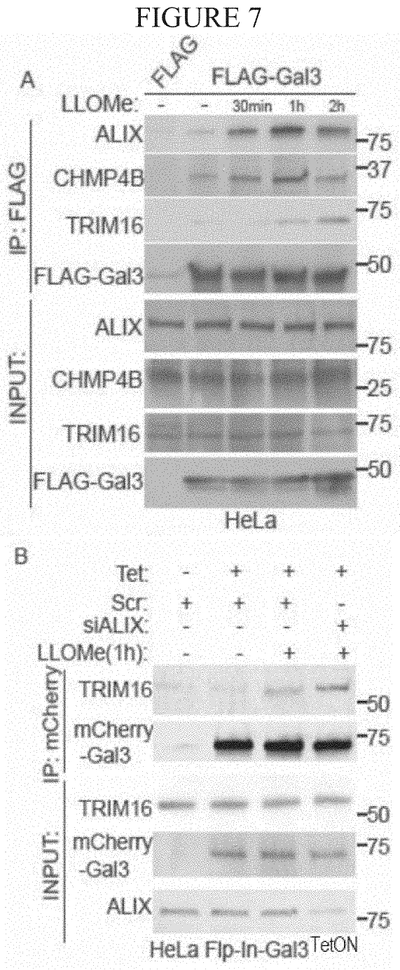

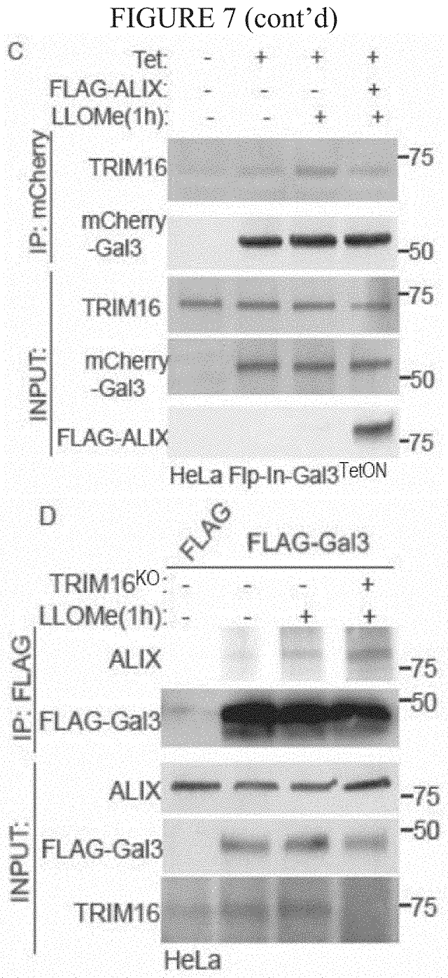

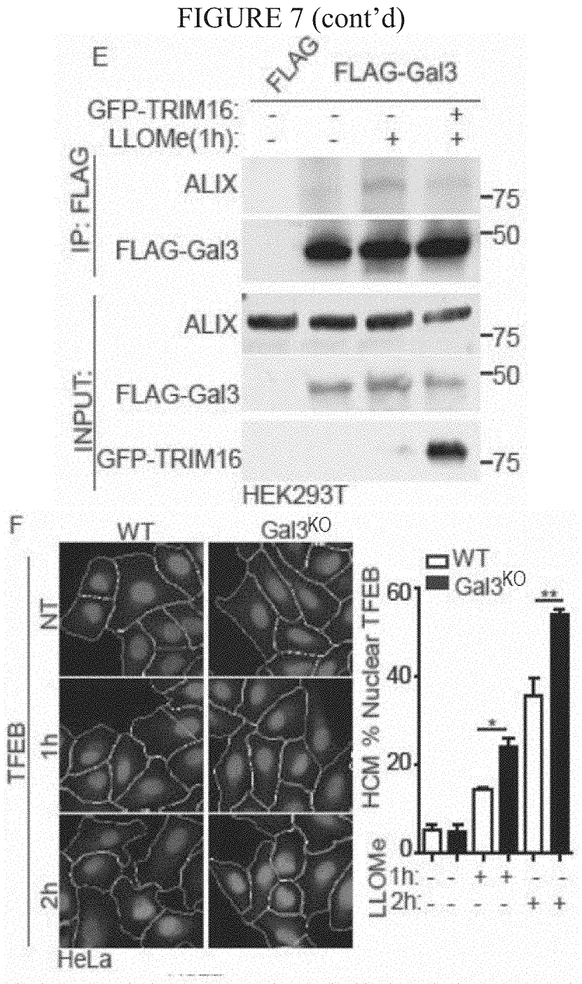

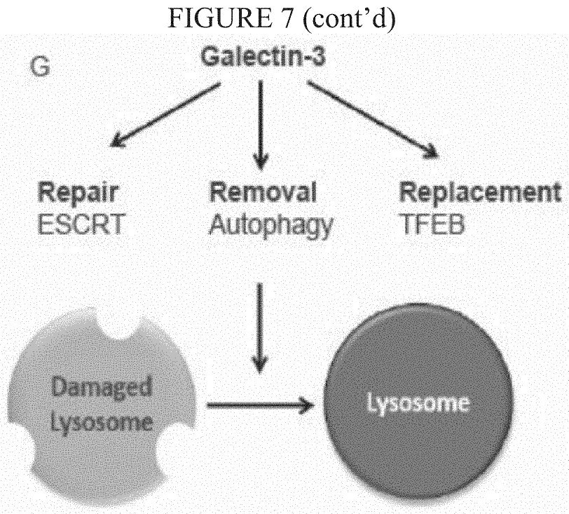

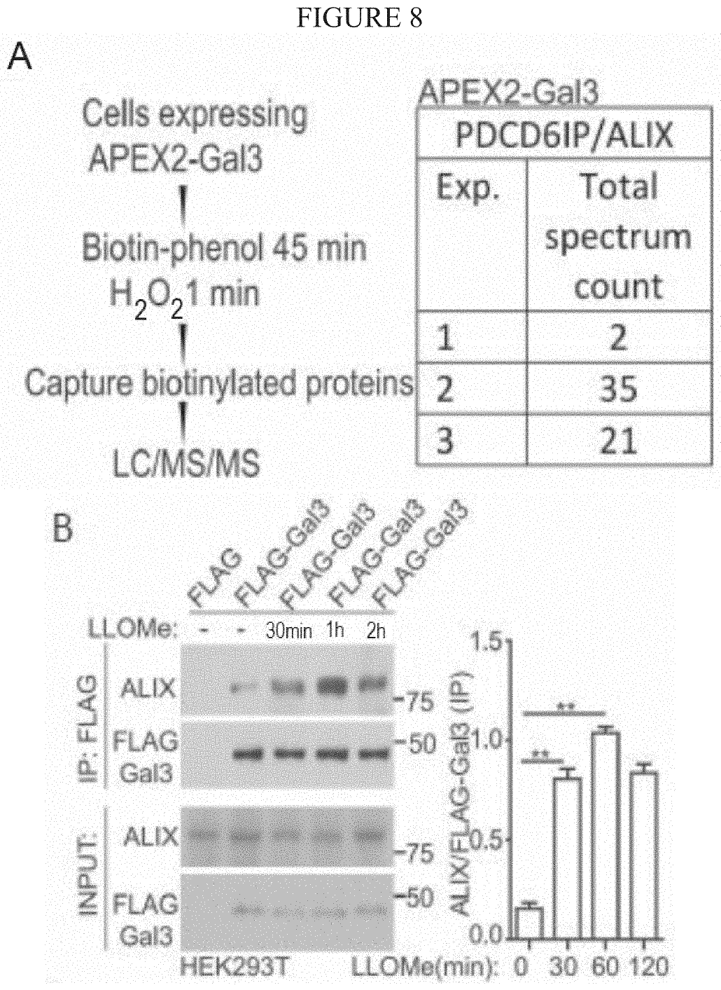

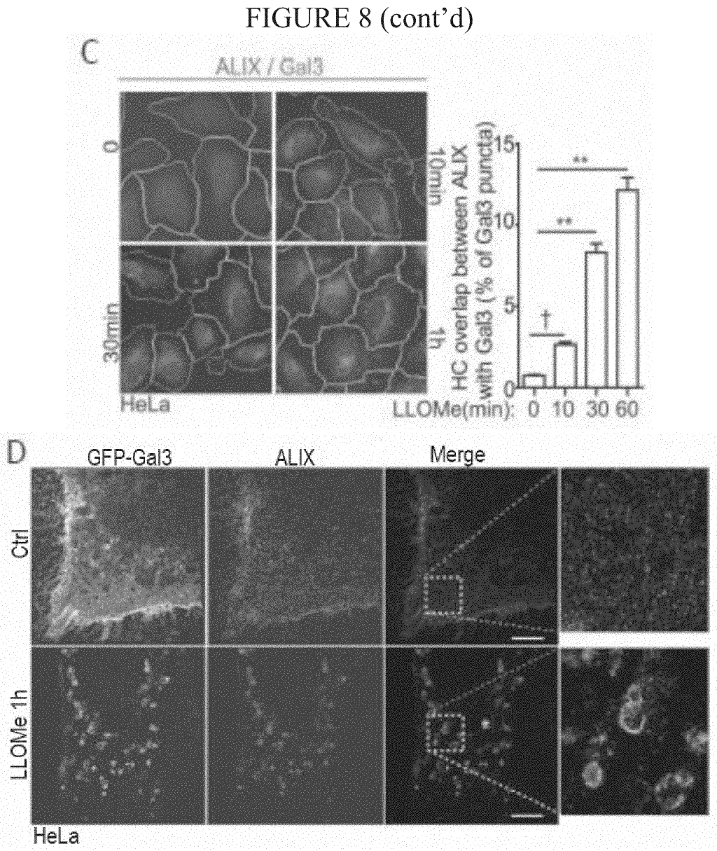

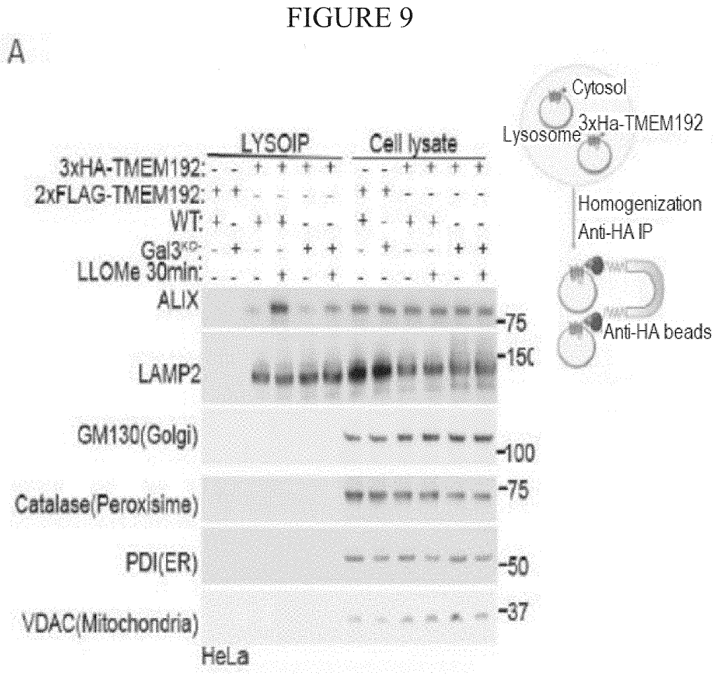

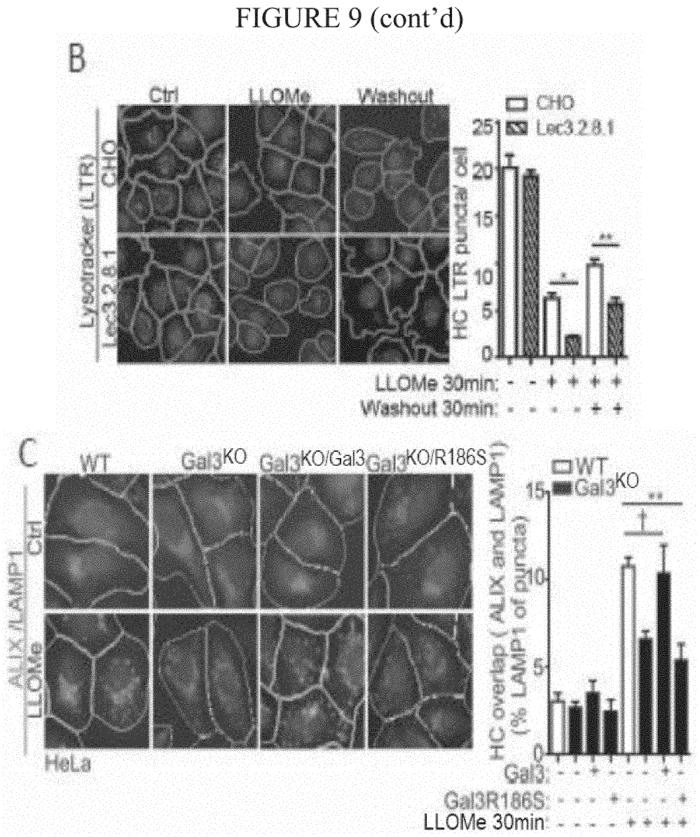

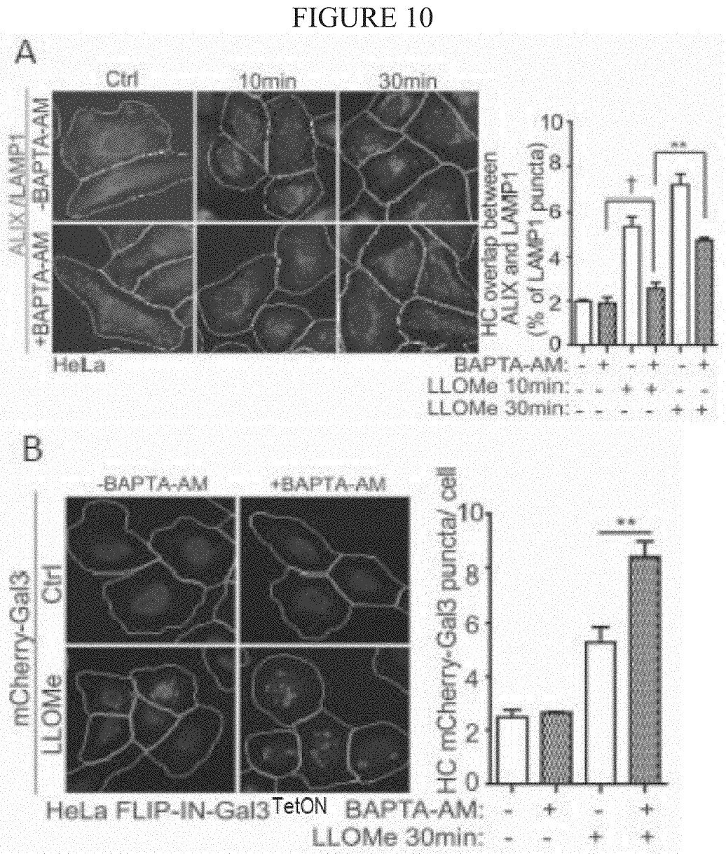

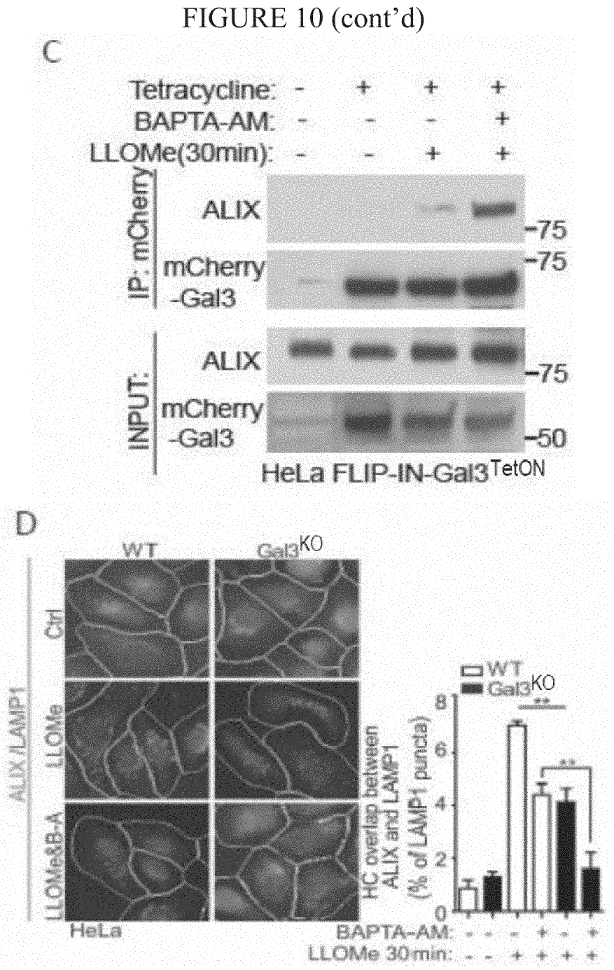

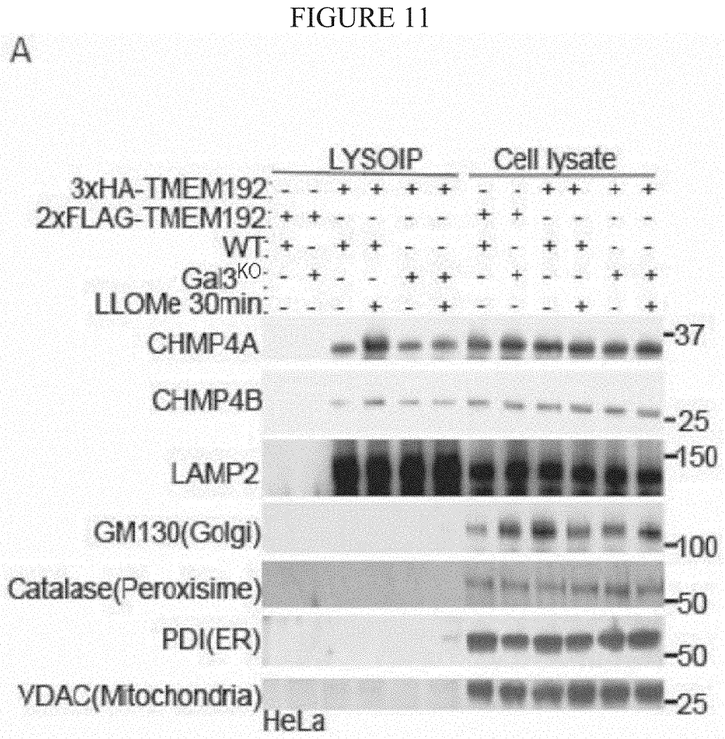

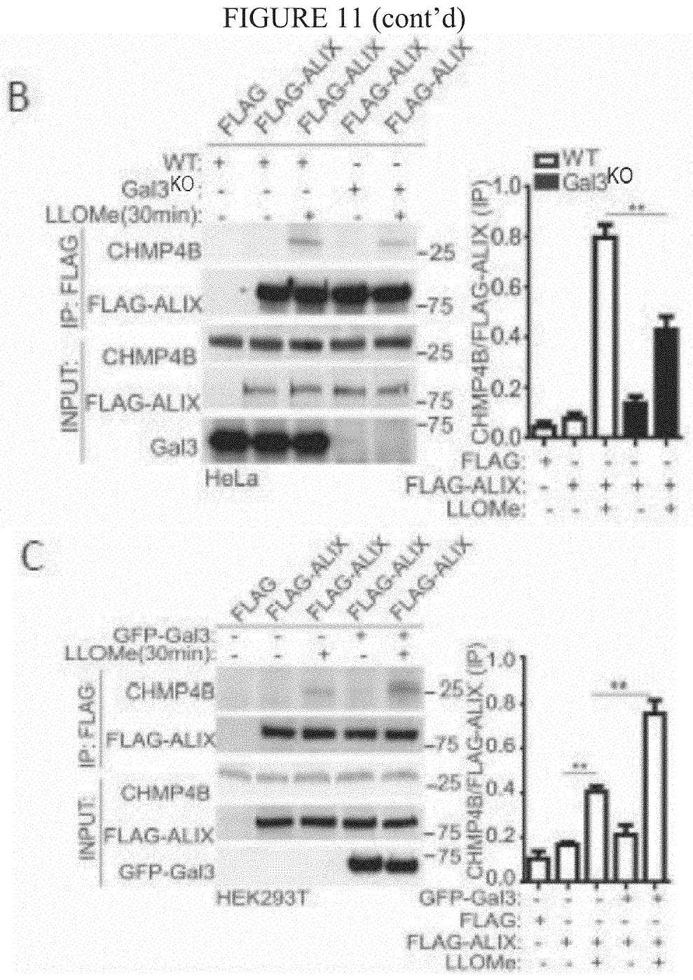

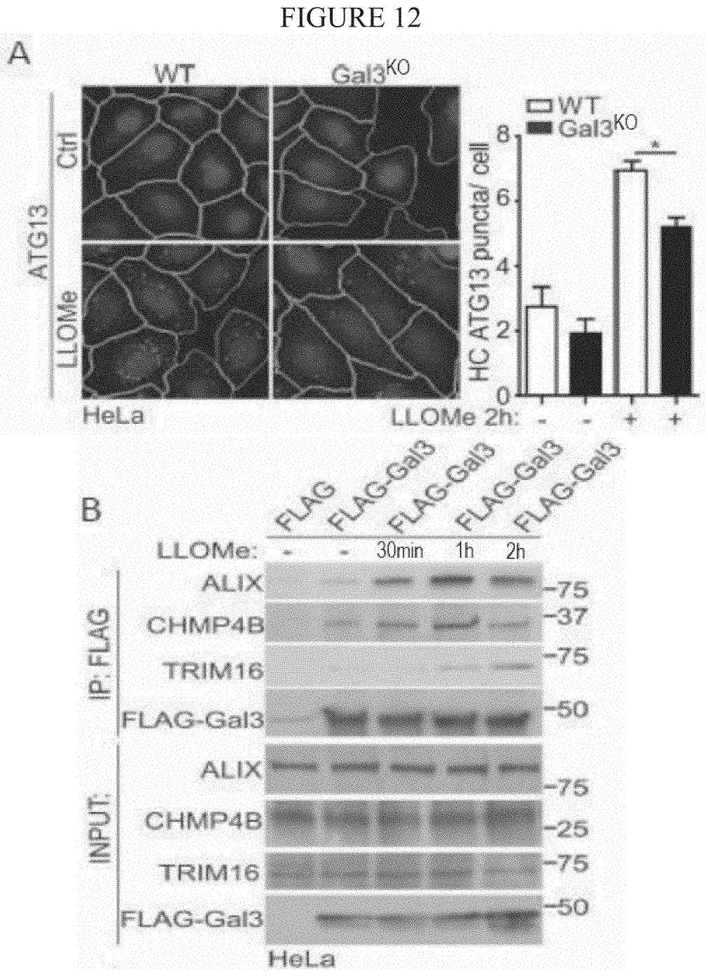

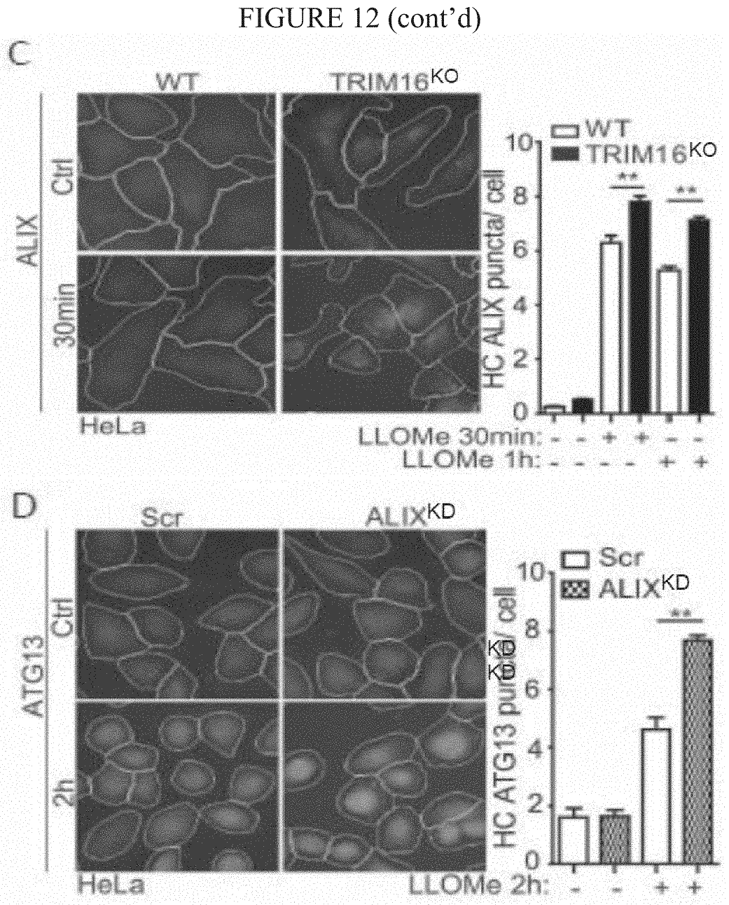

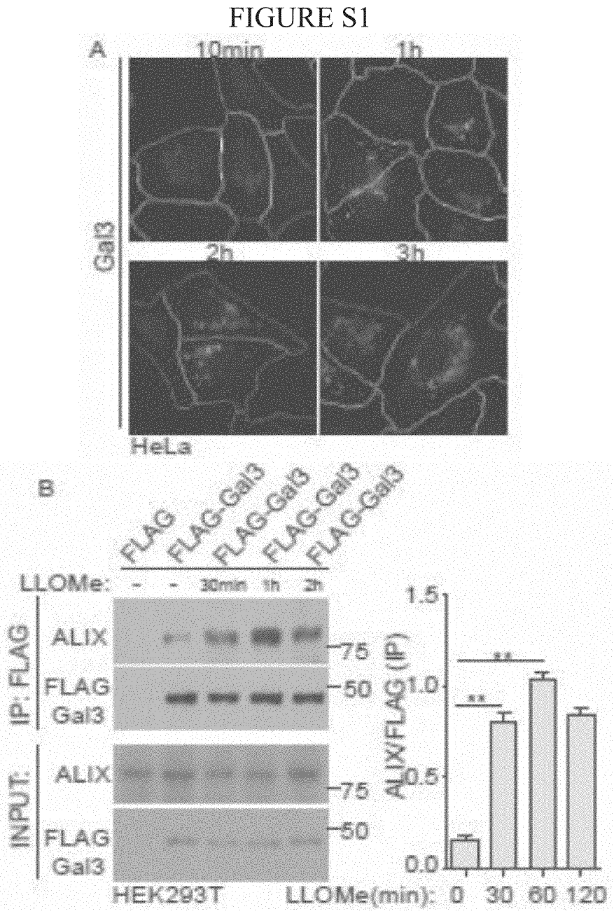

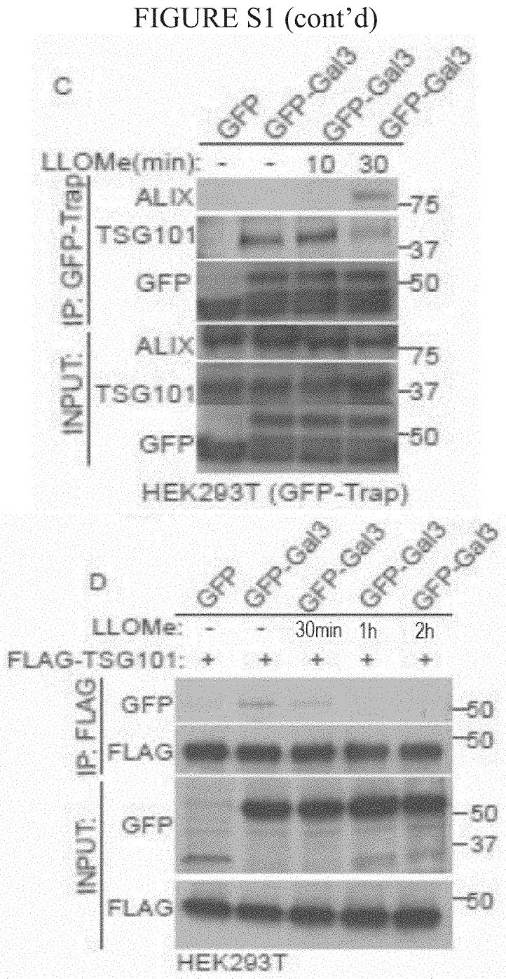

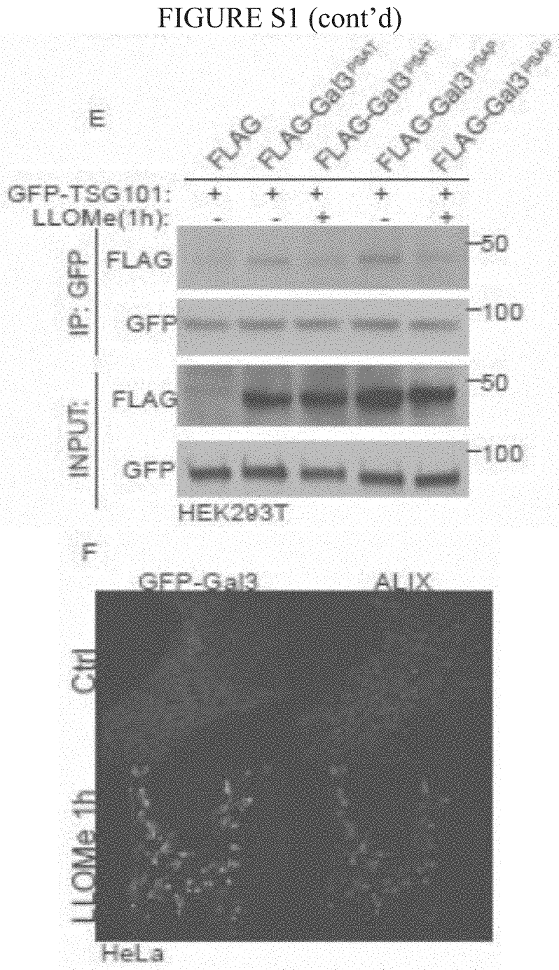

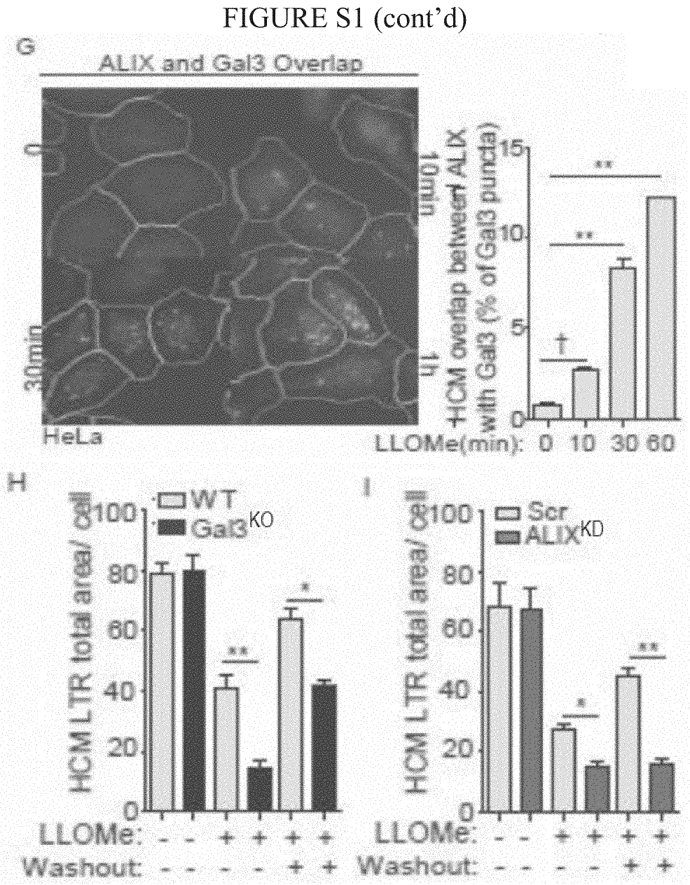

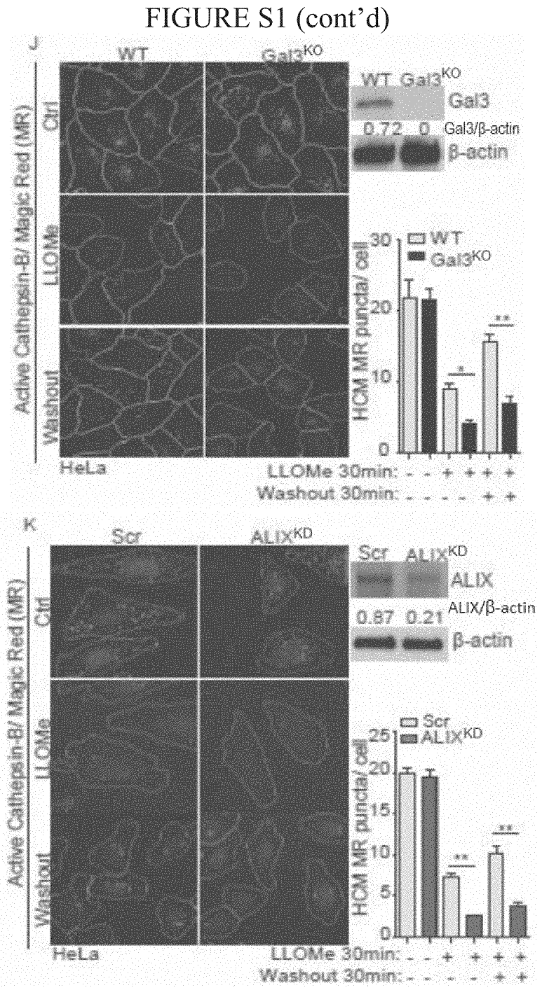

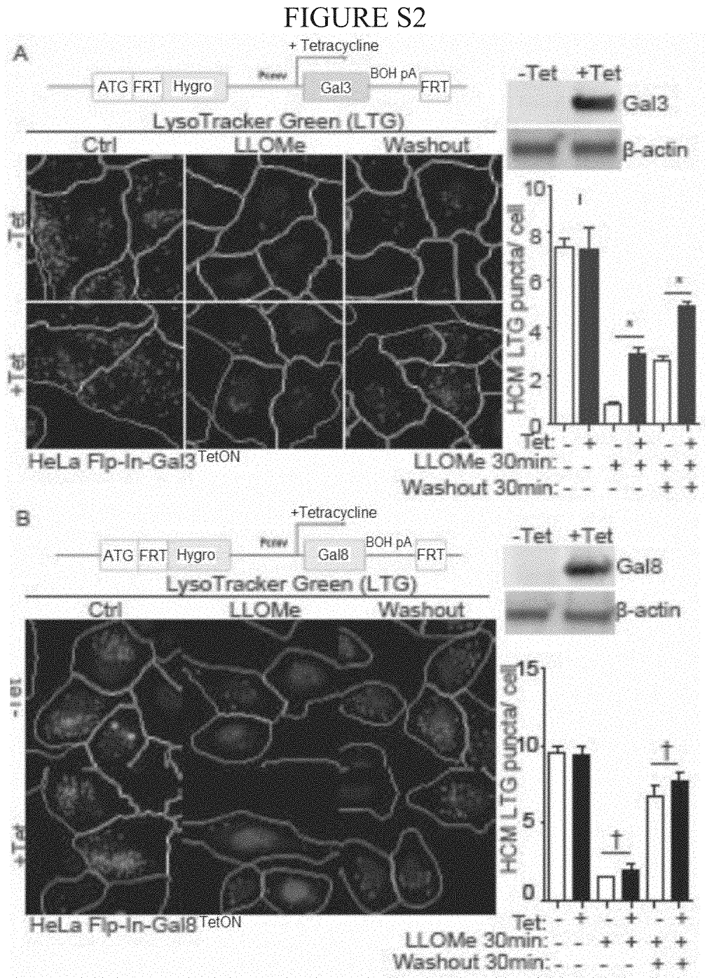

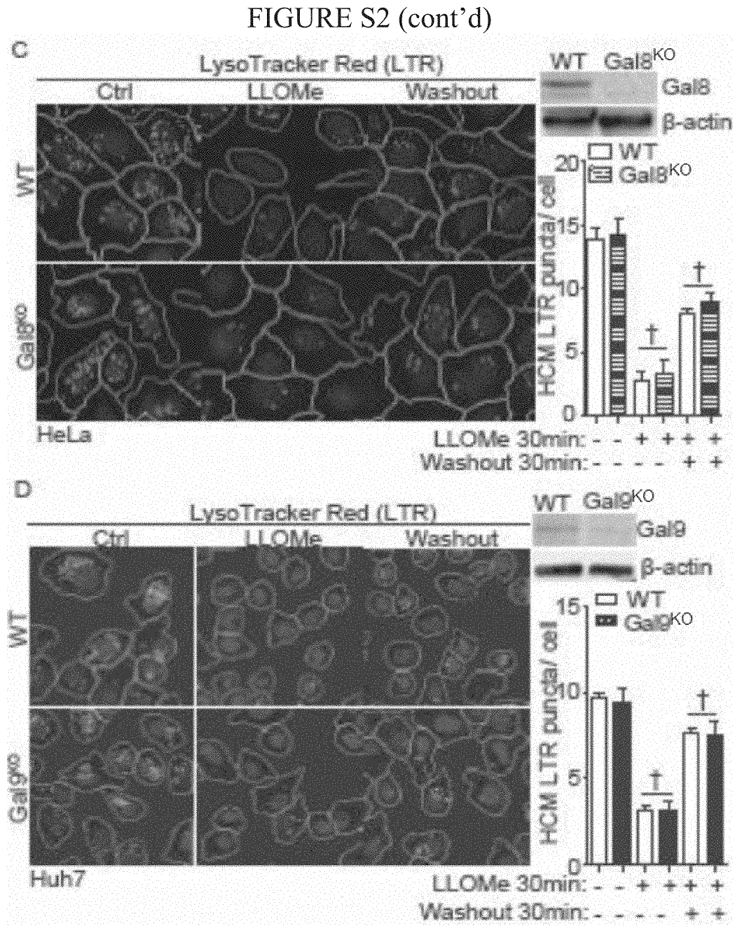

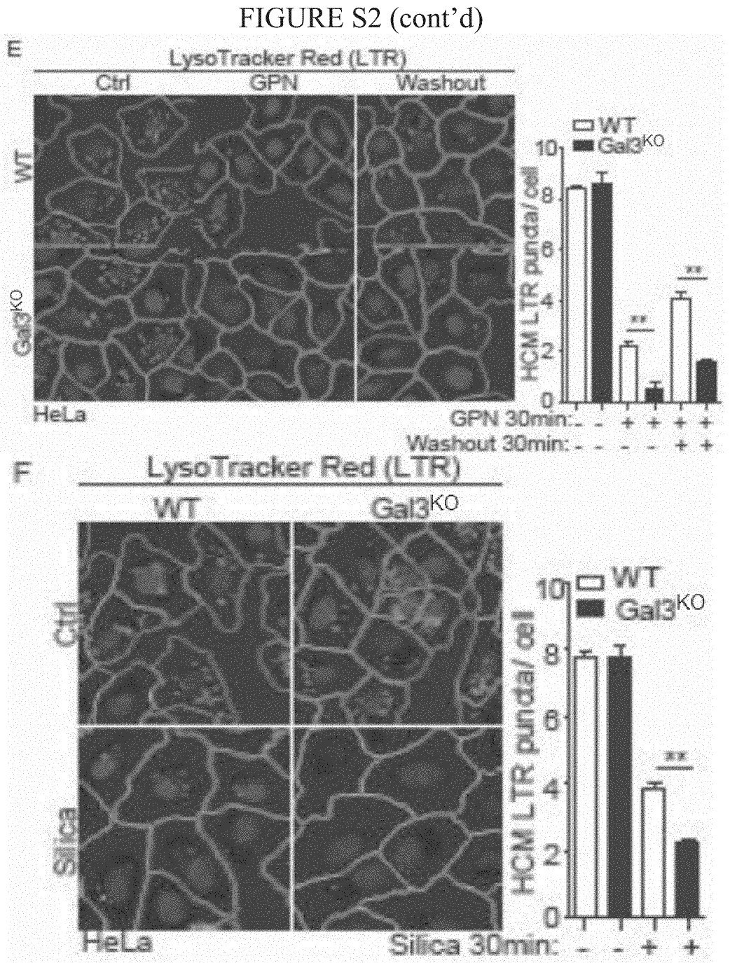

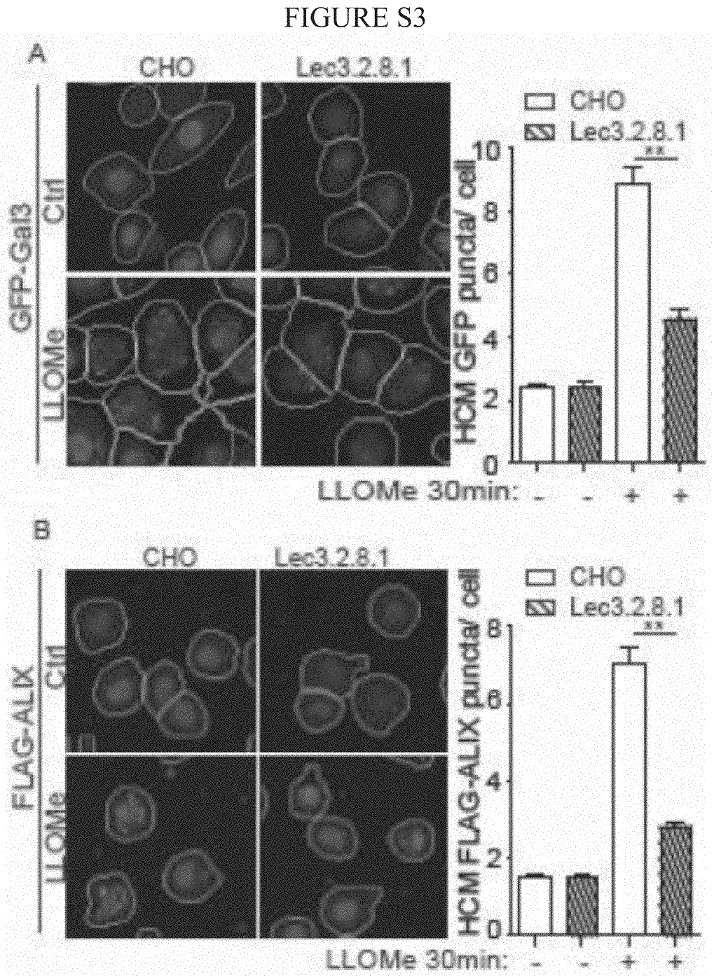

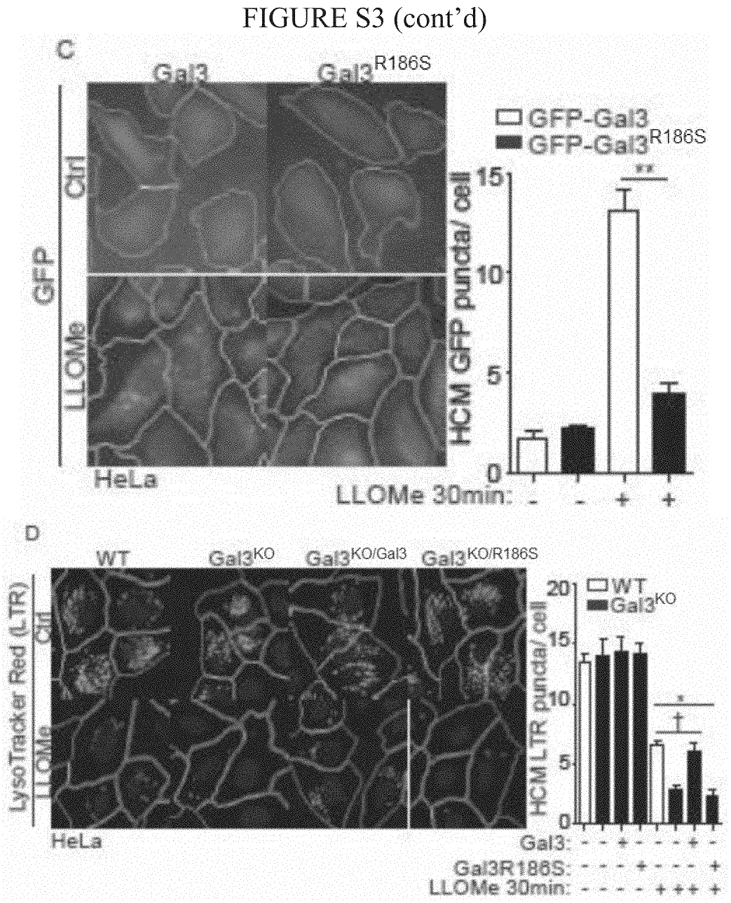

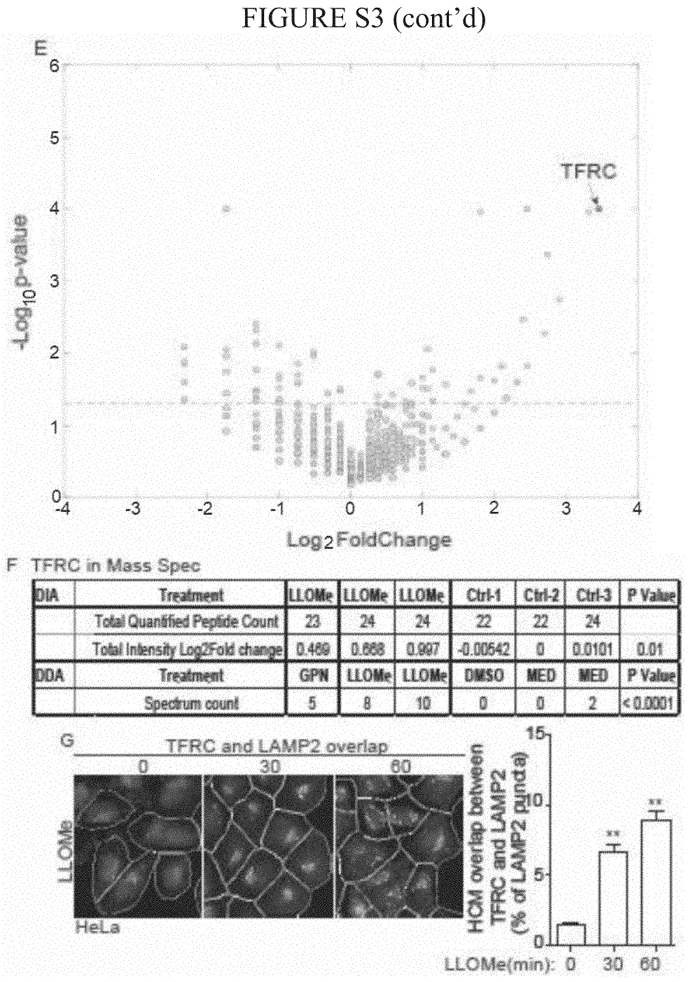

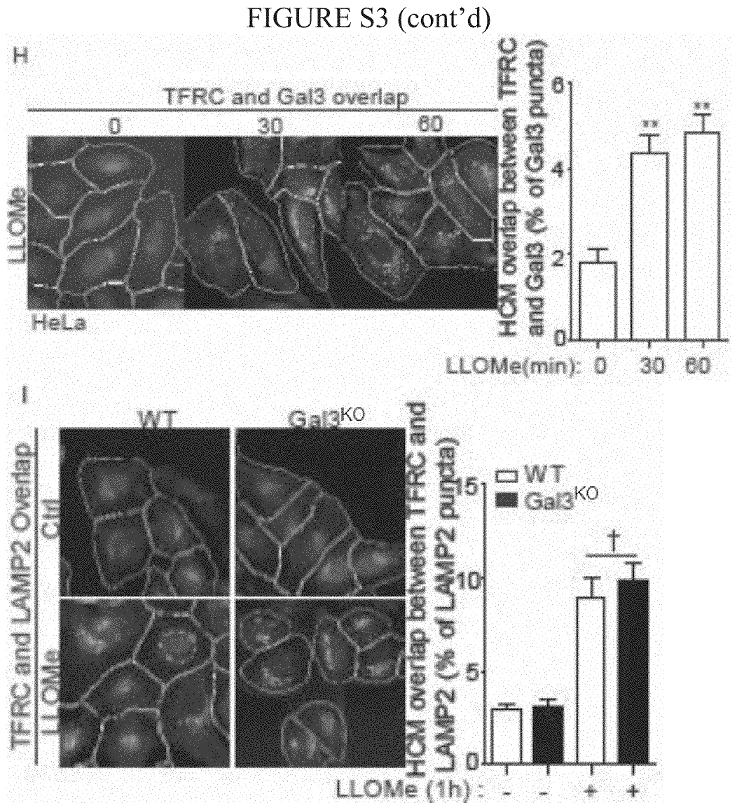

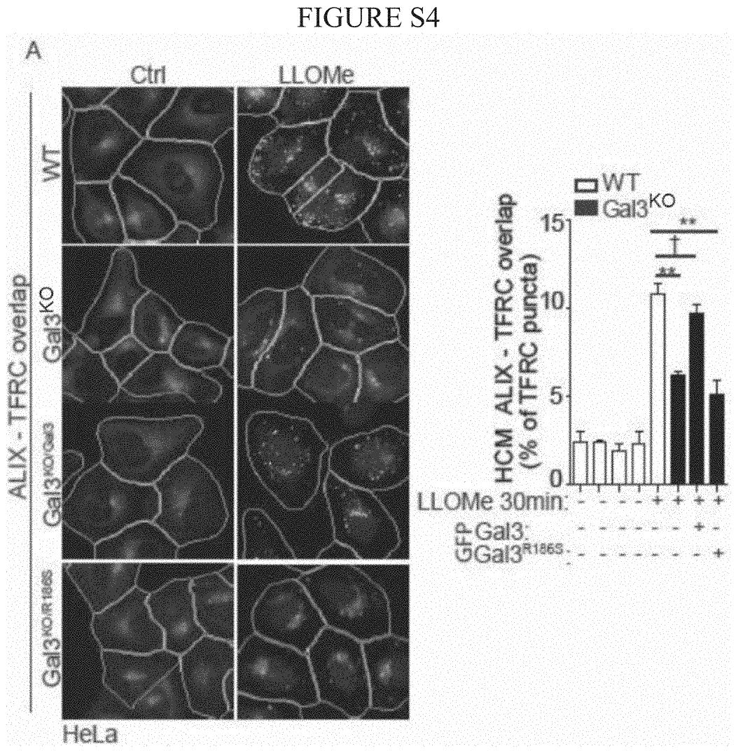

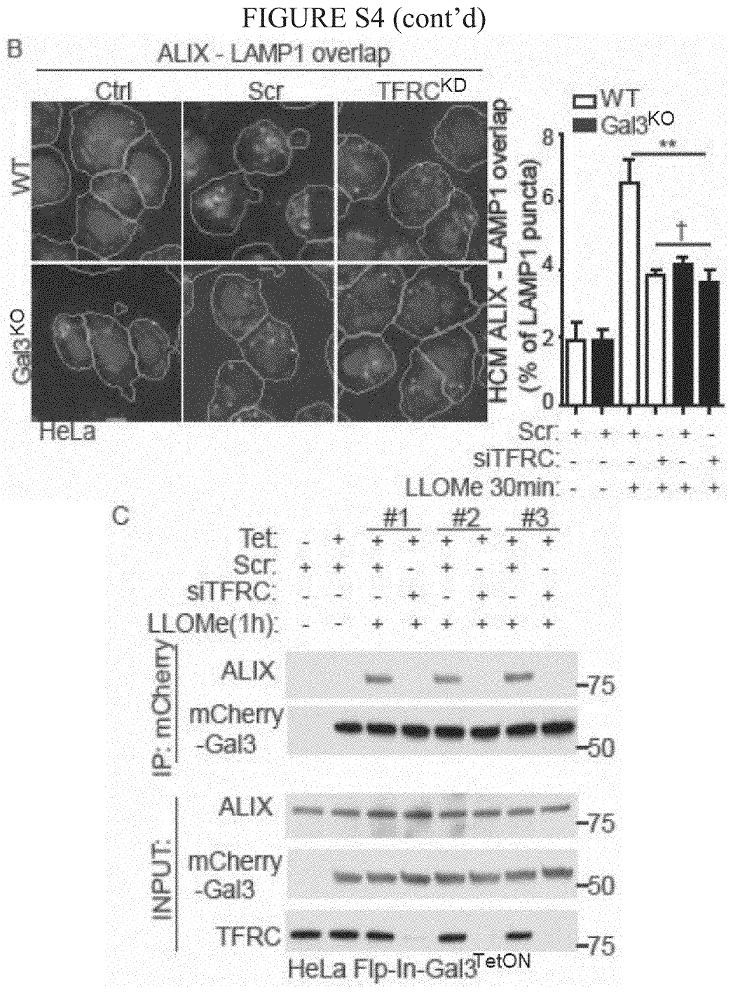

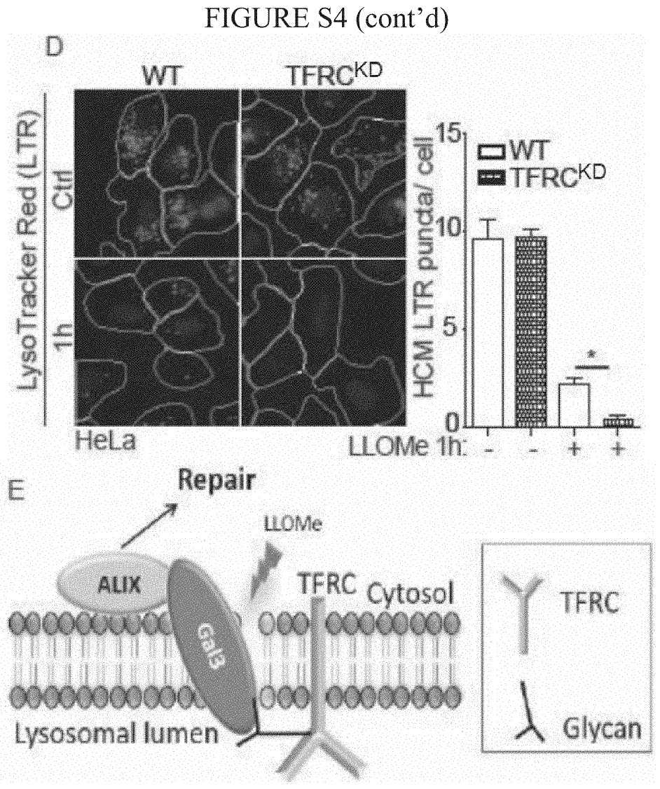

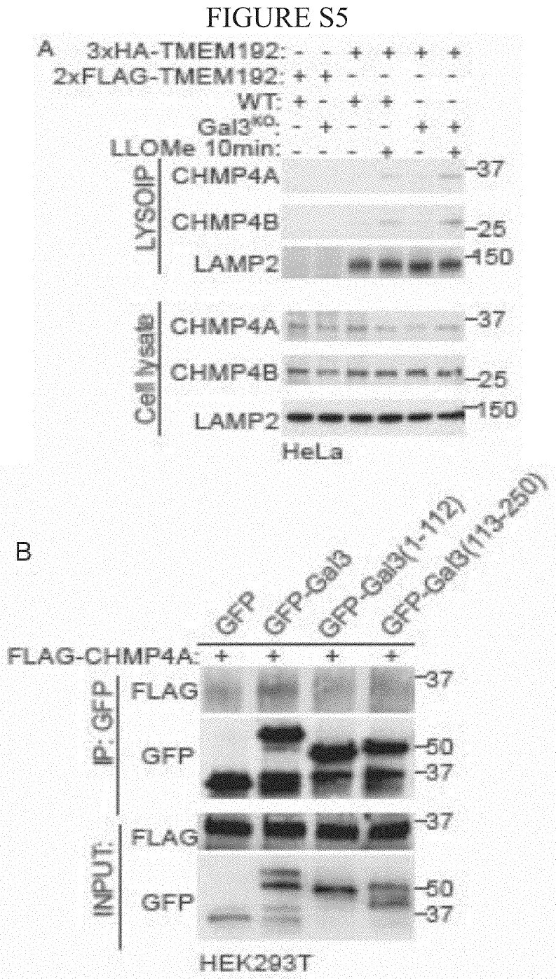

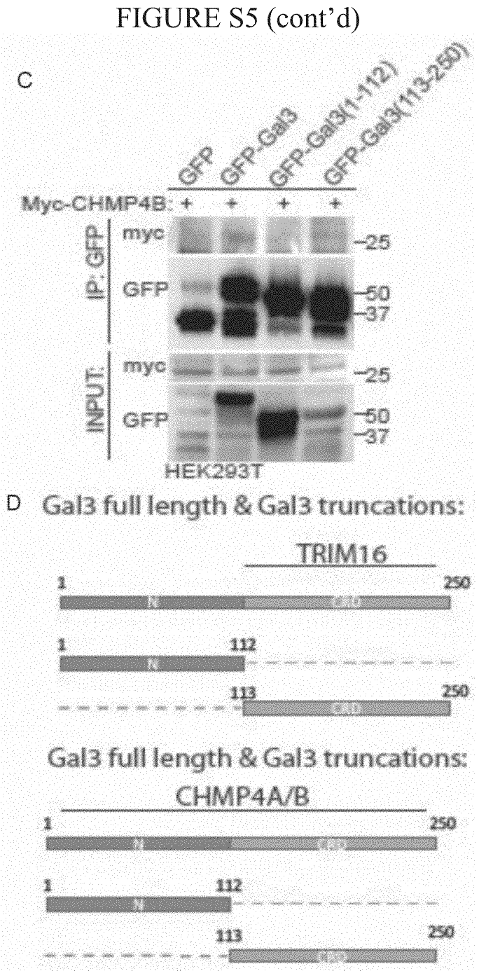

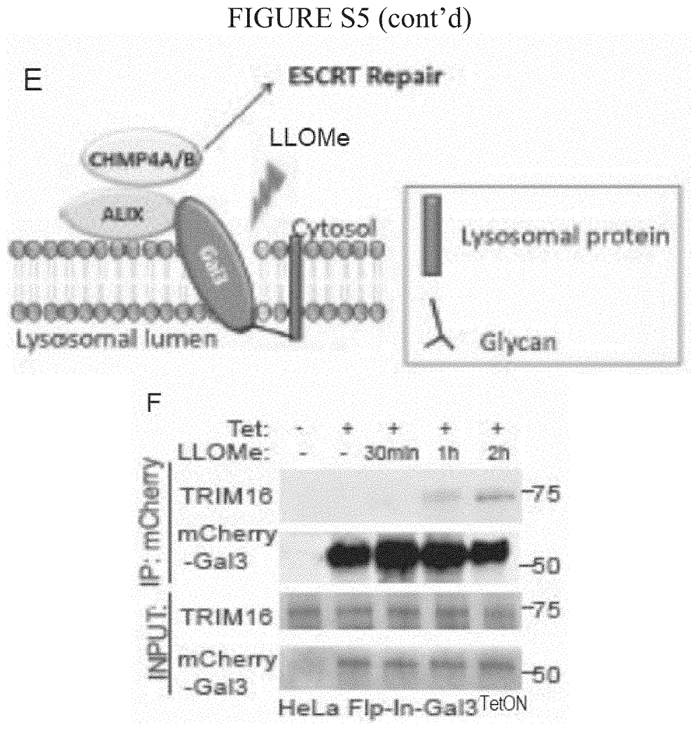

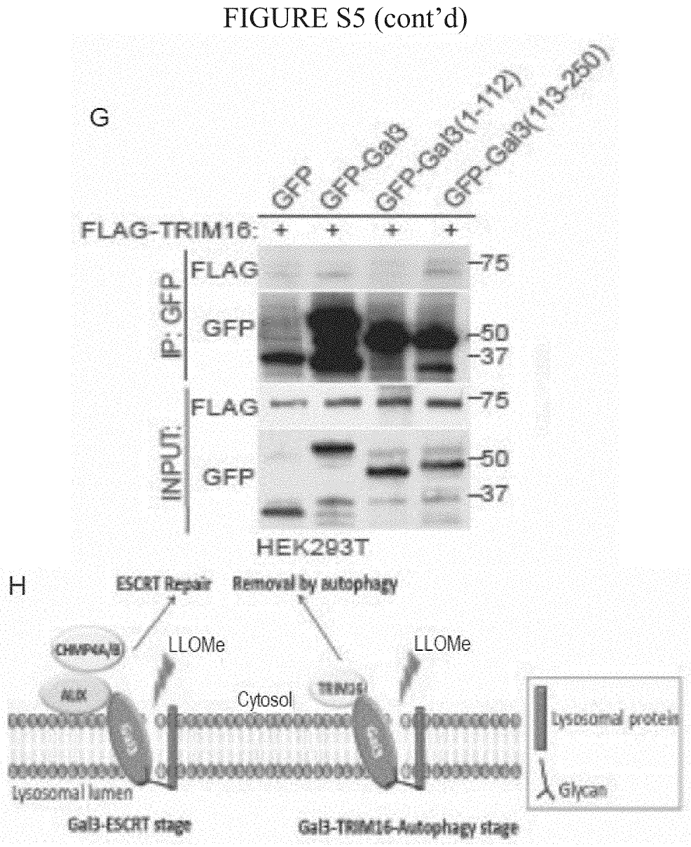

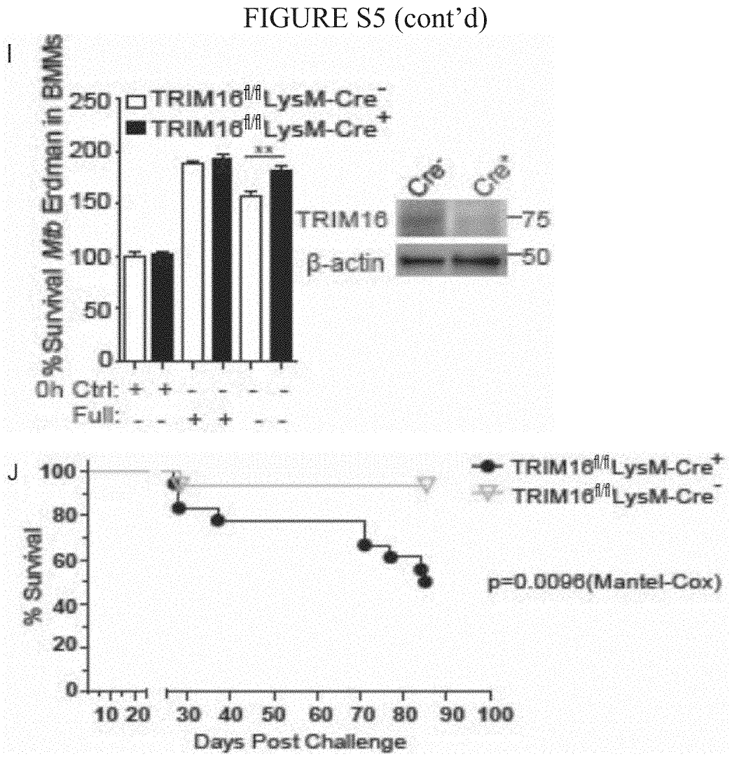

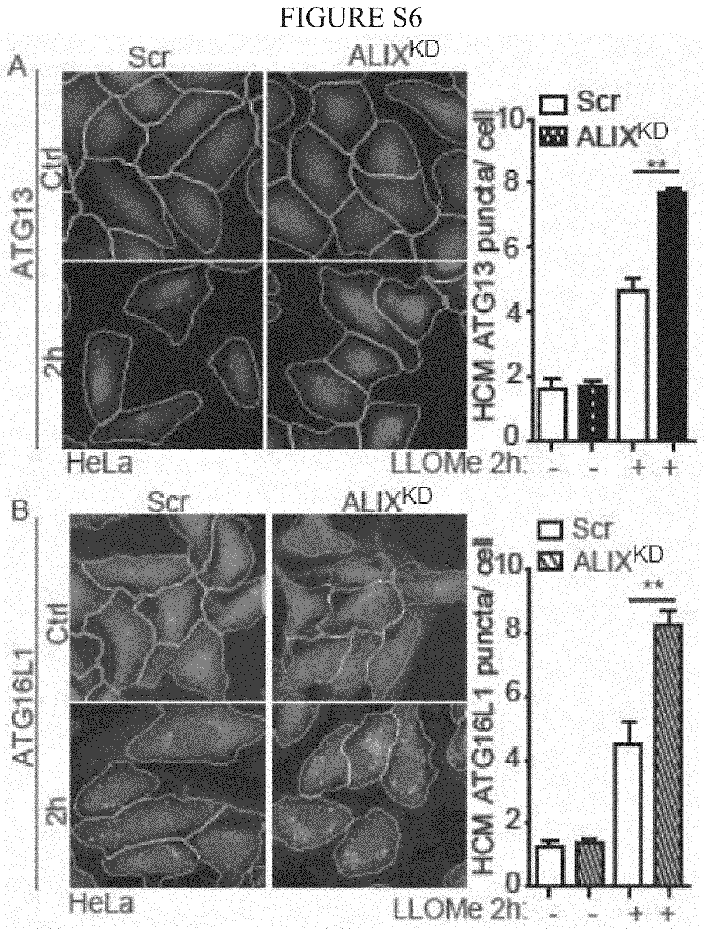

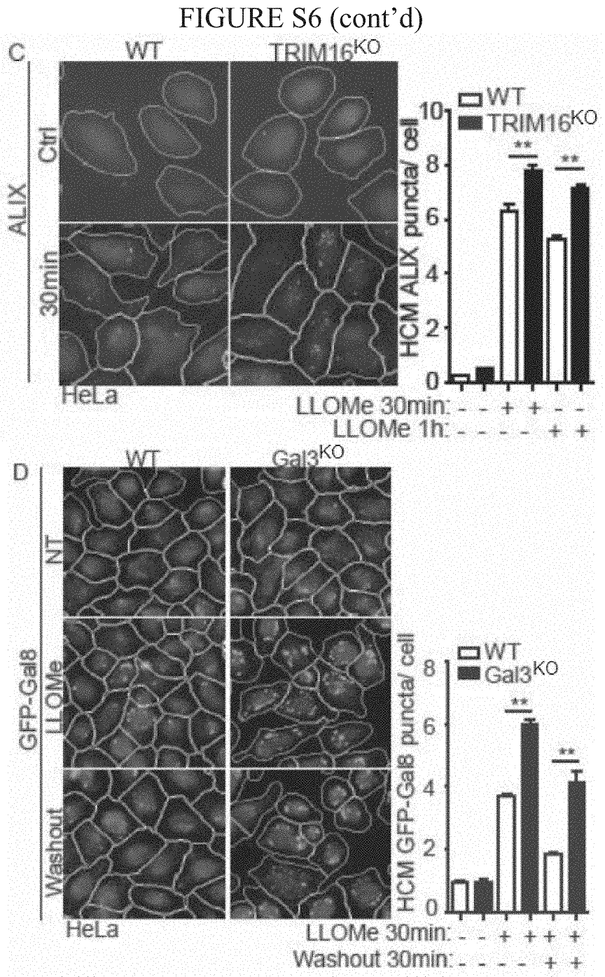

FIG. S6, related to FIG. 7 shows that Gal3 acts as a switch between repair of damaged membranes by ESCRT and their removal by autophagy. (A) ATG13 response in ALIX knockdown (ALIX^{KD}) cells during lysosomal damage. Cells transfected with scrambled siRNA as control (Scr) or ALIX siRNA were treated with 1 mM LLOMe for 2 h, and ATG13 puncta quantified by HCM. Untreated (no LLOMe) cells were used as control (Ctrl). White masks, algorithm-defined cell boundaries (primary objects); Red masks, computer-identified ATG13 puncta (target objects). (B) ATG16L1 response in ALIX knockdown (ALIX^{KD}) cells during lysosomal damage. Cells transfected with scrambled siRNA as control (Scr) or ALIX siRNA were treated with 1 mM LLOMe for 2 h, and ATG16L1 puncta were quantified using HCM. White masks, algorithm-defined cell boundaries; Green masks, computer-identified ATG16L1 puncta. (C) ALIX response in parental HeLa (WT) and TRIM16-knockout HeLa cells (TRIM16^{KO}) during lysosomal damage. HeLa cells were treated with 1 mM LLOMe for the indicated time, and ALIX puncta quantified by HCM. White masks, algorithm-defined cell boundaries; Green masks, computer-identified ALIX puncta. (D, E) Analysis of galectins' response (Gal8 (D) or Gal9 (E)) in parental HeLa (WT) and Gal3-knockout HeLa cells (Gal3^{KO}) during lysosomal damage. Cells transiently transfected with GFP-Gal8 or GFP-Gal9 were treated with 1 mM LLOMe for 30 min followed by 30 min washout, and GFP-Gal8 or GFP-Gal9 puncta quantified by HCM. White masks, algorithm-defined cell boundaries; Green masks, computer-identified GFP-Gal8/9 puncta. (F) Schematic summary of FIGS. 7 and S6.

### DETAILED DESCRIPTION OF THE INVENTION

It is noted that, as used in this specification and the appended claims, the singular forms “a,” “an,” and “the,” include plural referents unless expressly and unequivocally limited to one referent. Thus, for example, reference to “a compound” or “a modulator” includes two or more different compounds. As used herein, the term “include” and its grammatical variants are intended to be non-limiting, such that recitation of items in a list is not to the exclusion of other like items that can be substituted or other items that can be added to the listed items.

The term “compound” or “agent”, as used herein, unless otherwise indicated, refers to any specific chemical compound or composition (such as a Galectin-3 modulator, an Alix (AIP1) modulator or TFRC modulator) disclosed herein and includes tautomers, regioisomers, geometric isomers as applicable, and also where applicable, stereoisomers, including diastereomers, optical isomers (e.g. enantiomers) thereof, as well as pharmaceutically acceptable salts thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers and epimers, where applicable in context. The term also refers, in context to prodrug forms of compounds which have been modified to facilitate the administration and delivery of compounds to a site of activity.

The term “patient” or “subject” is used throughout the specification within context to describe an animal, generally a mammal, including a domesticated mammal including a farm animal (dog, cat, horse, cow, pig, sheep, goat, etc.) and preferably a human, to whom treatment, including prophylactic treatment (prophylaxis), with the methods and compositions according to the present invention is provided. For treatment of those conditions or disease states which are specific for a specific animal such as a human patient, the term patient refers to that specific animal, often a human.

The terms “effective” or “pharmaceutically effective” are used herein, unless otherwise indicated, to describe an amount of a compound or composition which, in context, is used to produce or affect an intended result, usually the modulation (inhibition or upregulation) of autophagy within the context of a particular treatment or alternatively, the effect of a bioactive agent which is coadministered with the autophagy modulator (autotoxin) in the treatment of disease.

The terms “treat”, “treating”, and “treatment”, etc., as used herein, refer to any action providing a benefit to a patient at risk for or afflicted by an autophagy mediated disease state or condition as otherwise described herein, especially where excessive inflammation results from the disease state and/or condition. The benefit may be in curing the disease state or condition, inhibiting its progression, or ameliorating, lessening or suppressing one or more symptom of an autophagy mediated disease state or condition, especially including excessive inflammation caused by the disease state and/or condition. Treatment, as used herein, encompasses therapeutic treatment and in certain instances, prophylactic treatment, depending on context.

The term “Galectin-3”, “Galectin protein-3”, “Gal3” or “GAL-3” is used to describe a protein defined by its binding specificity for β-galactoside sugars, such as N-acetyllactosamine (Galβ1-3GlcNAc or Galβ1-4GlcNAc), which can be bound to proteins by either N-linked or O-linked glycosylation. Galectin-3 is a lectin with a specific affinity for β-galactoside glycoconjugates, is an approximately 30-kDa signaling protein and, like all galectins, contains a carbohydrate recognition domain of approximately 130 amino acids that enables the specific binding of β-galactosides.

In preferred aspects of the invention, human Galectin-3 is referred to. As used herein, the term Galectin-3 describes Galectin-3 protein and includes variants thereof having at least 80%, 85%, 90% or 95% sequence identity to the most common form of the protein, which is preferably the human protein. Preferred Galectin-3 proteins include the following Galectin proteins or variants thereof having at least 80%, 85%, 90% or 95% sequence identity. The following sequences are representative of the human Galectin proteins which are of preferred use as a target of GAL-3 modulators herein.

Galectin-3 Accession No. NP_002297.2 SEQ ID NO: 1

The term “Alix”, “AIP1”, “Alix (AIP1)” “ALG-2 interacting protein 1” refers to a protein which is believed to participate in programmed cell death. Studies in mouse cells have shown that overexpression of this protein can block apoptosis. In addition, the product of this gene binds to the product of the PDCD6 gene, a protein required for apoptosis, in a calcium-dependent manner. This gene product also binds to endophilins, proteins that regulate membrane shape during endocytosis. Overexpression of this gene product and endophilins may be partly responsible for the protection against cell death.

The term “Transferrin Receptor” “TFRC” or “Transferrin Receptor Protein 1” is used to refer to the transferrin receptor protein. The transferrin receptor is a membrane glycoprotein whose function is to mediate cellular uptake of iron from a plasma glycoprotein, transferrin. Iron uptake from transferrin involves the binding of transferrin to the transferrin receptor, internalization of transferrin within an endocytic vesicle by receptor-mediated endocytosis and the release of iron from the protein by a decrease in endosomal pH. With the exception of highly differentiated cells, transferrin receptors are probably expressed on all cells but their levels vary greatly. Transferrin receptors are highly expressed on immature erythroid cells, placental tissue, and rapidly dividing cells, both normal and malignant.

As used herein, the term “autophagy mediated disease state or condition” refers to a disease state or condition that results from disruption in autophagy or cellular self-digestion and in particular, causes or is a risk for causing excessive inflammation. Autophagy is a cellular pathway involved in protein and organelle degradation, and has a large number of connections to human disease. Autophagic dysfunction which causes inflammation is associated with inflammatory diseases, including neurodegeneration, autoimmune diseases, microbial infections, cardiovascular diseases and metabolic diseases including diabetes mellitus, among numerous other disease states and/or conditions. Although autophagy plays a principal role as a protective process for the cell, it also plays a role in cell death. Disease states and/or conditions which are mediated through autophagy (which refers to the fact that the disease state or condition may manifest itself as a function of the increase or decrease in autophagy in the patient or subject to be treated and treatment requires administration of an inhibitor or agonist of autophagy in the patient or subject) include, for example, lysosomal storage diseases (discussed hereinbelow), neurodegeneration (including, for example, Alzheimer's disease, Parkinson's disease, Huntington's disease; other ataxias), immune response (T cell maturation, B cell and T cell homeostasis, counters damaging inflammation), autoimmune diseases and chronic inflammatory diseases resulting in excessive inflammation (these disease states may promote excessive cytokines when autophagy is defective), including, for example, inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease; hyperglycemic disorders, diabetes (I and II), affecting lipid metabolism islet function and/or structure, excessive autophagy may lead to pancreatic β-cell death and related hyperglycemic disorders, including severe insulin resistance, hyperinsulinemia, insulin-resistant diabetes (e.g. Mendenhall's Syndrome, Werner Syndrome, leprechaunism, and lipoatrophic diabetes) and dyslipidemia (e.g. hyperlipidemia as expressed by obese subjects, elevated low-density lipoprotein (LDL), depressed high-density lipoprotein (HDL), and elevated triglycerides) and metabolic syndrome, liver disease (excessive autophagic removal of cellular entities—endoplasmic reticulum), renal disease (apoptosis in plaques, glomerular disease), cardiovascular disease (especially including infarction, ischemia, stroke, pressure overload and complications during reperfusion), muscle degeneration and atrophy, symptoms of aging (including amelioration or the delay in onset or severity or frequency of aging-related symptoms and chronic conditions including muscle atrophy, frailty, metabolic disorders, low grade inflammation, gout, silicosis, atherosclerosis and associated conditions such as cardiac and neurological both central and peripheral manifestations including stroke, age-associated dementia and sporadic form of Alzheimer's disease, and psychiatric conditions including depression), stroke and spinal cord injury, arteriosclerosis, infectious diseases (microbial infections, removes microbes, provides a protective inflammatory response to microbial products, limits adaptation of autophagy of host by microbe for enhancement of microbial growth, regulation of innate immunity) including bacterial, fungal, cellular and viral (including secondary disease states or conditions associated with infectious diseases especially including Mycobacterial infections such as M. tuberculosis, and viral infections such as heptatis B and C and HIV I and II), including AIDS, among others. Many of these diseases and/or conditions respond favorably to agonists of GAL-3, Alix (AIPI) and/or TFRC as described herein.

In addition, an autophagy disease state or condition includes autoimmune diseases such as myocarditis, Anti-glomercular Base Membrane Nephritis, lupus erythematosus, lupus nephritis, autoimmune hepatitis, primary biliary cirrhosis, alopecia areata, autoimmune urticaria, bullous pemphagoid, dermatitis herpetiformis, epidermolysis bullosa acquisita, linear IgA disease (LAD), pemphigus vulgaris, psoriasis, Addison's disease, autoimmune polyendocrine syndrome I, II and III (APS I, APS II, APS III), autoimmune pancreatitis, type I diabetes, autoimmune thyroiditis, Ord's thyroiditis, Grave's disease, autoimmune oophoritis, Sjogren's syndrome, autoimmune enteropathy, Coeliac disease, Crohn's disease, autoimmune hemolytic anemia, autoimmune lymphoproliferative syndrome, autoimmune neutropenia, autoimmune thrombocytopenic purpura, Cold agglutinin disease, Evans syndrome, pernicious anemia, Adult-onset Still's disease, Felty syndrome, juvenile arthritis, psoriatic arthritis, relapsing polychondritis, rheumatic fever, rheumatoid arthritis, myasthenia gravis, acute disseminated encephalomyelitis (ADEM), balo concentric sclerosis, Guillain-Barré syndrome, Hashimoto's encephalopathy, chronic inflammatory demvelinating polyneuropathy, Lambert-Eaton myasthenic syndrome, multiple sclerosis, autoimmune uveitis, Graves opthalmopathy, Granulomatosis with polyangitis (GPA), Kawasaki's disease, vasculitis and chronic fatigue syndrome, among others.

The term “lysosomal storage disorder” refers to an autophagy mediated disease state or condition that results from a defect in lysosomomal storage. These disease states or conditions generally occur when the lysosome malfunctions. Lysosomal storage disorders are caused by lysosomal dysfunction usually as a consequence of deficiency of a single enzyme required for the metabolism of lipids, glycoproteins or mucopolysaccharides. The incidence of lysosomal storage disorder (collectively) occurs at an incidence of about about 1:5,000-1:10,000. The lysosome is commonly referred to as the cell's recycling center because it processes unwanted material into substances that the cell can utilize. Lysosomes break down this unwanted matter via high specialized enzymes. Lysosomal disorders generally are triggered when a particular enzyme exists in too small an amount or is missing altogether. When this happens, substances accumulate in the cell. In other words, when the lysosome doesn't function normally, excess products destined for breakdown and recycling are stored in the cell. Lysosomal storage disorders are genetic diseases, but these may be treated using autophagy modulators according to the present invention, especially where the disease state or condition produces excessive inflammation as otherwise described herein. All of these diseases share a common biochemical characteristic, i.e., that all lysosomal disorders originate from an abnormal accumulation of substances inside the lysosome. Lysosomal storage diseases mostly affect children who often die as a consequence at an early stage of life, many within a few months or years of birth. Many other children die of this disease following years of suffering from various symptoms of their particular disorder.

Examples of lysosomal storage diseases include, for example, activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM2 Ganliosidosis, including infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome.,Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs and Wolnian disease, among others.

The term “modulator of autophagy”, “regulator of autophagy” or “autostatin” is used to refer to a compound which functions as an agonist (inducer or up-regulator) or antagonist (inhibitor or down-regulator) of autophagy. Depending upon the disease state or condition, autophagy may be upregulated (and require inhibition of autophagy for therapeutic intervention) or down-regulated (and require upregulation of autophagy for therapeutic intervention). In most instances, in the case of cancer treatment with a modulator of autophagy as otherwise described herein, the autophagy modulator is often an antagonist (down-regulator or inhibitor) of autophagy. In the case of cancer, the antagonist (inhibitor) of autophagy may be used alone or combined with an agonist of autophagy. In other instances, the modulator is an upregulator of autophagy.

The following compounds have been identified as additional autophagy modulators according to the present invention and can be used in the treatment of an autophagy mediated disease state or condition as otherwise described herein. These include interferon types I and II, especially interferon-alpha, interferon-beta, interferon interferon-gamma (IFN-gamma), pegylated interferon (PEG-IFN) type 1 or type 2 (especially including interferon alpha 2a and/or 2b), mixtures thereof, other cytokines and related compounds and mixtures thereof. In addition, Galectin proteins (1, 2, 3, 4, 7, 8, 9, 10, 12 and 13), preferably human Galectin proteins may also be included as autophagy modulators, especially Galectin-3, with Galectin-3 (Gal3) being especially preferred.

Agonists and/or inhibitors of Galectin-3 (Gal3), including a galactose containing sugar or other sugar compound (especially lactose, including N-linked and O-linked lactose such as N-acetyl lactosamine which acts as an agonist or an inhibitor such as a galactoside inhibitor or alternatively, a lactulose amine such as N-lactulose-octamethylenediamine (LDO); N,N-dilactulose-octamethylenediamine (D-LDO), and N,N-dilactulose-dodecamethylenediamine (D-LDD)), TD-1139 (GB0139), G3-C12, GR-MD-02, GM-CT-01, GCS-100, ipilimumab, a pectin (especially modified citrus pectin), or a taloside inhibitor may also be used.

In addition, the following sugars may also be used as inhibitors and/or agonists of Gal3). These sugars include, for example, monosaccharides, including β-galactoside sugars, such as galactose, including N- or O-linked (e.g., acetylated) galactosides and disaccharides, oligosaccharides and polysaccharides which contain at least one galactose sugar moiety. These include lactose, mannobiose, melibiose (which may have the glucose residue and/or the galactose residue optionally N-acetylated), melibiulose (which may have the galactose residue optionally N-acetylated), rutinose, (which may have the glucose residue optionally N-acetylated), rutinulose and xylobiose, among others, and trehalose, all of which can be N and O-linked. Oligosaccharides for use in the present invention as can include any sugar of three or more (up to about 100) individual sugar (saccharide) units as described above (i.e., any one or more saccharide units described above, in any order, especially including galactose units such as galactooligosaccharides and mannan-oligosaccharides ranging from three to about ten-fifteen sugar units in size. Sugars which are galactosides or contain galactose (galactose derivatives) are preferred for use in the present invention. These sugars may function as inhibitors or agonists of Gal3.

One or more of these above sugars may be combined with an agonist or antagonist of interferon, further optionally with a biguanide, a salicylate and a biguanide, berberine, ambroxol or a mixture thereof in order to upregulate autophagy and treat the autophagy diseases where upregulation is beneficial (e.g., inflammatory disease states and/or conditions including a microbial infection such as a Mycobacterium infection, among numerous others, an inflammatory disorder, a lysosomal storage disorder, an immune disorder, a neurodegenerative disorder, an autoimmune disease).

Useful Gal3 inhibitors include galactoside inhibitors or alternatively, a lactulose amine such as N-lactulose-octamethylenediamine (LDO); N,N-dilactulose-octamethylenediamine (D-LDO), and N,N-dilactulose-dodecamethylenediamine (D-LDD)), GR-MD-02, ipilimumab, a pectin, or a taloside inhibitor, among others may be used as an inhibitor of a galectin as described herein, especially galectin 3 (Gal3). These agents are particularly effective as anticancer agents with certain cancers especially when combined with Alix modulators (often an Alix inhibitor). These agents are also useful for the treatment of autoimmune diseases, including rheumatoid arthritis, antiphospholipid antibody syndrome, lupus, chronic urticaria or Sjogren's disease, as well as malaria.

Alternatively, one or more sugars described above may function as an inhibitor or agonist of Gal3 to be used in combination with an agonist or inhibitor of Alix (AIP1) (e.g. Alix itself, a peptide fragment agonist of Alix or a peptide antagonist of Alix and/or an anti-Alix antibody) for the treatment of certain cancers or an agonist or antagonist of TFRC. Agonists of Alix (AIP1) include calcium salts or calcium chelates including, for example, calcium carbonate, calcium citrate, calcium gluconate, calcium lactate, calcium phosphate, dicalcium malate, calcium hydroxy apatite, coral calcium or mixtures thereof. Antagonists of Alix (AIP1) include calcium chelators including, for example, ethylenediaminetetraacetic acid (EDTA) or a salt thereof (disodium, dipotassium, sodium potassium, sodium calcium or potassium calcium), ethyleneglycol-bis(2-aminoethylether)N,N,N′N′tetraacetic acid (egtazic acid or EGTA), EGTA acetoxymethyl ester (EGTA AM), 1,2-Bis(2-aminophenoxy)ethane-N,N,N′,N′-tetraacetic acid (BAPTA), BAPTA acetoxymethyl ester (BAPTA AM) and Poly(vinyl phosphonic acid-co-acrylic acid) (PVPA-coAA with mole ratios of VPA to acrylic acid ranging from 20:80 to 80:20) or a pharmaceutically acceptable salt thereof.

Agonists of TFRC include ferrous salts or ferrous chelates, for example, ferrous sulfate, ferrous fumarate, ferrous gluconate, ferrous succinate, ferrous sulfate, ferrous bis-glycinate, ferrous glycine sulfate, heme iron polypeptide, iron dextran, iron sucrose, iron carboxy maltose, iron maltoside and mixtures thereof. Antagonists of TFRC include iron chelators, for example, deferoxamine, deferasirox, Dp44mT, dexrazoxane, dexrazoxane HCl (ICRF-187), ciclopirox, pentetate calcium trisodium hydrate, 2,3-dihydroxybenzoic acid, VLX600, L-mimosine, N-NE3TA-NCS, CAB-NE3TA, DFT (2-(3′-hydroxypyrid-2′-yl)4-methyl-delta2-thiazoline-4(S)-carboxylic acid; desferrithiocin), 4-(OH)-DADFT, 4′-(HO)-DADMDFT ((S)-2-(2,4-dihydroxyphenyl)-4,5-dihydro-4-thiazolecarboxylic acid), BDU ((S,S)-1,11-bis[5-(4-carboxy-4,5-dihydrothiazol-2-yl)-2,4-dihydroxyphenyl]-4,8-dioxaundecane), ICL6770A (4-[3,5-bis-(hydroxyphenyl)-1,2,4-triazol-1-yl]-benzoic acid), DFP (3-Hydroxy-1,2-dimethyl-4(1H)-pyridone; Deferiprone), CP94 (Diethyl hydroxypyridinone), CP502 (1,6-dimethyl-3-hydroxy-4-(1H)-pyridinone-2-carboxy-(N-methyl)-amide hydrochloride), TREN-(Me-3,2-HOPO) (N,N′,N″-tris[(3-hydroxy-1-methyl-2-oxo-1,2-didehydropyrid-4-yl)carboxamidoethyl]amine), Pr-(Me-3,2-HOPO) (3-Hydroxy-1-methyl-2-oxo-1,2-dihydro-pyridine-4-carboxylic acid propylamide), Tachpyridine (N,N′,N,″-tris(2-pyridylmethyl)-cis,cis-1,3,5-triaminocyclohexane), PIH, SIH (Salicylaldehyde isonicotinoyl hydrazone), 311 (2-hydroxy-1-naphthylaldehyde isonicotinoyl hydrazone), 5-HP (5-hydroxypicolinaldehyde thiosemicarbazone), D-Exo 772SM, PCIH, INH, Deferitazole, EDTA, DTPA, Succimer, Trientine, BPS, PCTH, PCBH, PCBBH, PCAH, PCHH, FIH, Quercetin, or a pharmaceutically acceptable salt or mixture thereof.

The following compounds have been identified as additional autophagy modulators which may be used in combination with the above-identified autophagy modulator agents. These agents include, for example flubendazole, hexachlorophene, propidium iodide, bepridil, clomiphene citrate (Z, E), GBR 12909, propafenone, metixene, dipivefrin, fluvoxamine, dicyclomine, dimethisoquin, ticlopidine, memantine, bromhexine, ambroxol, norcyclobenzaprine, diperodon and nortriptyline, tetrachlorisophthalonitrile, phenylmercuric acetate and pharmaceutically acceptable salts thereof. It is noted that flubendazole, hexachlorophene, propidium iodide, bepridil, clomiphene citrate (Z, E), GBR 12909, propafenone, metixene, dipivefrin, fluvoxamine, dicyclomine, dimethisoquin, ticlopidine, memantine, bromhexine, norcyclobenzaprine, diperodon, nortriptyline, benzethonium, niclosamide, monensin, bromperidol, levobunolol, dehydroisoandosterone 3-acetate, sertraline, tamoxifen, reserpine, hexachlorophene, dipyridamole, harmaline, prazosin, lidoflazine, thiethylperazine, dextromethorphan, desipramine, mebendazole, canrenone, chlorprothixene, maprotiline, homochlorcyclizine, loperamide, nicardipine, dexfenfluramine, nilvadipine, dosulepin, biperiden, denatonium, etomidate, toremifene, tomoxetine, clorgyline, zotepine, beta-escin, tridihexethyl, ceftazidime, methoxy-6-harmalan, melengestrol, albendazole, rimantadine, chlorpromazine, pergolide, cloperastine, prednicarbate, haloperidol, clotrimazole, nitrofural, iopanoic acid, naftopidil, Methimazole, Trimeprazine, Ethoxyquin, Clocortolone, Doxycycline, Pirlindole mesylate, Doxazosin, Deptropine, Nocodazole, Scopolamine, Oxybenzone, Halcinonide, Oxybutynin, Miconazole, Clomipramine, Cyproheptadine, Doxepin, Dyclonine, Salbutamol, Flavoxate, Amoxapine, Fenofibrate, Pimethixene, and mixtures thereof.

The term “co-administration” or “combination therapy” is used to describe a therapy in which at least two active compounds in effective amounts are used to treat an autophagy mediated disease state or condition as otherwise described herein, either at the same time or within dosing or administration schedules defined further herein or ascertainable by those of ordinary skill in the art. Although the term co-administration preferably includes the administration of two active compounds to the patient at the same time, it is not necessary that the compounds be administered to the patient at the same time, although effective amounts of the individual compounds will be present in the patient at the same time. In addition, in certain embodiments, co-administration will refer to the fact that two compounds are administered at significantly different times, but the effects of the two compounds are present at the same time. Thus, the term co-administration includes an administration in which one active agent (especially an autophagy modulator) is administered at approximately the same time (contemporaneously), or from about one to several minutes to about 24 hours or more than the other bioactive agent coadministered with the autophagy modulator.

Additional bioactive agents may also be admininstered to treat autophagy mediated disease states and/or conditions pursuant to the present invention. Such bioactive agents be any bioactive agent, but is generally selected from an additional autophagy modulator compound or another agent such as a mTOR inhibitor such as Torin, pp242, rapamycin/serolimus (which also may function as an autophagy modulator), everolimus, temsirolomis, ridaforolimis, zotarolimis, 32-dexoy-rapamycin, among others including epigallocatechin gallate (EGCG), caffeine, curcumin or reseveratrol (which mTOR inhibitors find particular use as enhancers of autophagy using the compounds disclosed herein. In certain embodiments, an mTOR inhibitor selected from the group consisting of Torin, pp242, rapamycin/serolimus, everolimus, temsirolomis, ridaforolimis, zotarolimis, 32-dexoy-rapamycin, epigallocatechin gallate (EGCG), caffeine, curcumin or reseveratrol and mixtures thereof may be combined with at least one agent selected from the group consisting of digoxin, xylazine, hexetidine and sertindole, the combination of such agents being effective as autophagy modulators in combination.

The terms “cancer” and “neoplasia” are used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated.

As used herein, the terms malignant neoplasia and cancer are used synonymously to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, non-melanoma skin cancer (especially basal cell carcinoma or squamous cell carcinoma), acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. In certain aspects, the cancer which is treated is lung cancer, breast cancer, ovarian cancer and/or prostate cancer.

Neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms (cancer) are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, stomach and thyroid, leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma), mixed types of neoplasias, particularly carcinosarcoma and Hodgkin's disease, and tumors of mixed origin, such as Wilms' tumor and teratocarcinomas (Beers and Berkow (eds.), The Merck Manual of Diagnosis and Therapy, 17 sup.th ed. (Whitehouse Station, N.J.: Merck Research Laboratories, 1999) 973-74, 976, 986, 988, 991). All of these neoplasms may be treated using compounds according to the present invention.

Representative common cancers to be treated with compounds according to the present invention include, for example, prostate cancer, metastatic prostate cancer, stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. Because of the activity of the present compounds, the present invention has general applicability treating virtually any cancer in any tissue, thus the compounds, compositions and methods of the present invention are generally applicable to the treatment of cancer and in reducing the likelihood of development of cancer and/or the metastasis of an existing cancer.

In certain particular aspects of the present invention, the cancer which is treated is metastatic cancer, a recurrent cancer or a drug resistant cancer, especially including a drug resistant cancer. Separately, metastatic cancer may be found in virtually all tissues of a cancer patient in late stages of the disease, typically metastatic cancer is found in lymph system/nodes (lymphoma), in bones, in lungs, in bladder tissue, in kidney tissue, liver tissue and in virtually any tissue, including brain (brain cancer/tumor). Thus, the present invention is generally applicable and may be used to treat any cancer in any tissue, regardless of etiology.

The term “tumor” is used to describe a malignant or benign growth or tumefacent.

The term “additional anti-cancer compound”, “additional anti-cancer drug” or “additional anti-cancer agent” is used to describe any compound (including its derivatives) which may be used to treat cancer. The “additional anti-cancer compound”, “additional anti-cancer drug” or “additional anti-cancer agent” can be an anticancer agent which is distinguishable from a CIAE-inducing anticancer ingredient such as a taxane, vinca alkaloid and/or radiation sensitizing agent otherwise used as chemotherapy/cancer therapy agents herein. In many instances, the co-administration of another anti-cancer compound according to the present invention results in a synergistic anti-cancer effect. Exemplary anti-cancer compounds for co-administration with formulations according to the present invention include anti-metabolites agents which are broadly characterized as antimetabolites, inhibitors of topoisomerase I and II, alkylating agents and microtubule inhibitors (e.g., taxol), as well as tyrosine kinase inhibitors (e.g., surafenib), EGF kinase inhibitors (e.g., tarceva or erlotinib) and tyrosine kinase inhibitors or ABL kinase inhibitors (e.g. imatinib).

Anti-cancer compounds for co-administration include, for example, agent(s) which may be co-administered with compounds according to the present invention in the treatment of cancer. These agents include chemotherapeutic agents and include one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib, amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifostamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanioe, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinordbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxy tamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779, 450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidoniide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, ipilimumab, nivolomuab, pembrolizumab, dabrafenib, trametinib and vemurafenib among others.

Co-administration of one of the formulations of the invention comprising Gal3, Alix(AIP1) and/or a TFRC modulator with another anticancer agent will often result in a synergistic enhancement of the anticancer activity of the other anticancer agent, an unexpected result. One or more of the present formulations comprising a Gal3, Alix(AIP1) and/or a TFRC modulator optionally in combination with an additional autophagy modulator (autostatin) as described herein may also be co-administered with another bioactive agent (e.g., antiviral agent, antihyperproliferative disease agent, agents which treat chronic inflammatory disease, among others as otherwise described herein).

The term “antiviral agent” refers to an agent which may be used in combination with Gal3, Alix(AIP1) and/or a TFRC modulator optionally in combination with at least one additional autophagy modulator (autostatins) as otherwise described herein to treat viral infections, especially including HIV infections, HBV infections and/or HCV infections. Exemplary anti-HIV agents include, for example, nucleoside reverse transcriptase inhibitors (NRTI), non-nuclocoside reverse transcriptase inhibitors (NNRTI), protoase inhibitors, fusion inhibitors, among others, exemplary compounds of which may include, for example, 3TC (Lamivudine), AZT (Zidovudine), (-)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof, including anti-HIV compounds presently in clinical trials or in development. Exemplary anti-HBV agents include, for example, hepsera (adefovir dipivoxil), lamivudine, entecavir, telbivudine, tenofovir, emtricitabine, clevudine, valtoricitabine, amdoxovir, pradefovir, racivir, BAM 205, nitazoxanide, UT 231-B, Bay 41-4109, EHT899, zadaxin (thymosin alpha-1) and mixtures thereof. Anti-HCV agents include, for example, interferon, pegylated intergeron, ribavirin, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, ACH-1095, GSK625433, TG4040 (MVA-HCV), A-831, F351, NS5A, NS4B, ANA598, A-689, GNI-104, IDX102, ADX184, GL59728, GL60667, PSI-7851, TLR9 Agonist, PHX1766, SP-30 and mixtures thereof.

An “inflammation-associated metabolic disorder” is an autophagy mediated disorder which includes, but is not limited to, lung diseases, hyperglycemic disorders including diabetes and disorders resulting from insulin resistance, such as Type I and Type II diabetes, as well as severe insulin resistance, hyperinsulinemia, and dyslipidemia or a lipid-related metabolic disorder (e.g. hyperlipidemia (e.g., as expressed by obese subjects), elevated low-density lipoprotein (LDL), depressed high-density lipoprotein (HDL), and elevated triglycerides) and insulin-resistant diabetes, such as Mendenhall's Syndrome, Werner Syndrome, leprechaunism, and lipoatrophic diabetes, renal disorders, such as acute and chronic renal insufficiency, end-stage chronic renal failure, glomerulonephritis, interstitial nephritis, pyelonephritis, glomerulosclerosis, e.g., Kimmelstiel-Wilson in diabetic patients and kidney failure after kidney transplantation, obesity, GH-deficiency, GH resistance, Turner's syndrome, Laron's syndrome, short stature, increased fat mass-to-lean ratios, immunodeficiencies including decreased CD4⁺ T cell counts and decreased immune tolerance or chemotherapy-induced tissue damage, bone marrow transplantation, diseases or insufficiencies of cardiac structure or function such as heart dysfunctions and congestive heart failure, neuronal, neurological, or neuromuscular disorders, e.g., diseases of the central nervous system including Alzheimer's disease, or Parkinson's disease or multiple sclerosis, and diseases of the peripheral nervous system and musculature including peripheral neuropathy, muscular dystrophy, or myotonic dystrophy, and catabolic states, including those associated with wasting caused by any condition, including, e.g., mental health condition (e.g., anorexia nervosa), trauma or wounding or infection such as with a bacterium or human virus such as HIV, wounds, skin disorders, gut structure and function that need restoration, and so forth.

An “inflammation-associated metabolic disorder” is an autophagy mediated disorder which also includes a cancer and an “infectious disease” as defined herein, as well as disorders of bone or cartilage growth in children, including short stature, and in children and adults disorders of cartilage and bone in children and adults, including arthritis and osteoporosis. An “inflammation-associated metabolic disorder” includes a combination of two or more of the above disorders (e.g., osteoporosis that is a sequela of a catabolic state). Specific disorders of particular interest targeted for treatment herein are diabetes and obesity, heart dysfunctions, kidney disorders, neurological disorders, bone disorders, whole body growth disorders, and immunological disorders.

In one embodiment, “inflammation-associated metabolic disorder” includes: central obesity, dyslipidemia including particularly hypertriglyceridemia, low HDL cholesterol, small dense LDL particles and postpranial lipemia; glucose intolerance such as impaired fasting glucose, insulin resistance and hypertension, and diabetes. The term “diabetes” is used to describe diabetes mellitus type I or type II. The present invention relates to a method for improving renal function and symptoms, conditions and disease states which occur secondary to impaired renal function in patients or subjects with diabetes as otherwise described herein. It is noted that in diabetes mellitus type I and II, renal function is impaired from collagen deposits, and not from cysts in the other disease states treated by the present invention.

Mycobacterial infections often manifest as diseases such as tuberculosis. Human infections caused by Mycobacteria have been widespread since ancient times, and tuberculosis remains a leading cause of death today. Although the incidence of the disease declined, in parallel with advancing standards of living, since the mid-nineteenth century, mycobacterial diseases still constitute a leading cause of morbidity and mortality in countries with limited medical resources. Additionally, mycobacterial diseases can cause overwhelming, disseminated disease in immunocompromised patients. In spite of the efforts of numerous health organizations worldwide, the eradication of mycobacterial diseases has never been achieved, nor is eradication imminent. Nearly one third of the world's population is infected with Mycobacterium tuberculosis complex, commonly referred to as tuberculosis (TB), with approximately 8 million new cases, and two to three million deaths attributable to TB yearly. Tuberculosis (TB) is the cause of the largest number of human deaths attributable to a single etiologic agent (see Dye et al., J. Am. Med. Association, 282, 677-686, (1999); and 2000 WHO/OMS Press Release).

Mycobacteria other than M. tuberculosis are increasingly found in opportunistic infections that plague the AIDS patient. Organisms from the M. avium-intracellulare complex (MAC), especially serotypes four and eight, account for 68% of the mycobacterial isolates from AIDS patients. Enormous numbers of MAC are found (up to 10¹⁰ acid-fast bacilli per gram of tissue), and consequently, the prognosis for the infected AIDS patient is poor.

In many countries the only measure for TB control has been vaccination with M. bovis bacille Calmette-Guerin (BCG). The overall vaccine efficacy of BCG against TB, however, is about 50% with extreme variations ranging from 0% to 80% between different field trials. The widespread emergence of multiple drug-resistant M. tuberculosis strains is also a concern.

M. tuberculosis belongs to the group of intracellular bacteria that replicate within the phagosomal vacuoles of resting macrophages, thus protection against TB depends on T cell-mediated immunity. Several studies in mice and humans, however, have shown that Mycobacteria stimulate antigen-specific, major histocompatibility complex (MHC) class II- or class I-restricted CD4 and CD8 T cells, respectively. The important role of MHC class I-restricted CD8 T cells was convincingly demonstrated by the failure of β2-microglobulin) deficient mice to control experimental M. tuberculosis infection.

As used herein, the term “tuberculosis” comprises disease states usually associated with infections caused by Mycobacteria species comprising M. tuberculosis complex. The term “tuberculosis” is also associated with mycobacterial infections caused by Mycobacteria other than M. tuberculosis. Other mycobacterial species include M. avium-intracellulare, M. kansarii, M. fortuitum, M. chelonae, M. leprae, M. africanum, and M. microti, M. avium paratuberculosis, M. intracellulare, M. scrofulaceum, M. xenopi, M. marinum, M. ulcercms.

An “infectious disease” is an autophagy mediated disorder which includes but is limited to those caused by bacterial, mycological, parasitic, and viral agents. Examples of such infectious agents include the following: Staphylococcus, streptococcaceae, neisseriaaceae, cocci, enterobacteriaceae, pseudomonadaceae, vibrionaceae, Campylobacter, pasteurellaceae, Bordetella, Francisella, Brucella, legionellaceae, bacteroidaceae, gram-negative bacilli, Clostridium, Corynebacterium, Propionibacterium, gram-positive bacilli, anthrax, actinomyces, Nocardia, Mycobacterium, Treponema, Borrelia, Leptospira, Mycoplasma, Ureaplasma, Rickettsia, chlamydiae, systemic mycoses, opportunistic mycoses, protozoa, nematodes, trematodes, cestodes, adenoviruses, herpesviruses, poxviruses, papovaviruses, hepatitis viruses, orthomyxoviruses, paramyxoviruses, coronaviruses, picornaviruses, reoviruses, togaviruses, flaviviruses, bunyaviridae, rhabdoviruses, human immunodeficiency virus and retroviruses.

In certain embodiments, an “infectious disease” is selected from the group consisting of tuberculosis, leprosy, Crohn's Disease, aquired immunodeficiency syndrome, Lyme disease, cat-scratch disease, Rocky Mountain spotted fever and influenza or a viral infection selected from HIV (I and/or II), hepatitis B virus (HBV) or hepatitis C virus (HCV).

According to various embodiments, the combination of compositions and/or compounds according to the present invention may be used for treatment or prevention purposes in the form of a pharmaceutical composition. This pharmaceutical composition may comprise one or more of an active ingredient as described herein.

As indicated, the pharmaceutical composition may also comprise a pharmaceutically acceptable excipient, additive or inert carrier. The pharmaceutically acceptable excipient, additive or inert carrier may be in a form chosen from a solid, semi-solid, and liquid. The pharmaceutically acceptable excipient or additive may be chosen from a starch, crystalline cellulose, sodium starch glycolate, polyvinylpyrolidone, polyvinylpolypyrolidone, sodium acetate, magnesium stearate, sodium laurylsulfate, sucrose, gelatin, silicic acid, polyethylene glycol, water, alcohol, propylene glycol, vegetable oil, corn oil, peanut oil, olive oil, surfactants, lubricants, disintegrating agents, preservative agents, flavoring agents, pigments, and other conventional additives. The pharmaceutical composition may be formulated by admixing the active with a pharmaceutically acceptable excipient or additive.

The pharmaceutical composition may be in a form chosen from sterile isotonic aqueous solutions, pills, drops, pastes, cream, spray (including aerosols), capsules, tablets, sugar coating tablets, granules, suppositories, liquid, lotion, suspension, emulsion, ointment, gel, and the like. Administration route may be chosen from subcutaneous, intravenous, intestinal, parenteral, oral, buccal, nasal, intramuscular, transcutaneous, transdermal, intranasal, intraperitoneal, and topical. The pharmaceutical compositons may be immediate release, sustained/controlled release, or a combination of immediate release and sustained/controlled release depending upon the compound(s) to be delivered, the compound(s), if any, to be coadministered, as well as the disease state and/or condition to be treated with the pharmaceutical composition. A pharmaceutical composition may be formulated with differing compartments or layers in order to facilitate effective administration of any variety consistent with good pharmaceutical practice.

The subject or patient may be chosen from, for example, a human, a mammal such as domesticated animal, or other animal. The subject may have one or more of the disease states, conditions or symptoms associated with autophagy as otherwise described herein.

The compounds according to the present invention may be administered in an effective amount to treat or reduce the likelihood of an autophagy-mediated disease and/or condition as well one or more symptoms associated with the disease state or condition. One of ordinary skill in the art would be readily able to determine an effective amount of active ingredient by taking into consideration several variables including, but not limited to, the animal subject, age, sex, weight, site of the disease state or condition in the patient, previous medical history, other medications, etc.

For example, the dose of an active ingredient which is useful in the treatment of an autophagy mediated disease state, condition and/or symptom for a human patient is that which is an effective amount and may range from as little as 100 μg or even less to at least about 500 mg or more, which may be administered in a manner consistent with the delivery of the drug and the disease state or condition to be treated. In the case of oral administration, active is generally administered from one to four times or more daily. Transdermal patches or other topical administration may administer drugs continuously, one or more times a day or less frequently than daily, depending upon the absorptivity of the active and delivery to the patient's skin. Of course, in certain instances where parenteral administration represents a favorable treatment option, intramuscular administration or slow IV drip may be used to administer active. The amount of active ingredient which is administered to a human patient is an effective amount and preferably ranges from about 0.05 mg/kg to about 20 mg/kg, about 0.1 mg/kg to about 7.5 mg/kg, about 0.25 mg/kg to about 6 mg/kg, about 1.25 to about 5.7 mg/kg.

The dose of a compound according to the present invention may be administered at the first signs of the onset of an autophagy mediated disease state, condition or symptom. For example, the dose may be administered for the purpose of lung or heart function and/or treating or reducing the likelihood of any one or more of the disease states or conditions which become manifest during an inflammation-associated metabolic disorder or tuberculosis or associated disease states or conditions, including pain, high blood pressure, renal failure, or lung failure. The dose of active ingredient may be administered at the first sign of relevant symptoms prior to diagnosis, but in anticipation of the disease or disorder or in anticipation of decreased bodily function or any one or more of the other symptoms or secondary disease states or conditions associated with an autophagy mediated disorder to condition.

These and other aspects of the invention are described further in the following illustrative examples which are provided for illustration of the present invention and are not to be taken to limit the present invention in any way.

### EXAMPLES

### Experimental Models and Subject Details

### Mice

The generation of TRIM16fl/fl mouse founders directly in the C57BL/6 background was conducted by Cyagen. It involved mouse genomic fragment amplification from a BAC clone by using high fidelity Taq polymerase, and their sequential assembly into a targeting vector together with recombination sites and selection markers. The linearized vector was delivered to ES cells (C57BL/6) via electroporation, followed by drug selection, PCR screening and Southern blot detection. After confirming correctly targeted ES clones via Southern blotting, clones were selected for blastocyst microinjection, followed by chimera production. Founders were confirmed as germline-transmitted via crossbreeding with wild-type. TRIM16^{fl/fl} mice were bread with LysM-Cre C57BL/6 mice. Littermates for experiments were generated by breading TRIM16^{fl/fl}LysM-Cre⁺ mice with TRIM16^{fl/fl} mice to generate Cre⁺ and Cre⁻ littermates thus accounting for metagenomic considerations. Mice were 4-10 weeks old and included both sexes equally and near-equally represented in experimental groups. Genotyping was through Transnetyx services. All breeding procedures have been carried out following protocols approved by IACUC.

### Cell and Cell Line Models

Murine bone marrow derived macrophages (BMMs; primary cells) and authenticated cell lines were used for Murine tuberculosis infection analyses. Cell types, lines and culture conditions are described under Method Details.

### Murine tuberculosis Infection Model

Mycobacterium tuberculosis Erdman (Manzanillo et al., 2012) were cultured in Middlebrook 7H9 broth supplemented with 0.05% Tween 80, 0.2% glycerol, and 10% oleic acid, albumin, dextrose, and catalase (OADCBD Biosciences) at 37° C. and homogenized to generate single-cell suspension for macrophage infection studies. Mice were exposed to Mtb Erdman aerosols (intermediate dose of 1,372 CFU initial lung deposition as previously defined (Chauhan et al., 2016)) using a GlasCol apparatus for aerosol delivery, and survival monitored for 100 days post infection. All procedures have been carried out under ABSL3 conditions, following protocols approved by IACUC, and adherence to approved protocols was monitored by an independent direct observer.

### Method Details

### Antibodies and Reagents

Antibodies from Abcam were Galectin-3 (ab53082) (1:1000 for Western blot (WB); 1:200 for immunofluorescence (IF)), GFP (ab290) (1:1000 for WB), GFP (ab38689) (2/mL for immunoprecipitation (IP)), mCherry (ab183628) (1:1000 for WB; 1:200 for IF; 2 μg/mL for IP), VDAC1 (ab15895) (1:1000 for WB), CHMP4B (ab105767) (1:1000 for WB), CHMP4A (ab67058) (1:1000 for WB), GM130 (ab1299) (1:1000 for WB), P4HB (ab2792) (1:1000 for WB) and TSG101 (ab83) (1:1000 for WB). Antibodies from MBL International were LC3 (PM036) (1:500 for IF) and ATG16L1 (PM040) (1:400 for IF). Antibodies from BioLegend were Galectin-3 (#125402) (1:1000 for WB; 1:500 for IF) and ALIX (#634502) (1:1000 for WB; 1:400 for IF). Antibodies from Cell Signaling Technology were TFEB (#4240) (1:200 for IF), ATG13 (#13468) (1:200 for IF) and LAMP1 (#9091) (1:500 for IF). Other antibodies used in this study were from the following sources: FLAG M2 (F1804) (1:1000 for WB) from Sigma Aldrich; TRIM16 (A301-160A) (1:1000 for WB) from Bethyl; Myc (sc-40) (1:200 for WB), Galectin-8 (sc-28254) (1:200 for WB) and beta-Actin (C4) (1:1000 for WB), HRP-labeled anti-rabbit (1:2000 for WB) and anti-mouse (1:2000 for WB) secondary antibodies from Santa Cruz Biotechnology; LAMP2 (H4B4) (1:500 for IF) from DSHB of University of Iowa; Clean-Blot IP Detection Kit (HRP) (21232) (1:1000 for WB), Alexa Fluor 488, 568 (1:500 for IF), TFRC(#13-6800) (1:1000 for WB, 1:200 for IF) from ThermoFisher Scientific. DMEM, RPMI and EBSS medias from Life Technologies.

### Cells and Cell Lines

HEK293T, HeLa and huh7 cells were from ATCC. Bone marrow derived macrophages (BMMs) were isolated from femurs of TRIM16^{fl/fl}LysM-Cre mice and its Cre-negative littermates cultured in DMEM supplemented with mouse macrophage colony stimulating factor (mM-CSF, #5228, CST) HeLa Flp-In-Gal3^{TetON} were generated using constructs from Terje Johansen. Cell lines for LysoIP were generated using constructs obtained from David M. Sabatini (Whitehead Institute). CHO and Lec3.2.8 1 cells were from Pamela Stanley (Albert Einstein College of Medicine).

### Plasmids, siRNAs, and Transfection

Plasmids used in this study, such as ALIX and CHMP4A cloned into pDONR221 using BP cloning, and expression vectors were made utilizing LR cloning (Gateway, ThermoFisher) in appropriate pDEST vectors for immunoprecipitation assay.

Gal3 mutants were generated utilizing the QuikChange site-directed mutagenesis kit (Agilent) and confirmed by sequencing (Genewiz). siRNAs were from GE Dharmacon. Plasmid transfections were performed using the ProFection Mammalian Transfection System (Promega) or Amaxa nucleofection (Lonza). siRNAs were delivered into cells using either Lipofectamine RNAiMAX (ThermoFisher Scientific) or Amaxa nucleofection (Lonza).

### Generation of HeLa Flp-In-Gal3^{TetON} Cell Line

Transfect Hela Flp-In host cells with Gal3 reconstructed plasmid and the pOG44 expression plasmid at ration of 9:1. 24 h after transfection, wash the cells and add fresh medium to the cells 48 h after transfection, split the cells into fresh medium around 25% confluent. Incubate the cells at 37° C. for 2-3 h until they have attached to the culture dish. Then them medium was removed and added with fresh medium containing 100 μg/mL hygromyein. Feed the cells with selective medium every 3-4 days until single cell clone can be identified. Pick hygromycin-resistant clones and expand each clone to test. The tested clones incubated in the medium containing 1 μg/mL tetracycline overnight were determined by western blot for the expressing of Gal3.

### Generation of LysM-Cre TRIM16^{fl/fl} Mice

The generation of TRIM16fl/fl mouse founders directly in the C57BL/6 background was conducted by Cyagen. It involved mouse genomic fragment amplification from a BAC clone by using high fidelity Taq polymerase, and their sequential assembly into a targeting vector together with recombination sites and selection markers. The linearized vector was delivered to ES cells (C57BL/6) via electroporation, followed by drug selection, PCR screening and Southern blot detection. After confirming correctly targeted ES clones via Southern blotting, clones were selected for blastocyst microinjection, followed by chimera production. Founders were confirmed as germline-transmitted via crossbreeding with wild-type. TRIM16^{fl/fl} mice were bread with LysM-Cre C57BL/6 mice. Littermates for experiments were generated by breading TRIM16^{fl/fl}LysM-Cre⁺ mice with TRIM16^{fl/fl} mice to generate Cre⁺ and Cre⁻ littermates thus accounting for metagenomic considerations.

### LysoTracker Assay

Prepare fresh LysoTracker Staining Solution (2 μL LysoTracker in 1 mL medium). Add 10 μL LysoTracker Staining Solution to no treatment, 1 mM LLOMe treated or LLOMe washout cells in 96 wells for total 100 μL per well and incubate at 37° C. for 30 min protected from light. Rinse gently by 1×PBS and fix in 4% Paraformaldehyde for 2 min. Wash once by 1×PBS and blot with Hoechst 33342 for 2 min before detecting by high content microscopy.

### Magic Red Assay

Reconstitute Magic Red by adding DMSO and dilute Magic Red 1:10 by adding H₂O. Add 4 μL Magic Red to no treatment, 1 mM LLOMe treated or LLOMe washout cells in 96 wells for total 100 μL per well and incubate at 37° C. for 15 min and protected from light. Rinse gently by 1×PBS and fix in 4% Paraformaldehyde for 2 min. Wash once by 1×PBS and blot with Hoechst 33342 for 2 min before detecting by high content microscopy.

### LysoIP Assay

HEK293T cells were transfected with pLJC5-TMEM192-3xHA or pLJC5-TMEM192-2×FLAG constructs in combination with pCMV-VSV-G and psPAX2 packaging plasmids, 60 h after transfection, the supernatant containing lentiviruses was collected and centrifuged to remove cells and then frozen at −80° C. To establish LysoIP stably expressing cell lines, HEK293T, HeLa or HeLa Gal3^{KO} cells were plated in 10 cm dish in DMEM with 10% FBS and infected with 500 μL of virus-containing media overnight, then add puromycin for selection.

Cells in 15 cm plates with 90% confluency were used for each LysoIP. Cells with or without 1 mM LLOMe treatment were quickly rinsed twice with PBS and then scraped in 1 mL of KPBS (136 mM KCl, 10 mM KH₂PO₄, pH7.25 was adjusted with KOH) and centrifuged at 3000 rpm for 2 min at 4° C. Pelleted cells were resuspended in 950 μL KPBS and reserved 25 μL for further processing of the whole-cell lysate. The remaining cells were gently homogenized with 20 strokes of a 2 mL homogenizer. The homogenate was then centrifuged at 3000 rpm for 2 min at 40° C. and the supernatant was incubated with 100 μL of KPBS prewashed anti-HA magnetic beads (ThermoFisher) on a gentle rotator shaker for 3 min. Immunoprecipitants were then gently washed three times with KPBS and eluted with 2×Laemmli sample buffer (Bio-Rad) and subjected to immunoblot analysis.

### High Content Microscopy

Cells in 96 well plates were treated, followed by fixation in 4% paraformaldehyde for 5 min. Cells were then permeabilized with 0.1% saponin in 3% Bovine serum albumin (BSA) for 30 min followed by incubation with primary antibodies for 2 h and secondary antibodies for 1 h. High content microscopy with automated image acquisition and quantification was carried out using a Cellomics HCS scanner and iDEV software (ThermoFisher Scientific). Automated epifluorescence image collection was performed for a minimum of 500 cells per well. Epifluorescence images were machine analyzed using preset scanning parameters and object mask definitions Hoechst 33342 staining was used for autofocus and to automatically define cellular outlines based on background staining of the cytoplasm. Primary objects were cells, and regions of interest (ROI) or targets were algorithm-defined by shape/segmentation, maximum/minimum average intensity, total area and total intensity, etc., to automatically identify puncta or other profiles within valid primary objects. Nuclei were defined as a region of interest for TFEB translocation. All data collection, processing (object, ROI, and target mask assignments) and analyses were computer driven independently of human operators.

### Super-Resolution Imaging and Analysis

Super-resolution imaging and analysis were done as described previously (Kumar et al., 2018; Pallikkuth et al., 2018). HeLa cells transfected with GFP-Gal3 were plated on 25 mm round #1.5 coverslips (Warner Instruments) coated with Poly-L-Lysine solution (Sigma-Aldrich) and allowed to adhere overnight. After two steps fixation (first step (0.6% PFA, 0.1% Glyoxal solution (GA), 0.25% Triton X-100) for 60 sec; second step (4% PFA, 0.2% GA) for 3 h), cells were washed by 1×PBS twice, and incubated with 0.1% NaBH4 for 5 min. After washed by 1×PBS twice, cells were incubated with 10 mM Tris for 5 min, then block cells with 5% BSA containing 0.05% Triton X-100 for 15 min. After washed by 1×PBS once, cells were incubated with anti-rabbit-GFP antibody for 2 h and washed with 1×PBS three times followed by labeling with Alexa Fluor 647 (A21245, Invitrogen). The coverslip was mounted on an Attofluor cell chamber (A-7816, ThermoFisher Scientific) with 1.1 mL of the imaging buffer. Imaging buffer consisted of an enzymatic oxygen-scavenging system and primary thiol: 50 mM Tris, 10 mM NaCl, 10% (wt/vol) glucose, 168.8 U/mL glucose oxidase (G2133, Sigma-Aldrich), 1,404 U/mL catalase (C9332, Sigma-Aldrich), and 20 mM 2-aminoethanethiol, pH8. The chamber was sealed by placing an additional coverslip over the chamber, and the oxygen-scavenging reaction was allowed to proceed for 20 min at room temperature before the imaging started.

Imaging was performed using a custom-built microscope controlled by custom-written software (github.com/LidkeLab/matlab-instrument-control) in MATLAB (MathWorks Inc.). The samples were loaded on an xyz piezo stage (Mad City Labs, Nano-LPS100) mounted on a manual x-y translator. Images were recorded on an iXon 897 electron-multiplying charge coupled (EMCCD) camera (Andor Technologies, South Windsor, Conn.). The EMCCD gain was set to 100, and 256×256 pixel frames were collected with a pixel resolution of 0.1078 □m. A 642-nm laser (collimated from a laser diode, HL6366DG, Thorlabs) was used for sample excitation. The laser was coupled into a multi-mode fiber (P1-488PM-FC-2, Thorlabs) and focused onto the back focal plane of the objective lens (UAPON 150XOTIRF, Olympus America Inc.). Sample excitation was done through a quad-band dichroic and filter set (LF405/4S8/561/635-A; Semrock, Rochester, N.Y.). Fluorescence emission path included a band-pass filter (685/45, Brightline) and a quadband optical filter (Photometrics, QV2-SQ) with 4 filter sets (600/37, 525/45, 685/40, 445/45, Brightline).

When imaging the first label (GFP-Gal3), for each target cell a brightfield reference image was saved in addition to the x-y stage position coordinates. The 642-nm laser was used at ˜1 kW/cm2 to take 20 sets of 2,000 frames (a total of 40,000) at 60 Hz. After imaging all target cells, imaging buffer was replaced with 1×PBS, the residual fluorescence was photobleached, quenched with NaBH4 and washed thrice with 1×PBS. Before the second round of imaging, cells were blocked for 30 min, labeled with anti-ALIX antibody (#634502, BioLegend) for 1 h, washed with 1×PBS three times and relabeled using an Alexa Fluor 647 conjugated anti-mouse antibody at 10 μg/mL for 1 h. Before the second round of imaging, each target cell was located and realigned using the saved brightfield reference image as described in (Valley et al., 2015).

Data were analyzed via a 2D localization algorithm based on maximum likelihood estimation (Smith et al., 2010). The localized emitters were filtered through thresholds of maximum background photon counts of 200, minimum photon counts per frame per emitter of 250, and a data model hypothesis test (Huang et al., 2011) with a minimum p-value of 0.01. The accepted emitters were used to reconstruct the super-resolution image. Each emitter was represented by a 2D Gaussian function with σₓ and σ_{y} equal to the localization precisions, which were calculated from the Cramér-Rao Lower Bound (CRLB). Clustering analysis was performed with MATLAB code using clustering tools (http://stmc.health.unm.edu/tools-and-data/). Several regions of interest (ROIs) were selected from the image. Clustering was then performed separately for each label in each ROI using the density-based DBSCAN algorithm choosing a maximal nearest neighbor distance of 40 nm and requiring clusters to contain at least 5 observations. In all cases, most observations for each label in each ROI formed a single cluster. Cluster boundaries were produced via the MATLAB “boundary” function, from which inter-label cluster distances were computed.

### Time-Lapse Imaging of Cultured Cells

Wild type or Gal3^{KO} HeLa cell expressing mCherry-ALIX and GFP-LAMP1, were treated with 15 μM BAPTA-AM for 1 h and then incubated with 1 mM LLOMe for live-cell fluorescence image which was performed using an inverted microscope (confocal TCS SP5, Leica, LAS AF version 2.6.0), a 63× PlanAPO oil-immersion objective lens (NA 1.4). Two-color time-lapse images were acquired at 340 ms intervals and z-stacks collapsed into 2D projections to generate movies.

### Co-Immunoprecipitation Assay

Cells transfected with 8-10 μg of plasmids were lysed in NP-40 buffer (ThermoFisher Scientific) supplemented with protease inhibitor cocktail (Roche, 11697498001) and 1 mM PMSF (Sigma, 93482) for 30 min on ice. Supernatants were incubated with (2-3 μg) antibodies overnight at 4° C. The immune complexes were captured with Dynabeads (ThermoFisher Scientific), followed by three times washing with 1×PBS. Proteins bound to Dynabeads were eluted with 2×Laemmli sample buffer (Bio-Rad) and subjected to immunoblot analysis.

### APEX2-Labeling and Streptavidin Enrichment for Immunoblotting Analyses

HEK293T cells transfected pJJiaDEST-APEX2-Gal3 were incubated with 100 μM GPN (Cayman Chemicals) or 1 mM LLOMe in full medium for 1 h (confluence of cells remained at 70-80%). Cells were next incubated in 500 μM biotin-phenol (AdipoGen) in full medium for the last 45 min of GPN or LLOMe incubation. A 1 min pulse with 1 mM H₂O₂ at room temperature was stopped with quenching buffer (10 mM sodium ascorbate, 10 mM sodium azide and 5 mM Trolox in Dulbecco's Phosphate Buffered Saline (DPBS)). All samples were washed twice with quenching buffer, and twice with DPBS.

For LC/MS/MS analysis, cell pellets were lysed in 500 μL ice-cold lysis buffer (6 M urea, 0.3 M Nacl, 1 mM EDTA, 1 mM EGTA, 10 mM sodium ascorbate, 10 mM sodium azide, 5 mM Trolox, 1% glycerol and 25 mm Tris/HCl, PH 7.5) for 30 min by gentle pipetting. Lysates were clarified by centrifugation and protein concentrations determined as above. Streptavidin-coated magnetic beads (Pierce) were washed with lysis buffer. 3 mg of each sample was mixed with 100 μL of streptavidin bead. The suspensions were gently rotated at 4° C. for overnight to bind biotinylated proteins. The flowthrough after enrichment was removed and the beads were washed in sequence with 1 mL IP buffer (150 mM NaCl, 10 mM Tris-HCl pH8.0, 1 mM EDTA, 1 mM EGTA, 1% Triton X-100) twice; 1 mL 1M KCl; 1 mL of 50 mM Na₂CO₃; 1 mL 2M Urea in 20 mM Tris HCl pH8; 1 mL IP buffer Biotinylated proteins were eluted, 10% of the sample processed for Western Blot and 90% of the sample processed for mass spectrometry.

Protein samples on magnetic beads were washed four times with 200 ul of 50 mM Triethyl ammonium bicarbonate (TEAB) with a 20 minutes shake time at 4° C. in between each wash. Roughly 2.5 μg of trypsin was added to the bead and TEAB mixture and the samples were digested over night at 800 rpm shake speed. After overnight digestion the supernatant was removed, and the beads were washed once with enough 50 mM ammonium bicarbonate to cover. After 20 minutes at a gentle shake the wash is removed and combined with the initial supernatant. The peptide extracts are reduced in volume by vacuum centrifugation and a small portion of the extract is used for fluorometric peptide quantification (Thermo scientific Pierce). One microgram of sample based on the fluorometric peptide assay was loaded for each LC/MS analysis.

### LC/MS/MS DDA

Digested peptides were analyzed by LC/MS/MS on a Thermo Scientific Q Exactive Plus Orbitrap Mass spectrometer in conjunction Proxeon Easy-nLC II HPLC (Thermo Scientific) and Proxeon nanospray source. The digested peptides were loaded a 100-micron×25 mm Magic C18 100 Å 5 U reverse phase trap where they were desalted online before being separated using a 75-micron×150 mm Magic C18 200Å 3 U reverse phase column. Peptides were eluted using a 140 min gradient with a flow rate of 300 nL/min. An MS survey scan was obtained for the m/z range 350-1600, MS/MS spectra were acquired using a top 15 method, where the top 15 ions in the MS spectra were subjected to HCD (High Energy Collisional Dissociation). An isolation mass window of 1.6 m/z was for the precursor ion selection, and normalized collision energy of 27% was used for fragmentation. A fifteen-second duration was used for the dynamic exclusion.

### LC/MS/MS DIA

Peptides were separated on an Easy-spray 100 μm×25 cm C18 column using a Dionex Ultimate 3000 nUPLC. Solvent A=0.1% formic acid, Solvent B=100% Acetonitrile 0.1% formic acid. Gradient conditions=2% B to 50% B over 60 minutes, followed by a 50%-99% B in 6 minutes and then held for 3 minutes than 99% B to 2% B in 2 minutes. Total Run time=90 minutes. Thermo Scientific Fusion Lumos mass spectrometer running in Data independent Analysis mode. Two gas phases fractionated (GFP) injections were made per sample using sequential 4 Da isolation widows. GFP1=m/z 362-758, GFP2=m/z 758-1158. Tandem mass spectra were acquired using a collision energy of 30, resolution of 30K, maximum inject time of 54 ms and a AGC target of 50K.

### DDA Quantification and Statistical Analysis

Mass spectrometry data processing and analysis were done as described previously (Jia et al., 2018). Tandem mass spectra were extracted by Proteome Discoverer version 2.2. Charge state deconvolution and deisotoping were not performed. All MS/MS samples were analyzed using Sequest-HT (XCorr Only) (Thermo Fisher Scientific, San Jose, Calif., USA; in Proteome Discoverer 2.2.0.388). Sequest (XCorr Only) was set up to search the gpm common laboratory contaminants and the Uniprot human proteome 3AUP000005640 with isoforms (August 2017, 93299 entries) assuming the digestion enzyme trypsin. Sequest (XCorr Only) was searched with a fragment ion mass tolerance of 0.020 Da and a parent ion tolerance of 10.0 PPM. Carbamidomethyl of cysteine was specified in Sequest (XCorr Only) as a fixed modification. Deamidated of asparagine, oxidation of methionine and acetyl of the n-terminus were specified in Sequest (XCorr Only) as variable modifications. Precursor intensity was determined using Proteome Discoverer 2.2 using the Minora Feature detector with the default options.

Scaffold (version Scaffold_4.8.2, Proteome Software Inc., Portland, Oreg.) was used to validate MS/MS based peptide and protein identifications. Peptide identifications were accepted if they could be established at greater than 93.0% probability to achieve an FDR less than 0.1% by the Scaffold Local FDR algorithm. Protein identifications were accepted if they could be established at greater than 99.0% probability and contained at least two identified peptides. This filtering resulted in a decoy false discovery rate of 0.08% on the spectra level and 0.7% on the protein level. Protein probabilities were assigned by the Protein Prophet algorithm. Proteins that contained similar peptides and could not be differentiated based on MS/MS analysis alone were grouped to satisfy the principles of parsimony. Proteins sharing significant peptide evidence were grouped into clusters. Raw data and ScaffoldDIA results are available from the MassIVE proteomics repository (MSV000083998) and Proteome Exchange PXD014304. Reviewer password for Proteome Exchange=Galectin3.

### DIA Quantification and Statistical Analysis

DIA data was analyzed using Scaffold DIA (1.3.1). Raw data files were convened to mzML format using ProteoWizard (3.0.11748). Analytic samples were aligned based on retention times and individually searched against Pan human library http://www.swathatlas.org/ with a peptide mass tolerance of 10.0 ppm and a fragment mass tolerance of 10.0 ppm. Variable modifications considered were: Modification on M M and Modification on C C. The digestion enzyme was assumed to be Trypsin with a maximum of 1 missed cleavage site(s) allowed. Only peptides with charges in the range <2 . . . 3> and length in the range <6 . . . 30> were considered. Peptides identified in each sample were filtered by Percolator (3.01 nightly-13-655e4c7-dirty) to achieve a maximum FDR of 0.01. Individual search results were combined and peptide identifications were assigned posterior error probabilities and filtered to an FDR threshold of 0.01 by Percolator (3.01.nightly-13-655e4c7-dirty).

Peptide quantification was performed by Encyclopedia (0.8.1). For each peptide, the 5 highest quality fragment ions were selected for quantitation. Proteins that contained similar peptides and could not be differentiated based on MS/MS analysis were grouped to satisfy the principles of parsimony. Proteins with a minimum of 2 identified peptides were thresholded to achieve a protein FDR threshold of 1.0%. Raw data and ScaffoldDIA results are available from the MassIVE proteomics repository (MSV000083998) and Proteome Exchange PXD014304. Reviewer password for Proteome Exchange=Galectin3.

### Data and Code Availability

Original microscopy and Western blots of this study have been deposited at Mendeley data http://dx.doi.org/10.17632/v52k86mp58.1; Raw MS DIA/DDA data have been deposited at the MassIVE proteomics repository (MSV000083998) and Proteome Exchange PXD014304.

### Quantification and Statistical Analysis

Data in this study are presented as means±SEM (n≥3). Data were analyzed with either analysis of variance (ANOVA) with Tukey's HSD post-hoc test, or a two-tailed Student's t test. For HCM, n≥3 includes in each independent experiment: 500 valid primary objects/cells per well, from ≥5 wells per plate per sample. Animal survival data were analyzed by log-rank (Mantel-Cox) method. Statistical significance was defined as: † (not significant) p≥0.05 and *p≤0.05, **p<0.01.

### REFERENCES

Abu-Remaileh, M., Wyant, G. A., Kim, C., Laqtom, N. N., Abbasi, M., Chan, S. H., Freinkman, E., and Sabatini, D. M. (2017). Lysosomal metabolomics reveals V-ATPase- and mTOR-dependent regulation of amino acid efflux from lysosomes. Science 358, 807-813.
Aits, S., Kricker, J., Liu, B., Ellegaard, A. M., Hamalisto, S., Tvingsholm, S., Corcelle-Termeau, E., Hogh, S., Farkas, T., Holm Jonassen, A., et al. (2015). Sensitive detection of lysosomal membrane permeabilization by lysosomal galectin puncta assay. Autophagy 11, 1408-1424.
Baietti, M. F., Zhang, Z., Mortier, E., Melchior, A., Degeest, G., Geeraerts, A., Ivarsson, Y., Depoortere, F., Coomans, C., Vermeiren, E., et al. (2012). Syndecan-syntenin-ALIX regulates the biogenesis of exosomes. Nat Cell Biol 14, 677-685.
Bakula, D., Muller, A. J., Zuleger, T., Takacs, Z., Franz-Wachtel, M., Thost, A. K., Brigger, D., Tschan, M. P., Frickey, T., Robenek, H., et al. (2017). WIPI3 and WIPI4 beta-propellers are scaffolds for LKB1-AMPK-TSC signalling circuits in the control of autophagy. Nature communications 8, 15637.
Baskaran. S., Carlson, L. A., Stjepanovic, G., Young, L. N., Kim do, J., Grob, P., Stanley, R. E., Nogales, E., and Hurley, J. H. (2014). Architecture and dynamics of the autophagic phosphatidylinositol 3-kinase complex. Elife 3.
Berg, T. O., Stromhaug, E., Lovdal, T., Seglen, O., and Berg, T. (1994). Use of glycyl-L-phenylalanine 2-naphthylamide, a lysosome-disrupting cathepsin C substrate, to distinguish between lysosomes and prelysosomal endocytic vacuoles. Biochem J 300 (Pt 1), 229-236.
Birgisdottir, A. B., Lamark, T., and Johansen, T. (2013). The LIR motif—crucial for selective autophagy. Journal of cell science 126, 3237-3247.
Bjorkoy, G., Lamark, T., Brech, A., Outzen, H., Perander, M., Overvatn, A., Stenmark, H., and Johansen, T. (2005). p62/SQSTM1 forms protein aggregates degraded by autophagy and has a protective effect on huntingtin-induced cell death. J Cell Biol 171, 603-614.
Carlton, J. G., and Martin-Serrano, J. (2007). Parallels between cytokinesis and retroviral budding: a role for the ESCRT machinery. Science 316, 1908-1912.
Castillo, E. F., Dekonenko, A., Arko-Mensah, J., Mandell, M. A., Dupont, N., Jiang, S., Delgado-Vargas, M., Timmins, G. S., Bhattacharya, D., Yang, H., et al. (2012). Autophagy protects against active tuberculosis by suppressing bacterial burden and inflammation. Proceedings of the National Academy of Sciences of the United States of America 109, E3168-3176.
Chang, C., Young, L. N., Morris, K. L., von Bulow, S., Schoneberg, J., Yamamoto-Imoto, H., Oe, Y., Yamamoto, K., Nakamura, S., Stjepanovic, G., et al. (2019). Bidirectional Control of Autophagy by BECN1 BARA Domain Dynamics. Mol Cell 73, 339-353 e336.
Chauhan, S., Kumar, S., Jain, A., Ponpuak, M., Mudd, M. H., Kimura, T., Choi, S. W., Peters, R., Mandell, M., Bruun, J. A., et al. (2016). TRIMs and Galectins Globally Cooperate and TRIM16 and Galectin-3 Co-direct Autophagy in Endomembrane Damage Homeostasis. Dev Cell 39, 13-27.
Choudhuri, K., Llodra, J., Roth, E. W., Tsai, J., Gordo, S., Wucherpfennig, K. W., Kam, L. C., Stokes, D. L., and Dustin, M. L. (2014). Polarized release of T-cell-receptor-enriched microvesicles at the immunological synapse. Nature 507, 118-123.
Christ, L., Raiborg, C., Wenzel, E. M., Campsteijn, C., and Stenmark, H. (2017). Cellular Functions and Molecular Mechanisms of the ESCRT Membrane-Scission Machinery Trends Biochem Sci 42, 42-56.
Dauner, K., Eid, W., Raghupathy, R., Presley, J. F., and Zha, X. (2017). mTOR complex 1 activity is required to maintain the canonical endocytic recycling pathway against lysosomal delivery. The Journal of biological chemistry 292, 5737-5747.
Dautry-Varsat, A., Ciechanover, A., and Lodish, H. F. (1983). pH and the recycling of transferrin during receptor-mediated endocytosis. Proc Natl Acad Sci USA 80, 2258-2262.
Denais, C. M., Gilbert, R. M., Isermann, P., McGregor, A. L., te Lindert, M., Weigelin, B., Davidson, P. M., Friedl, P., Wolf, K., and Lammerding, J. (2016). Nuclear envelope rupture and repair during cancer cell migration Science 352, 353-358.
Di Lella, S., Sundblad, V., Cerliani, J. P., Guardia, C. M., Estrin, D. A., Vasta, G. R., and Rabinovich, G. A. (2011). When galectins recognize glycans: from biochemistry to physiology and back again. Biochemistry 50, 7842-7857.
Do, S. I., Enns, C., and Cummings, R. D. (1990). Human transferrin receptor contains O-linked oligosaccharides. The Journal of biological chemistry 265, 114-125.
Dooley, H. C., Razi, M., Polson, H. E., Girardin, S. E., Wilson, M. I., and Tooze, S. A. (2014). WIPI2 Links LC3 Conjugation with PI3P, Autophagosome Formation, and Pathogen Clearance by Recruiting Atg12-5-l6L1. Mol Cell 55, 238-252.
Dupont, N., Lacas-Gervais, S., Bertout, J., Paz, I., Freche, B., Van Nhieu, G. T., van der Goot, F. G., Sansonetti, P. J., and Lafont, F. (2009). Shigella phagocytic vacuolar membrane remnants participate in the cellular response to pathogen invasion and are regulated by autophagy. Cell Host Microbe 6, 137-149.
Dussupt, V., Javid, M. P., Abou-Jaoude, G., Jadwin, J. A., de La Cruz, J., Nagashima, K., and Bouamr, F. (2009). The nucleocapsid region of HIV-1 Gag cooperates with the PTAP and LYPXnL late domains to recruit the cellular machinery necessary for viral budding PLoS Pathog 5, e1000339.
Feeley, E. M., Pilla-Moffett, D. M., Zwack, E. E., Piro, A. S., Finethy, R., Kolb, J. P., Martinez, J., Brodsky, I. E., and Coers, J. (2017). Galectin-3 directs antimicrobial guanylate binding proteins to vacuoles furnished with bacterial secretion systems. Proc Natl Acad Sci USA 114, E1698-E1706.
Fisher, R. D., Chung, H. Y., Zhai, Q., Robinson, H., Sundquist, W. I., and Hill, C. P. (2007). Structural and biochemical studies of ALIX/AIP1 and its role in retrovirus budding. Cell 128, 841-852.
Fujii, K., Munshi, U. M., Ablan, S. D., Demirov, D. G., Soheilian, F., Nagashima, K., Stephen, A. G., Fisher, R. J., and Freed, E. O. (2009). Functional role of Alix in HIV-1 replication. Virology 391, 284-292.
Fujita, N., Morita, E., Itoh, T., Tanaka, A., Nakaoka, M., Osada, Y., Umemoto, T., Saitoh, T., Nakatogawa, H., Kobayashi, S., et al. (2013). Recruitment of the autophagic machinery to endosomes during infection is mediated by ubiquitin. J Cell Biol 203, 115-128.
Gammoh, N., Florey, O., Overholtzer, M., and Jiang. X. (2013). Interaction between FIP200 and ATG16L1 distinguishes ULK1 complex-dependent and -independent autophagy. Nat Struct Mol Biol 20, 144-149.
Ganley, I. G., Lam du, H., Wang, J., Ding, X., Chen, S., and Jiang, X. (2009). ULK1.ATG13.FIP200 complex mediates mTOR signaling and is essential for autophagy. The Journal of biological chemistry 284, 12297-12305.
Garcia, D., and Shaw, R. J. (2017). AMPK: Mechanisms of Cellular Energy Sensing and Restoration of Metabolic Balance Mol Cell 66, 789-800.
Garrus, J. E., von Schwedler, U. K., Pornillos, O. W., Morham, S. G., Zavitz, K. H., Wang, H. E., Wettstein, D. A., Stray, K. M., Cote, M., Rich, R. L., et al. (2001). Tsg101 and the vacuolar protein sorting pathway are essential for HIV-1 budding. Cell 107, 55-65.
Glenn, K. A., Nelson, R. F., Wen, H. M., Mallinger, A. J., and Paulson, H. L. (2008). Diversity in tissue expression, substrate binding, and SCF complex formation for a lectin family of ubiquitin ligases. The Journal of biological chemistry 283, 12717-12729.
Gong, Y. N., Guy, C., Olauson, H., Becker, J. U., Yang, M., Fitzgerald. P., Linkermann, A., and Green, D. R. (2017). ESCRT-III Acts Downstream of MLKL to Regulate Necroptotic Cell Death and Its Consequences. Cell 169, 286-300 e216.
Gutierrez, M. G., Master, S. S., Singh, S. B., Taylor, G. A., Colombo, M. I., and Deretic, V. (2004). Autophagy is a defense mechanism inhibiting BCG and Mycobacterium tuberculosis survival in infected macrophages. Cell 119, 753-766.
He, C., and Levine, B. (2010). The Beclin 1 interactome. Current opinion in cell biology 22, 140-149.
Henne, W. M., Buchkovich, N. J., and Emr, S. D. (2011). The ESCRT pathway Dev Cell 21, 77-91.
Hornung, V., Bauernfeind, F., Halle, A., Samstad, E. O., Kono, H., Rock, K. L., Fitzgerald, K. A., and Latz, E. (2008). Silica crystals and aluminum salts activate the NALP3 inflammasome through phagosomal destabilization. Nat Immunol 9, 847-856.
Hosokawa, N., Hara, T., Kaizuka, T., Kishi, C., Takamura, A., Miura, Y., Iemura, S., Natsume, T., Takehana, K., Yamada, N., et al. (2009). Nutrient-dependent mTORC1association with the ULK1-Atg13-FIP200 complex required for autophagy. Mol Biol Cell 20, 1981-1991.
Houzelstein, D., Goncalves, I. R., Fadden, A. J., Sidhu, S. S., Cooper, D. N., Drickamer, K., Leffler, H., and Poirier, F. (2004). Phylogenetic analysis of the vertebrate galectin family. Mol Biol Evol 21, 1177-1187.
Huang, F., Schwartz, S. L., Byars, J. M., and Lidke, K. A. (2011). Simultaneous multiple-emitter fitting for single molecule super-resolution imaging. Biomed Opt Express 2, 1377-1393.
Hurley, J. H. (2015). ESCRTs are everywhere. EMBO J 34, 2398-2407.
Hurley, J. H., and Hanson, P. I. (2010). Membrane budding and scission by the ESCRT machinery: it's all in the neck. Nat Rev Mol Cell Biol 11, 556-566.
Jao, C. C., Ragusa, M. J., Stanley, R. E., and Hurley, J. H. (2013). A HORMA domain in Atg13 mediates PI 3-kinase recruitment in autophagy. Proc Natl Acad Sci USA 110, 5486-5491.
Jia, J., Abudu, Y. P., Claude-Taupin, A., Gu, Y., Kumar, S., Choi, S. W., Peters, R., Mudd, M. H., Allers, L., Salemi, M., et al. (2018). Galectins Control mTOR in Response to Endomembrane Damage. Mol Cell 70, 120-135 e128.
Jimenez, A. J., Maiuri, P., Lafaurie-Janvore, J., Divoux, S., Piel, M., and Perez, F. (2014). ESCRT machinery is required for plasma membrane repair. Science 343, 1247136.
Johannes, L., Jacob, R., and Leffler, H. (2018). Galectins at a glance. J Cell Sci 131.
Jung. C. H., Jun, C. B., Ro, S. H., Kim, Y. M., Otto. N. M., Cao, J., Kundu, M., and Kim, D. H. (2009). ULK-Atg13-FIP200 complexes mediate mTOR signaling to the autophagy machinery. Mol Biol Cell 20, 1992-2003.
Kabeya, Y., Mizushima, N., Ueno, T., Yamamoto, A., Kirisako, T., Noda, T., Kominami, E., Ohsumi, Y., and Yoshimori, T. (2000). LC3, a mammalian homologue of yeast Apg8p, is localized in autophagosome membranes after processing. Embo J 19, 5720-5728.
Karanasios, E., Stapleton, E., Manifava, M., Kaizuka, T., Mizushima, N., Walker, S. A., and Ktistakis, N. T. (2013). Dynamic association of the ULK1 complex with omegasomes during autophagy induction. J Cell Sci 126, 5224-5238.
Karanasios, E., Walker, S. A., Okkenhaug, H., Manifava, M., Hummel, E., Zimmermann, H., Ahmed, Q., Domart, M. C., Collinson, L., and Ktistakis, N. T. (2016). Autophagy initiation by ULK complex assembly on ER tubulovesicular regions marked by ATG9 vesicles. Nature communications 7, 12420.
Katzmann, D. J., Odorizzi, G., and Emr, S. D. (2002). Receptor downregulation and multivesicular-body sorting. Nat Rev Mol Cell Biol 3, 893-905.
Kim, J., Kim, Y. C., Fang, C., Russell, R. C., Kim, J. H., Fan, W., Liu, R., Zhong. Q., and Guan, K. L. (2013). Differential regulation of distinct Vps34 complexes by AMPK in nutrient stress and autophagy. Cell 152, 290-303.
Kim, J., Kundu, M., Viollet, B., and Guan, K. L. (2011). AMPK and mTOR regulate autophagy through direct phosphorylation of Ulk1. Nat Cell Biol 13, 132-141.
Kimura, T., Jia, J., Kumar, S., Choi, S. W., Gu, Y., Mudd, M., Dupont, N., Jiang, S., Peters, R., Farzam, F., et al. (2017). Dedicated SNAREs and specialized TRIM cargo receptors mediate secretory autophagy. EMBO J 36, 42-60.
Kimura, T., Mandell, M., and Deretic, V. (2016). Precision autophagy directed by receptor regulators—emerging examples within the TRIM family. J Cell Sci 129, 881-891.
Knorr, R. L., Lipowsky, R., and Dimova, R. (2015). Autophagosome closure requires membrane scission. Autophagy 11, 2134-2137.
Kumar, S., Jain, A., Farzam, F., Jia, J., Gu, Y., Choi, S. W., Mudd, M. H., Claude-Taupin, A., Wester, M. J., Lidke, K. A., et al. (2018). Mechanism of Stx17 recruitment to autophagosomes via IRGM and mammalian Atg8 proteins. J Cell Biol 217, 997-1013.
Lam, S. S., Martell, J. D., Kamer, K. J., Deerinck, T. J., Ellisman, M. H., Mootha, V. K., and Ting, A. Y. (2015). Directed evolution of APEX2 for electron microscopy and proximity labeling. Nat Methods 12, 51-54.
Lazarou, M , Sliter, D. A., Kane, L. A., Sarraf, S. A., Wang, C., Burman, J. L., Sideris, D. P., Fogel, A. I., and Youle, R. J. (2015). The ubiquitin kinase PINK1 recruits autophagy receptors to induce mitophagy. Nature 524, 309-314.
Lee, J. A., Beigneux, A., Ahmad, S. T., Young, S. G., and Gao, F. B. (2007). ESCRT-III dysfunction causes autophagosome accumulation and neurodegeneration. Curr Biol 17, 1561-1567.
Levine, B., and Kroemer, G. (2019). Biological Functions of Autophagy Genes: A Disease Perspective. Cell 176, 11-42.
Lopez-Jimenez, A. T., Cardenal-Munoz, E., Leuba, F., Gerstenmaier, L., Barisch, C., Hagedom, M., King, J. S., and Soldati, T. (2018). The ESCRT and autophagy machineries cooperate to repair ESX-1 -dependent damage at the Mycobacterium-containing vacuole but have opposite impact on containing the infection. PLoS Pathog 14, e1007501.
Maejima, I., Takahashi, A., Omori, H., Kimura, T., Takabatake, Y., Saitoh, T., Yamamoto, A., Hamasaki, M., Noda, T., Isaka, Y., et al. (2013). Autophagy sequesters damaged lysosomes to control lysosomal biogenesis and kidney injury. EMBO J 32, 2336-2347.
Mandell, M. A., Jain, A., Arko-Mensah, J., Chauhan, S., Kimura, T., Dinkins, C., Silvestri, G., Munch, J., Kirchhoff, F., Simonsen, A., et al. (2014). TRIM proteins regulate autophagy and can target autophagic substrates by direct recognition. Dev Cell 30, 394-409.
Manzanillo, P. S., Shiloh, M. U., Portnoy, D. A., and Cox, J. S. (2012). Mycobacterium tuberculosis Activates the DNA-Dependent Cytosolic Surveillance Pathway within Macrophages. Cell host & microbe 11, 469-480.
Marino, G., Pietrocola, F., Eisenberg, T., Kong, Y., Malik, S. A., Andryushkova, A., Schroeder, S., Pendl, T., Harger, A., Niso-Santano, M., et al. (2014). Regulation of autophagy by cytosolic acetyl-coenzyme A. Mol Cell 53, 710-725.
Martin-Serrano, J., Zang, T., and Bieniasz, P. D. (2001). HIV-1 and Ebola virus encode small peptide motifs that recruit Tsg101 to sites of particle assembly to facilitate egress. Nat Med 7, 1313-1319.
Martina, J. A., Chen, Y., Gucck, M., and Puertollano, R. (2012). MTORC1 functions as a transcriptional regulator of autophagy by preventing nuclear transport of TFEB. Autophagy 8, 903-914.
Martina, J. A., and Puertollano, R. (2013). Rag GTPases mediate amino acid-dependent recruitment of TFEB and MITF to lysosomes. J Cell Biol 200, 475-491.
Matusek, T., Wendler, F., Poles, S., Pizerte, S., D'Angelo, G., Furthauer, M., and Therond, P. P. (2014). The ESCRT machinery regulates the secretion and long-range activity of Hedgehog. Nature 516, 99-103.
Maxfield, F. R., and McGraw, T. E. (2004). Endocytic recycling. Nat Rev Mol Cell Biol 5, 121-132.
McClelland, A., Kuhn, L. C., and Ruddle, F. H. (1984). The human transferrin receptor gene: genomic organization, and the complete primary structure of the receptor deduced from a cDNA sequence. Cell 39, 267-274.
Medina, D. L., Di Paola, S., Peluso, I., Armani, A., De Stefani, D., Venditti, R., Montefusco, S., Scotto-Rosato, A., Prezioso, C., Forrester, A., et al. (2015). Lysosomal calcium signalling regulates autophagy through calcineurin and TFEB. Nat Cell Biol 17, 288-299.
Meijer, W. H., van der Klei, I. J., Veenhuis, M., and Kiel, J. A. (2007). ATG genes involved in non-selective autophagy are conserved from yeast to man, but the selective Cvt and pexophagy pathways also require organism-specific genes. Autophagy 3, 106-116.
Mizushima, N., Noda, T., Yoshimori, T., Tanaka, Y., Ishii, T., George, M. D., Klionsky, D. J., Ohsumi, M., and Ohsumi, Y. (1998a). A protein conjugation system essential for autophagy. Nature 395, 395-398.
Mizushima, N., Sugita, H., Yoshimori, T., and Ohsumi, Y. (1998b). A new protein conjugation system in human. The counterpart of the yeast Apg12p conjugation system essential for autophagy. The Journal of biological chemistry 273, 33889-33892.
Mizushima, N., Yoshimori, T., and Ohsumi, Y. (2011). The role of Atg proteins in autophagosome formation. Annu Rev Cell Dev Biol 27, 107-132.
Morita, E., Sandrin, V., Chung, H. Y., Morham, S. G., Gygi, S. P., Rodesch, C. K., and Sundquist, W. I. (2007). Human ESCRT and ALIX proteins interact with proteins of the midbody and function in cytokinesis. EMBO J 26, 4215-4227.
Nabhan, J. F., Hu, R., Oh, R. S., Cohen, S. N., and Lu, Q. (2012). Formation and release of arrestin domain-containing protein 1-mediated microvesicles (ARMMs) at plasma membrane by recruitment of TSG101 protein. Proc Natl Acad Sci USA 109, 4146-4151.
Nabi, I. R., Shankar, J., and Dennis, J. W. (2015). The galectin lattice at a glance. J Cell Sci 128, 2213-2219.
Napolitano, G., and Ballabio, A. (2016) TFEB at a glance Journal of Cell Science In press.
Nishimura, T., Kaizuka, T., Cadwell, K., Sahani, M. H., Saitoh, T., Akira, S., Virgin, H. W., and Mizushima, N. (2013). FIP200 regulates targeting of Atg16L1 to the isolation membrane. EMBO Rep 14, 284-291.
Noda, T., and Ohsumi, Y. (1998). Tor, a phosphatidylinositol kinase homologue, controls autophagy in yeast. The Journal of biological chemistry 273, 3963-3966.
North, S. J., Huang, H. H., Sundaram, S., Jang-Lee, J., Etienne, A. T., Trollope, A., Chalabi, S., Dell, A., Stanley, P., and Haslam, S. M. (2010). Glycomics profiling of Chinese hamster ovary cell glycosylation mutants reveals N-glycans of a novel size and complexity. The Journal of biological chemistry 285, 5759-5775.
Olmos, Y., Hodgson, L., Mantell, J., Verkade, P., and Carlton, J. G. (2015). ESCRT-III controls nuclear envelope reformation. Nature 522, 236-239.
Paliikkuth, S., Martin, C., Farzam, F., Edwards, J. S., Lakin, M. R., Lidke, D. S., and Lidke, K. A. (2018). Sequential super-resolution imaging using DNA strand displacement PLoS One 13, e0203291.
Papadopoulos, C., Kirchner, P., Bug, M., Grum, D., Koerver, L., Schulze, N., Poehler, R., Dressier, A., Fengler, S., Arhzaouy, K., et al. (2017). VCP/p97 cooperates with YOD1, UBXD1 and PLAA to drive clearance of ruptured lysosomes by autophagy. EMBO J 36, 135-150.
Park, S., Buck, M. D., Desai, C., Zhang, X., Loginicheva, E., Martinez, J., Freeman, M. L., Saitoh, T., Akira. S., Guan, J. L. et al. (2016). Autophagy Genes Enhance Murine Gammaherpesvirus 68 Reactivation from Latency by Preventing Virus-Induced Systemic Inflammation. Cell Host Microbe 19, 91-101.
Patnaik, S. K., Potvin, B., Carlsson, S., Sturm, D., Leffler, H., and Stanley, P. (2006). Complex N-glycans are the major ligands for galectin-1, -3, and -8 on Chinese hamster ovary cells Glycobiology 16, 305-317.
Patnaik, S. K., and Stanley, P. (2006). Lectin-resistant CHO glycosylation mutants. Methods Enzymol 416, 159-182.
Petiot, A., Ogier-Denis, E., Blommaart, E. F., Meijer, A. J., and Codogno, P. (2000) Distinct classes of phosphatidylinositol 3′-kinases are involved in signaling pathways that control macroautophagy in HT-29 cells. The Journal of biological chemistry 275, 992-998.
Raab, M., Gentili, M., de Belly, H., Thiam, H. R., Vargas, P., Jimenez, A. J., Lautenschlaeger, F., Voituriez, R., Lennon-Dumenil, A. M., Manel, N., et al. (2016). ESCRT III repairs nuclear envelope ruptures during cell migration to limit DNA damage and cell death. Science 352, 359-362.
Radulovic, M., Schink, K. O., Wenzel, E. M., Nahse, V., Bongiovanni, A., Lafont, F., and Stenmark, H. (2018). ESCRT-mediated lysosome repair precedes lysophagy and promotes cell survival. EMBO J 37.
Randow, F., and Youle, R. J. (2014). Self and nonself: how autophagy targets mitochondria and bacteria Cell Host Microbe 15, 403-411.
Roczniak-Ferguson, A., Petit, C. S., Froehlich, F., Qian, S., Ky, J., Angarola, B., Walther, T. C., and Ferguson, S. M. (2012). The transcription factor TFEB links mTORC1 signaling to transcriptional control of lysosome homeostasis. Sci Signal 5, ra42.
Ruhl, S., Shkarina, K., Demarco, B., Heilig, R., Santos, J. C., and Broz, P. (2018). ESCRT-dependent membrane repair negatively regulates pyroptosis downstream of GSDMD activation. Science 362, 956-960.
Rusten, T. E., and Stenmark, H. (2009). How do ESCRT proteins control autophagy? J Cell Sci 122, 2179-2183.
Saxton, R. A., and Sabatini, D. M. (2017). mTOR Signaling in Growth, Metabolism, and Disease. Cell 168, 960-976.
Scheffer, L. L., Sreetama, S. C., Sharma, N., Medikayala, S., Brown, K. J., Detour, A., and Jaiswal, J. K. (2014). Mechanism of Ca(2) (+)-triggered ESCRT assembly and regulation of cell membrane repair. Nature communications 5, 5646.
Scott, R. C., Schuldiner, O., and Neufeld, T. P. (2004). Role and regulation of starvation-induced autophagy in the Drosophila fat body. Dev Cell 7, 167-178.
Searle, B. C., Pino, L. K., Egertson, J. D., Ting, Y. S., Lawrence, R. T., MacLean, B. X., Villen, J., and MacCoss, M. J. (2018). Chromatogram libraries improve peptide detection and quantification by data independent acquisition mass spectrometry. Nature communications 9, 5128.
Settembre, C., Di Malta, C., Polito, V. A., Garcia Arencibia, M., Vetrini, F., Erdin, S., Erdin, S. U., Huynh, T., Medina, D., Colella, P., et al. (2011). TFEB links autophagy to lysosomal biogenesis. Science 332, 1429-1433.
Settembre, C., Zoncu, R., Medina, D. L., Vetrini, F., Erdin, S., Huynh, T., Ferron. M., Karsenty, G., Vellard, M. C., Facchinetti, V, et al. (2012). A lysosome-to-nucleus signalling mechanism senses and regulates the lysosome via mTOR and TFEB. The EMBO journal 31, 1095-1108.
Skowyra, M. L., Schlesinger, P. H., Naismith, T. V., and Hanson, P. I. (2018). Triggered recruitment of ESCRT machinery promotes endolysosomal repair. Science 360.
Smith. C. S., Joseph, N., Rieger, B., and Lidke, K. A. (2010). Fast, single-molecule localization that achieves theoretically minimum uncertainty. Nat Methods 7, 373-375.
Stewart, S. E., Menzies, S. A., Popa, S. J., Savinykh, N., Petrunkina Harrison, A., Lehner, P. J., and Moreau, K. (2017). A genome-wide CRISPR screen reconciles the role of N-linked glycosylation in galectin-3 transport to the cell surface. J Cell Sci 130, 3234-3247.
Stolz, A., Ernst, A., and Dikic, I. (2014). Cargo recognition and trafficking in selective autophagy. Nat Cell Biol 16, 495-501.
Takahashi, Y., He, H., Tang, Z., Hattori, T., Liu, Y., Young, M. M., Serfass, J. M., Chen. L., Gebru, M., Chen, C., et al. (2018). An autophagy assay reveals the ESCRT-III component CHMP2A as a regulator of phagophore closure. Nature communications 9, 2855.
Thiele, D. L., and Lipsky, P. E. (1990). Mechanism of L-leucyl-L-leucine methyl ester-mediated killing of cytotoxic lymphocytes: dependence on a lysosomal thiol protease, dipeptidyl peptidase I, that is enriched in these cells. Proc Natl Acad Sci USA 87, 83-87.
Thurston, T. L., Wandel, M. P., von Muhlinen, N., Foeglein, A., and Randow, F. (2012). Galectin 8 targets damaged vesicles for autophagy to defend cells against bacterial invasion. Nature 482, 414-418.
Valley, C. C., Liu, S., Lidke, D. S., and Lidke, K. A. (2015). Sequential superresolution imaging of multiple targets using a single fluorophore. PLoS One 10, e0123941.
van Niel, G., Charrin, S., Simoes, S., Romao, M., Rochin, L., Saftig, P., Marks, M. S., Rubinstein, E., and Raposo, G. (2011). The tetraspanin CD63 regulates ESCRT-independent and -dependent endosomal sorting during melanogenesis. Dev Cell 21, 708-721.
Vasta, G. R., Feng, C., Gonzalez-Montalban, N., Mancini, J., Yang, L., Abernathy, K., Frost, G., and Palm, C. (2017). Functions of galectins as ‘self/non-self’-recognition and effector factors. Pathog Dis 75.
Velikkakath, A. K., Nishimura, T., Oita, E., Ishihara, N., and Mizushima, N. (2012). Mammalian Atg2 proteins are essential for autophagosome formation and important for regulation of size and distribution of lipid droplets. Molecular biology of the cell 23, 896-909.
Vietri, M., Schink, K. O., Campsteijn, C., Wegner, C. S., Schultz, S. W., Christ, L., Thoresen, S. B., Brech, A., Raiborg, C., and Stenmark, H. (2015). Spastin and ESCRT-III coordinate mitotic spindle disassembly and nuclear envelope sealing. Nature 522, 231-235.
Wang, S. F., Tsao, C. H., Lin, Y. T., Hsu, D. K., Chiang, M. L., Lo, C. H., Chien, F. C., Chen, P., Arthur Chen, Y. M., Chen, H. Y., et al. (2014). Galectin-3 promotes HIV-1 budding via association with Alix and Gag p6. Glycobiology 24, 1022-1035.
Watson, R. O., Manzanillo, P. S., and Cox, J. S. (2012). Extracellular M. tuberculosis DNA Targets Bacteria for Autophagy by Activating the Host DNA-Scnsing Pathway. Cell 150, 803-815.
Webster, B. M., Colombi, P., Jager, J., and Lusk, C. P. (2014). Surveillance of nuclear pore complex assembly by ESCRT-III/Vps4. Cell 159, 388-401.
Wei, Y., Chiang, W. C., Sumpter, R., Jr., Mishra, P., and Levine, B. (2017). Prohibitin 2 Is an Inner Mitochondrial Membrane Mitophagy Receptor. Cell 168, 224-238 e210.
Yoshida, Y., Yasuda, S., Fujita, T., Hamasaki, M., Murakami, A., Kawawaki, J., Iwai, K., Saeki, Y., Yoshimori, T., Matsuda, N., et al. (2017). Ubiquitination of exposed glycoproteins by SCF(FBXO27) directs damaged lysosomes for autophagy. Proc Natl Acad Sci USA 114, 8574-8579.
Young, A. R., Chan, E. Y., Hu, X. W., Kochl, R., Crawshaw, S. G., High, S., Hailey, D. W., Lippincott-Schwartz, J., and Tooze, S. A. (2006). Starvation and ULK1-dependent cycling of mammalian Atg9 between the TGN and endosomes. J Cell Sci 119, 3888-3900.

1. A method of treating an autophagy mediated disease in a patient in need comprising administering to said patient an effective amount of a Galectin-3 (Gal3) modulator compound , an Alix(AIP1) modulator compound, a transferrin receptor (TFRC) modulator compound or a mixture thereof.

2. The method according to claim 1 wherein method comprises administering a mixture of a Galectin-3 (Gal3) modulator compound, an Alix(AIP1) modulator compound or a transferrin receptor (TFRC) modulator compound to said patient.

3. The method according to claim 2 wherein said method comprises administering a mixture of a Galectin-3 (Gal3) modulator compound and an Alix(AIP1) modulator compound to said patient.

4. The method according to claim 2 wherein said method comprises administering a Galectin-3 (Gal3) modulator compound and a transferrin receptor (TFRC) modulator compound to said patient.

5. The method according to claim 2 wherein 1 said method comprises administering an Alix(AIP1) modulator compound and a transferrin receptor (TFRC) modulator compound to said patient.

6. The method according to claim 1 wherein said method comprises administering a Galectin-3 (Gal3) modulator compound, an Alix(AIP1) modulator compound and a transferrin receptor (TFRC) modulator compound to said patient.

7. The method according to claim 1 wherein said modulator is an autophagy agonist.

8. The method according to claim 1 wherein said modulator is an inhibitor of autophagy.

9. The method according to 6 wherein said modulator is a Gal3 agonist.

10. The method according to claim 9 wherein said Gal3 agonist is a sugar which comprises at least one galactose unit.

11. (canceled)

12. (canceled)

13. (canceled)

14. (canceled)

15. (canceled)

16. (canceled)

17. (canceled)

18. (canceled)

19. The method according to claim 1 wherein said modulator is a Alix(AIP1) agonist.

20. The method according to claim 19 wherein said modulator is a calcium salt or calcium chelate.

21. The method according to claim 20 wherein said modulator is calcium carbonate, calcium citrate, calcium gluconate, calcium lactate, calcium phosphate, dicalcium malate, calcium hydroxyapatite, coral calcium or mixtures thereof.

22. The method according to claim 1 wherein said modulator is a TFRC agonist.

23. The method according to claim 22 wherein said TFRC agonist is a ferrous salt or ferrous chelate.

24. (canceled)

25. The method according to claim 1 wherein said modulator is a Gal3 antagonist.

26. (canceled)

27. (canceled)

28. The method according to claim 1 wherein said modulator is an Alix(AIP1) antagonist.

29. The method according to claim 28 wherein said Alix(AIP1) antagonist is a calcium chelator.

30. The method according to claim 29 wherein said calcium chelator is ethylenediaminetetraacetic acid (EDTA), ethyleneglycol-bis(2-aminoethylether)N,N,N′N′tetraacetic acid (egtazic acid or EGTA), EGTA acetoxymethyl ester (EGTA AM), 1,2-Bis(2-aminophenoxy)ethane-N,N,N′,N′-tetraacetic acid (BAPTA), BAPTA acetoxymethyl ester (BAPTA AM) and Poly(vinyl phosphonic acid-co-acrylic acid) (PVPA-coAA with mole ratios of VPA to acrylic acid ranging from 20:80 to 80:20) or a pharmaceutically acceptable salt thereof.

31. The method according to claim 1 wherein said modulator is a TFRC antagonist.

32. The method according to claim 31 wherein said TFRC antagonist is an iron chelator.

33. The method according to claim 32 wherein said iron chelator is deferoxamine, deferasirox, Dp44mT, dexrazoxane, dexrazoxane HCl (ICRF-187), ciclopirox, pentetate calcium trisodium hydrate, 2,3-dihydroxybenzoic acid, VLX600, L-mimosine, N-NE3TA-NCS, CAB-NE3TA, DFT (2-(3′-hydroxypyrid-2′-yl)4-methyl-delta2-thiazoline-4(S)-carboxylic acid; desferrithiocin), 4-(OH)-DADFT, 4′-(HO)-DADMDFT ((S)-2-(2,4-dihydroxyphenyl)-4,5-dihydro-4-thiazolecarboxylic acid), BDU ((S,S)-1,11-bis[5-(4-carboxy-4,5-dihydrothiazol-2-yl)-2,4-dihydroxyphenyl]-4,8-dioxaundecane), ICL6770A (4-[3,5-bis-(hydroxyphenyl)-1,2,4-triazol-1-yl]-benzoic acid), DFP (3-Hydroxy-1,2-dimethyl-4(1H)-pyridone; Deferiprone), CP94 (Diethyl hydroxypyridinone), CP502 (1,6-dimethyl-3-hydroxy-4-(1H)-pyridinone-2-carboxy-(N-methyl)-amide hydrochloride), TREN-(Me-3,2-HOPO) (N,N′,N″-tris[(3-hydroxy-1-methyl-2-oxo-1,2-didehydropyrid-4-yl)carboxamidoethyl]amine), Pr-(Me-3,2-HOPO) (3-Hydroxy-1-methyl-2-oxo-1,2-dihydro-pyridine-4-carboxylic acid propylamide), Tachpyridine (N,N′,N″-tris(2-pyridylmethyl)-cis,cis-1,3,5-triaminocyclohexane),PIH, SIH (Salicylaldehyde isonicotinoyl hydrazone), 311 (2-hydroxy-1-naphthylaldehyde isonicotinoyl hydrazone), 5-HP (5-hydroxypicolinaldehyde thiosemicarbazone), D-Exo 772SM, PCIH, INH, Deferitazole, EDTA, DTPA, Succimer, Trientine, BPS, PCTH, PCBH, PCBBH, PCAH, PCHH, FIH, Quercetin, or a pharmaceutically acceptable salt or mixture thereof.

34. The method according to wherein said autophagy mediated disease state is a microbial infection, an inflammatory disorder, a lysosomal storage disorder, an immune disorder, cancer or a neurodegenerative disorder.

35. (canceled)

36. (canceled)

37. (canceled)

38. (canceled)

39. (canceled)

40. The method according to claim 1 wherein said autophagy mediated disease state is Alzheimer's disease, Parkinson's disease, Huntington's disease; inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease; hyperglycemic disorders, diabetes (I and II), severe insulin resistance, hyperinsulinemia, insulin-resistant diabetes, dyslipidemia, depressed high-density lipoprotein (HDL), and elevated triglycerides, liver disease, renal disease, cardiovascular disease, including infarction, ischemia, stroke, pressure overload and complications during reperfusion, muscle degeneration and atrophy, symptoms of aging, low grade inflammation, gout, silicosis, atherosclerosis, age-associated dementia and sporadic form of Alzheimer's disease, psychiatric conditions including anxiety and depression, spinal cord injury, arteriosclerosis or a bacterial, fungal, cellular or viral infections.

41. (canceled)

42. (canceled)

43. A pharmaceutical composition for use in treating an autophagy mediated disease in a patient in need comprising an effective amount of a mixture of a Galectin-3 (Gal3) modulator compound, an Alix(AIP1) modulator compound and/or a transferrin receptor (TFRC) modulator compound in combination with a pharmaceutically acceptable carrier, additive or excipient.

44. The composition according claim 43 comprising a mixture of a Galectin-3 (Gal3) modulator compound, an Alix(AIP1) modulator compound and a transferrin receptor (TFRC) modulator compound.

45-74. (canceled)
